(19) United States
(12) Patent Application Publication
(10) Pub. No.: US 2017/0146391 A1
(43) Pub. Date: May 25, 2017

(54) SCALE WITH FOOT-CONTROLLED USER INTERFACE

(71) Applicant: Physiowave, Inc., Santa Clara, CA (US)

(72) Inventors: Gregory T. Kovacs, Palo Alto, CA (US); Richard M. Wiard, Campbell, CA (US)

(21) Appl. No.: 15/354,985

(22) Filed: Nov. 17, 2016

Related U.S. Application Data
(63) Continuation of application No. PCT/US2016/062484, filed on Nov. 17, 2016.
Application No. PCT/US2016/062484 is a continuation of application No. PCT/US2016/062505, filed on Nov. 17, 2016.
(60) Provisional application No. 62/258,238, filed on Nov. 20, 2015.
(60) Provisional application No. 62/265,841, filed on Dec. 10, 2015.
(60) Provisional application No. 62/266,523, filed on Dec. 11, 2015.

(51) Int. Cl. G01G 23/18; G01G 23/00; G06F 3/0488; G06F 3/0481; G06F 3/0484; A61B 5/00; A61B 5/0205; A61B 5/04; A61B 5/0408; A61B 5/0432; A61B 5/044; A61B 5/1174; A61B 5/0478; A61B 5/11; G01G 19/50

(52) U.S. Cl. CPC G01G 23/18; G01G 19/50; G01G 23/007; G06F 3/04886; G06F 3/04883; G06F 3/04817; G06F 3/04842; A61B 5/0022; A61B 5/02055; A61B 5/04012; A61B 5/04085; A61B 5/0432; A61B 5/044; A61B 5/1174; A61B 5/486; A61B 5/6887; A61B 5/7221; A61B 5/7465; A61B 5/7495; A61B 5/0478; A61B 5/1102; A61B 5/1128; A61B 5/0075; A61B 2090/064

(57) ABSTRACT

Certain aspects of the disclosure are directed to a weighing scale including a platform, data-procurement circuitry, processing circuitry and foot-controlled user interface (FUI). The processing circuitry is electrically integrated with the data-procurement circuitry to process data obtained by the data-procurement circuitry while the user is standing on the platform and therefrom generate cardio-related physiologic data. The FUI provides data to the user while the user is standing on the platform and receive a foot-based user input from the user for the user to interact with the weighing scale.

### RELATED APPLICATION DATA

This application is related to PCT Application (Ser. No. PCT/US2016/062505), entitled “Remote Physiologic Parameter Assessment Methods and Platform Apparatuses”, filed on Nov. 17, 2016, PCT Application (Ser. No. PCT/US2016/062484), entitled “Scale-Based Parameter Acquisition Methods and Apparatuses”, filed on Nov. 17, 2016, U.S. Provisional Application (Ser. No. 62/258,238), entitled “Condition or Treatment Assessment Methods and Platform Apparatuses”, filed Nov. 20, 2015, U.S. Provisional Application (Ser. No. 62/265,841), entitled “Scale With Foot-Controlled User Interface”, filed Dec. 10, 2015, and U.S. Provisional Application (Ser. No. 62/266,523), entitled “Social Grouping Using a User-Specific Scale-Based Enterprise System”, filed Dec. 11, 2015″, which are fully incorporated herein by reference.

### SUMMARY OF THE DISCLOSURE

Various aspects of the present disclosure are directed toward methods, systems and apparatuses that include a foot-controlled user interface (FUI).

A variety of aspects are directed to monitoring different physiological characteristics many different applications. For instance, physiological monitoring instruments are often used to measure a number of patient vital signs, including blood oxygen level, body temperature, respiration rate and electrical activity for electrocardiogram (ECG) or electroencephalogram (EEG) measurements. For ECG measurements, a number of electrocardiograph leads may be connected to a patient's skin, and are used to obtain a signal from the patient.

Obtaining physiological signals (e.g., data) can often require specialty equipment and intervention with medical professionals. For many applications, such requirements may be costly or burdensome. These and other matters have presented challenges to monitoring physiological characteristics.

Aspects of the present disclosure are directed to a platform apparatus that provides various features using a FUI located at a platform of the platform apparatus, such as a body weight scale. The FUI is a user interface that allows the user to interact with the platform apparatus using user inputs to the FUI. Such user inputs include movement of the user's foot relative to the FUI, contacting the FUI with a foot of the FUI, tapping the FUI with a foot, the user shifting their weight between feet and/or front to back (e.g., toe to heel or vice versa), among other inputs. In various specific aspects, the FUI is used to provide (e.g., display) alerts correlated to scale-obtained data of the users. Such alerts includes notifications of social groupings available for the user that are tailored to the scale-obtained data, additional information regarding social groupings and/or health information, and/or an indication that additional information is available for the user on an standalone CPU in communication with the platform apparatus, such as a smartphone, tablet and/or computing device. In specific aspects, the additional information includes various advertisements, such as products and services available and correlated with the scale-obtained data, generic health information, updates on the social groupings, available diagnosis information from a physician, requests for participation in studies by a physician and/or other research, among other data. The platform apparatus, using the FUI, additionally provides such features as discerning an amount and location of data to display between the FUI and a graphical user interface of another user device based on user demographic information, the data to display, and/or prior user-specific use of the platform apparatus. In various aspects, the platform apparatus is configured to operate in different communication modes using the FUI, registers different biometrics, and/or displays data using color coding that is indicative of data measured and used to abbreviate more complex information.

A user interface includes or refers to interactive components of a device (e.g., the scale) and circuitry configured to allow interaction of a user with the scale (e.g., hardware input/output components, such as a screen, speaker components, keyboard, touchscreen, etc., and circuitry to process the inputs). A user display includes an output surface (e.g., screen) that shows text and/or graphical images as an output from a device to a user (e.g., cathode ray tube, liquid crystal display, light-emitting diode, organic light-emitting diode, gas plasma, touch screens, etc.)

Certain aspects of the present disclosure is directed to apparatuses and methods including a scale with a FUI. The scale includes a platform for a user to stand on, data-procurement circuitry, processing circuitry, a user display, and output circuitry. The data-procurement circuitry includes force sensor circuitry and a plurality of electrodes integrated with the platform for engaging the user with electrical signals and collecting signals indicative of the user's identity and cardio-physiological measurements while the user is standing on the platform. The processing circuitry includes a CPU and a memory circuit with user-corresponding data stored in the memory circuit. The processing circuitry is arranged with (e.g., electrically integrated with or otherwise in communication) data obtained by the data-procurement circuitry while the user is standing on the platform and therefrom generate cardio-related physiologic data corresponding to the collected signals. The user display is configured for displaying a FUI including data through at least a portion of the platform whereon the user stands. The output circuit receives and outputs data from the processing circuitry to the user display for viewing by the user through the platform.

In various specific aspects, the user display includes a touch screen and/or is associated with motion sense circuitry and/or accelerometers. The FUI enables the user to interact with the scale apparatus. For example, the user interaction is by the user's foot with the user display and causes the FUI to undergo a change in appearance.

Various specific aspects include methods for providing (e.g., displaying) data using a FUI. For example, various method embodiments transitioning a scale, in response to a user approaching or standing on a platform of the scale, from a reduced power-consumption mode of operation to at least one higher power-consumption mode of operation. The higher power-consumption mode, for example, includes activating a FUI on a user display of the scale. The scale includes a platform for a user to stand on, data-procurement circuitry, processing circuitry, a user display, and an output circuit. The data-procurement circuitry includes force sensor circuitry and a plurality of electrodes integrated with the platform. The processing circuitry includes a CPU and a memory circuit with user-corresponding data stored in the memory circuit. The processing circuitry is arranged within the scale and under the platform upon which the user stands and is electrically integrated with the force sensor circuitry and the plurality of electrodes.

The method further includes engaging the user with electrical signals, using the data-procurement circuitry, and collecting signals indicative of the user's identity and cardio-physiological measurements while the user is standing on the platform. Data obtained by the data-procurement circuitry is processed, using the processing circuitry, while the user is standing on the platform and cardio-related physiologic data corresponding to the collected signals is generated therefrom. Further, user inputs are received, using the FUI, from the user's foot. The FUI allows the user to interact with the scale. For example, the user's weight is displayed using the FUI on the user display.

In certain embodiments, aspects are implemented in accordance with and/or in combination with aspects of the underlying PCT Application (Ser. No. PCT/US2016/062505), entitled “Remote Physiologic Parameter Assessment Methods and Platform Apparatuses”, filed on Nov. 17, 2016, PCT Application (Ser. No. PCT/US2016/062484), entitled “Scale-Based Parameter Acquisition Methods and Apparatuses”, U.S. Provisional Application (Ser. No. 62/258,238), entitled “Condition or Treatment Assessment Methods and Platform Apparatuses”, filed Nov. 20, 2015, U.S. Provisional Application (Ser. No. 62/265,841), entitled “Scale With Foot-Controlled User Interface”, filed Dec. 10, 2015, and U.S. Provisional Application (Ser. No. 62/266,523), entitled “Social Grouping Using a User-Specific Scale-Based Enterprise System”, filed Dec. 11, 2015″, which are fully incorporated herein by reference.

The above discussion/summary is not intended to describe each embodiment or every implementation of the present disclosure. The figures and detailed description that follow also exemplify various embodiments.

### BRIEF DESCRIPTION OF THE DRAWINGS

Various example embodiments may be more completely understood in consideration of the following detailed description in connection with the accompanying drawings, in which:

FIG. 1A shows an example of an apparatus with a FUI consistent with various aspects of the present disclosure;

FIG. 1B shows an example process for controlling display of data using a FUI consistent with various aspects of the present disclosure;

FIG. 1C shows an isometric view of a multifunction scale with large-area display, consistent with various aspects of the present disclosure;

FIG. 1D shows an isometric, cross-sectional view of a multifunction scale with large-area display, consistent with various aspects of the present disclosure;

FIG. 2 shows a top view of a multifunction scale with large-area display, consistent with various aspects of the present disclosure;

FIGS. 3A-D show top views of a number of multifunction scale displays, consistent with various aspects of the present disclosure;

FIG. 3E shows an example of a FUI, consistent with various aspects of the present disclosure;

FIG. 4 shows a multifunction scale with large-area display, consistent with various aspects of the present disclosure;

FIG. 5A is a flowchart illustrating an example manner in which a user-specific physiologic meter/scale may be programmed to provide features consistent with aspects of the present disclosure;

FIG. 5B shows current paths through the body for the IPG trigger pulse and Foot IPG, consistent with various aspects of the present disclosure;

FIG. 6A shows an example of the insensitivity to foot placement on scale electrodes with multiple excitation and sensing current paths, consistent with various aspects of the present disclosure;

FIGS. 6B-6C show examples of electrode configurations, consistent with various aspects of the disclosure;

FIG. 7A depicts an example block diagram of circuitry for operating core circuits and modules, including, for example, those of FIGS. 8A-8B, used in various specific embodiments of the present disclosure;

FIG. 7B shows an exemplary block diagram depicting the circuitry for interpreting signals received from electrodes;

FIGS. 8A-8B show example block diagrams depicting the circuitry for sensing and measuring the cardiovascular time-varying IPG raw signals and steps to obtain a filtered IPG waveform, consistent with various aspects of the present disclosure;

FIG. 9 shows an example block diagram depicting signal processing steps to obtain fiducial references from the individual Leg IPG “beats,” which are subsequently used to obtain fiducials in the Foot IPG, consistent with various aspects of the present disclosure;

FIG. 10 shows an example flowchart depicting signal processing to segment individual Foot IPG “beats” to produce an averaged IPG waveform of improved SNR (signal-to-noise ratio), which is subsequently used to determine the fiducial of the averaged Foot IPG, consistent with various aspects of the present disclosure;

FIG. 11 shows an example configuration for obtaining the pulse transit time (PTT), using the first IPG as the triggering pulse for the Foot IPG and ballistocardiogram (BCG), consistent with various aspects of the present disclosure;

FIG. 12 shows another example of a scale with interleaved foot electrodes to inject and sense current from one foot to another foot, and within one foot, consistent with various aspects of the present disclosure;

FIG. 13A shows another example of a scale with interleaved foot electrodes to inject and sense current from one foot to another foot, and to measure Foot IPG signals in both feet, consistent with various aspects of the present disclosure;

FIG. 13B shows another example of a scale with interleaved foot electrodes to inject and sense current from one foot to another foot, and to measure Foot IPG signals in both feet, consistent with various aspects of the present disclosure;

FIG. 13C shows another example approach to floating current sources by using transformer-coupled current sources, consistent with various aspects of the present disclosure;

FIGS. 14A-D show an example breakdown of a scale with interleaved foot electrodes to inject and sense current from one foot to another foot, and within one foot, consistent with various aspects of the present disclosure;

FIG. 15 shows an example block diagram of circuit-based building blocks, consistent with various aspects of the present disclosure;

FIG. 16 shows an example flow diagram, consistent with various aspects of the present disclosure;

FIG. 17 shows an example scale communicatively coupled to a wireless device, consistent with various aspects of the present disclosure; and

FIGS. 18A-C show example impedance as measured through different parts of the foot based on the foot position, consistent with various aspects of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DESCRIPTION

Aspects of the present disclosure are believed to be applicable to a variety of different types of apparatuses, systems, and methods involving scale with a foot-controlled user interface (FUI). In certain implementations, aspects of the present disclosure have been shown to be beneficial when used in the context of a weighing scale with electrodes configured for engaging with the user and generating cardio-related physiologic data, such as data indicative of a BCG or ECG of a user, and providing (e.g., displaying) portions of the data using the FUI. In some embodiments, the user interacts with the scale and causes the FUI to undergo a change in appearance. The user interaction includes the user moving their foot, the user contacting a specific portion of the FUI with their foot, the user shifting weight, among various other foot-based interactions. These and other aspects can be implemented to address challenged, including those discussed in the background above. While not necessarily so limited, various aspects may be appreciated through a discussion of examples using such exemplary contexts.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

Various embodiments are directed to a platform apparatus that provide various features using a FUI located at a platform of the platform apparatus. The platform apparatus, such as a body weight scale, can provide (e.g., displays) the FUI using a user display. In other embodiments, the FUI is provided using computer-generated voice messages via a speaker component of the scale. The FUI allows the user to interact with the platform apparatus using foot-based user inputs and/or interactions with the FUI. In various specific aspects, the FUI is used to provide (e.g., display and/or via sound) alerts correlated to scale-obtained data of the users. Such alerts includes notifications of social groupings available for the user that is tailored to the scale-obtained data, additional information regarding social groupings and/or health information, and/or an indication that additional information is available for the user on an standalone central processing unit (CPU) in communication with the platform apparatus, such as a smartphone, tablet and/or computing device. The additional information includes various advertisements, such as products and services available and correlated with the scale-obtained data, generic health information, updates on the social groupings, available diagnosis information from a physician, requests for participation in studies by a physician and/or other research, among other data.

The platform apparatus, using the FUI, additionally provides such features as discerning an amount and location of data to provide (e.g., display) between the FUI and a graphical user interface of another device based on user demographic information, the data to display, and/or prior user use of the platform apparatus. In various aspects, the user can configure the scale to operate in different communication modes using the FUI, registers different biometrics, and/or the FUI displays data using color coding that is indicative of data measured and used to abbreviate more complex information.

For ease of references, the following disclosure refers to the FUI as displaying data and using a user display. However, embodiments in accordance with the present disclosure are not so limited. For example, the FUI can provide data via computer generated voice messages, haptic responses, and/or other sounds. In other embodiments, the scale includes a user interface other than or in addition to a FUI. In various embodiments, the scale includes one or more speaker components to provide the data to the user. Thereby, the FUI can include a projection of sound via the speaker components. A user interface includes or refers to interactive components of a device (e.g., the scale) and circuitry configured to allow interaction of a user with the scale (e.g., hardware input/output components, such as a screen, speaker components, keyboard, touchscreen, etc., and circuitry to process the inputs). A user display includes an output surface (e.g., screen) that shows text and/or graphical images as an output from a device to a user (e.g., cathode ray tube, liquid crystal display, light-emitting diode, gas plasma, touch screens, etc.) In various embodiments, the user interface includes a FUI. A FUI allows for the user to interact with the scale via inputs using their foot. A FUI includes or refers to a user interface that receives inputs from the user's foot (e.g., via the platform) to allow the user to interact with the scale. The user interaction includes the user moving their foot relative to the FUI, the user contacting a specific portion of the user display, the user shifting their weight, etc. Example GUIs include input/output devices, such as display screens, touch screens, microphones, etc.

In specific embodiments, the FUI can be used to instruct the user to have particular posture while taking measurements and until an alert is provided to the user. The instruction, in various embodiments, directs the user to remain still and/or look forward (e.g., “hold your head up and look forward”). In response to the instruction, the scale collects physiologic data from the user. And, in response to the collection of physiological data and/or verification that the user has a specific/current posture, the processing circuitry, and the user display and/or a speaker, provide an alert to the user. The alert indicates that the user may lower their head, such as indicating that the user can look at the user display of the apparatus, and/or the user may move.

Various aspects of the present disclosure are directed toward a multifunction scale with a user display to present results of the scale's multiple sensing functionalities, as well as other information pertinent to a user. In many embodiments, the scale is capable of a number of biometric and physiological measurements. Based on the measurements, a condition(s) of the user is displayed on the user display between or beneath the user's feet. As the user display is located near the user's feet, it may be difficult to interact with the scale during various processing of the biometric and physiological measurements. Embodiments in accordance with the present disclosure include a FUI that enables the user to interact with the scale via user interaction by the user's foot. The user interaction causes the FUI to undergo a change in appearance and/or cause the scale to perform a function, as discussed further herein.

In accordance with a number of embodiments, physiological parameter data is collected using an apparatus, such as a weighing scale or other platform that the user stands on. The user (owners of a scale or persons related to the owner, such as co-workers, friends, roommates, colleagues), may use the apparatus in the home, office, doctors office, or other such venue on a regular and frequent basis, the present disclosure is directed to a substantially-enclosed apparatus, as would be a weighing scale, wherein the apparatus includes a platform which is part of a housing or enclosure and a user display to output user-specific information for the user while the user is standing on the platform. The platform includes a surface area with electrodes that are integrated and configured and arranged for engaging a user as he or she steps onto the platform. Within the housing is processing circuitry that includes a CPU (e.g., one or more computer processor circuits) and a memory circuit with user-corresponding data stored in the memory circuit. The platform, over which the electrodes are integrated, is integrated and communicatively connected with the processing circuitry. The processing circuitry is programmed with modules as a set of integrated circuitry which is configured and arranged for automatically obtaining a plurality of measurement signals (e.g., signals indicative of cardio-physiological measurements) from the plurality of electrodes. The processing circuitry generates, from the signals, cardio-related physiologic data manifested as user-data.

In various embodiments, the user display of the scale displays the FUI including data through the platform. A FUI is a user interface that allows for the user to interact with the scale via inputs using their foot and/or via graphic icons or visual indicators near the user's foot while standing on the platform. For example, the data is displayed through at least a portion of the platform whereon the user stands. The user interacts with the scale by foot-based user interaction. For example, the FUI receives inputs from the user's foot to allow the user to interact with the scale. The user interaction includes the user moving their foot relative to the FUI, the user contacting a specific portion of the user display, the user shifting their weight, etc. In some specific embodiments, the user display includes a touch screen and the user interaction includes the user selecting an icon, an item in a list, a virtual keyboard, among other selections, using a portion of their foot.

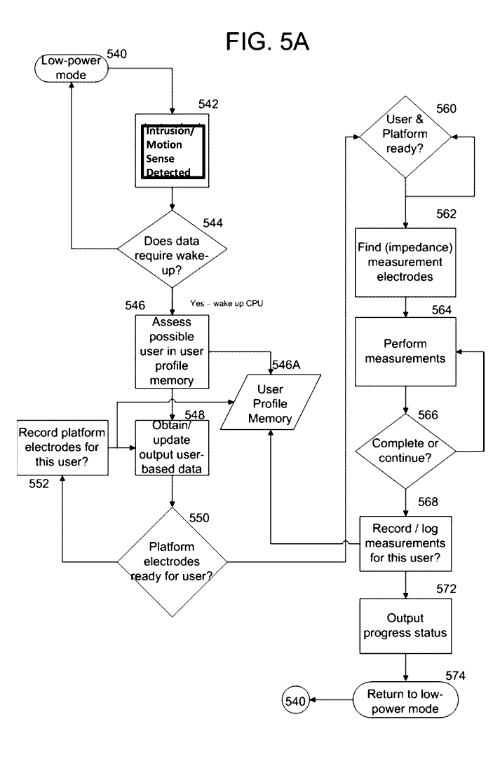

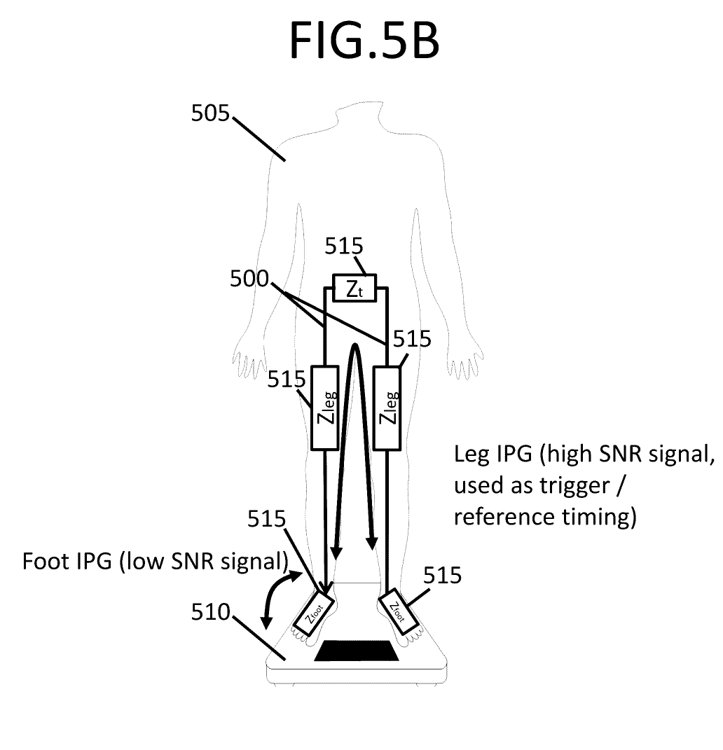

In various specific embodiments, the data procurement circuitry collects physiological data from the user such as measurements of body composition and cardiovascular information which are then forwarded on to the processing circuitry for analysis. The user display displays data through and throughout the entire platform, including entertainment information and physiological parameters of the user as determined by the data-procurement circuitry and processing circuitry. The processing circuitry receives the cardio-related measurements from the sensor circuitry and generates cardio-related physiologic data of the user, including a user-weight, while the user stands on and engages the sensor circuitry of the platform. Methodologies for determining physiological parameters of the user are discussed in more detail below, in reference to FIGS. 5-18C. The output circuitry receives the information from the processing circuitry and provides the information to the display for viewing by the user through the platform.

In some embodiments exemplified by the present disclosure, to determine the identity of a user (without selection by the user, or some other method of identification), the memory circuit of the processing circuitry includes user-corresponding data stored thereon. The user-corresponding data includes an indication of a scale-based biometric that is used to identify the user. For example, the processing circuitry can utilize characteristics of a user's cardiogram (e.g., QRS complex), toe print, the user's resting heart rate, approximate weight, etc. (or a combination thereof) to determine the identity of the user. In yet further embodiments, based on the identification of the user by the processing circuitry, the processing circuitry may access information, via a communication network, based on the user-specific data in the memory circuit (e.g., sports scores for teams the user likes, weather in the user's area, stock prices associated with the user's financial portfolio, etc.). Furthermore, the user-specific data can include various user preferences (e.g., personal settings) for the FUI, such as display settings (text size, color schemes), functions frequently performed, what data to display, and other user preferences.

Biometrics, as used herein, are metrics related to human characteristics and used as a form of identification and access control. Scale-based biometrics includes biometrics that are obtained using signals collected by the data-procurement circuitry of the scale (e.g., using electrodes and/or force sensors). Example scale-based biometrics include foot length, foot width, weight, voice recognition, facial recognition, a passcode tapped and/or picture drawn with a foot of the user on the FUI/GUI of the user display, among other biometrics. In some specific embodiments, a scale-based biometric includes a toe-print (e.g., similar to a finger print) that is recognized using a toe-print reader on the FUI/GUI of the scale. The toe print can be used as a secure identification of the user. In other embodiments, the scale-based biometric includes a finger print captured using external circuitry in communication with the scale (e.g., a cellphone or tablet having finger print recognition technology). For example, a wearable device, such as a ring, wristband, and/or ankle bracelet can be used to positively identify a user, with or without biometrics.

In many embodiments of the present disclosure, the display functions as a touch-screen, wherein the user display receives user input (e.g., touch signal data) indicative of engagement of the user on the platform and the associated position and movement of the user's feet. The output circuit receives the user input from the user display, processes the touch signals, and determines the position and movement associated with such touch signals. For example, the FUI can display various tests and/or functions that can be performed and the user can select one of the test or functions by contacting their toe with an icon of the respective test or function. In response to the selection, the scale performs the test or function. The user's feet may be used to make a number of selections as to the functionality of the scale including what physiological tests to conduct, what information to display, etc. Alternatively and/or in addition, the scale is configured with a haptic, capacitive or flexible pressure-sensing upper surface, the (upper surface/tapping) touching from or by the user is sensed in the region of the surface and processed according to conventional X-Y grid Signal processing in the logic circuitry/CPU that is within the scale. By using one or more of the accelerometers located within the scale at its corners, such user data entry is sensed by each such accelerometer so long as the user's toe, heel or foot pressure associated with each tap provides sufficient force. In some embodiments, the user display is integrated with motion sense circuitry. The user interaction, in such embodiments, include the user moving their foot (with or without touching the user display).

In specific embodiments, the control of the FUI can be provided to a separate user device, such a user device that has previously been or is paired with the scale and that is detected by the scale. As a specific example, the scale provides a cellphone with control functions to control the display of the FUI in response to detecting the cellphone is within a threshold distance.

The user display is configured and arranged for providing data through at least a portion of platform whereon the user stands. In some embodiments, the output circuit is further configured and arranged to present information including images via the FUI when the user is not standing on the platform. For example, the output circuit provides information from the processing circuitry to present the information through the platform for viewing by the user in a first display mode corresponding to a first state in which the user is not standing on the platform and for viewing by the user in a second display mode corresponding to a second state in which the user is standing on the platform. The different modes can, for example, include different font or image size, different placement of the FUI (e.g., to take into account where the user is standing), and different items displayed. This is advantageous as it permits for the images, icons, and/or text to be displayed in portions of the FUI that the user is not blocking with their feet. For example, when a user is not standing on the platform, a larger portion of the user display may be used to display data. Accordingly, the areas which display information and/or the types of information (e.g., images, text, user-specific alpha-numeric, and generic data such as weather, time of day, etc.) can be controlled in response to determining whether the user is standing on the scale, approaching the scale, getting off the scale, and thereby transitioning the displayed information and view as a function of the user's access/proximity to the scale.

In various embodiments a multifunction scale including a display is disclosed, the display being effectively the entire top surface of the scale. Support glass above the display transmits the weight of a user to a bezel along the perimeter of the scale (away from the display), while also transmitting touch-capacitive signals indicative of a user's position and movement on the support glass through the display to scale circuitry. The bezel houses load cells equally spaced along the perimeter of the scale. Each load cell outputs an electrical signal indicative of a mass transmitted from the user through the load cell to the scale circuitry. A support frame is attached to the bezel and supports the display within the bezel. A plurality of translucent electrode leads are embedded into the support glass to provide electrical signals to the scale circuitry; the electrical signals are interpreted by the scale circuitry as being indicative of a condition of a user, such a condition being presented on the display for the user.

In some embodiments of the present disclosure, a display of a multifunction scale is touch-responsive or tilt-responsive. The FUI may portray simple menus that can be controlled by the user's feet/toes. A user's feet are sensed via touch sensors on the screen or display and the scale can identify the outline of a user's feet (or hands or other body part). The user's feet may provide user input for functional or aesthetic feedback via the user display such as producing animated graphics around the user's feet (e.g., simulated lapping surf videos that interact with the user's feet; glowing around the user's feet; fish nibbling at the toes, etc.). In other specific embodiments, the graphics and/or text convey videos, simulations of motion of solids or liquids, of animals and/or water encompassing the user's feet, thereby helping to relax or relate the user to the tools with which the scale is equipped.

In yet other specific embodiments, the graphics and/or text are interactive so that while the user stands on the platform, the displays shows information for the user's foot-limited field of view, thereby permitting the user to view relevant portions of graphics and/or text for discernible communication from the display to the user (e.g., displaying information to encourage the user eat healthier, slow-down the rate of breathing, exercise more, relax with a relaxing display such as moving clouds and/or simply relay other information such as weather, news and traffic data). For example, specific information (e.g., that is important) can display in areas of the display that are not covered by the user's feet. A user may also change posture, shifting the weight distribution over the scale's load cells to provide user input. In specific embodiments, the graphics or text are displayed prior to the user standing on the scale using the entire user display and when the user stands on the platform, the graphics or text move to locations of the user display that are not covered by the user's feet. The movement of the graphics or text can be similar to liquid flowing (e.g., flowing from the first location that is centered on the user display to locations that are not covered by the user's feet).

In certain other embodiments, the graphics and/or text are interactive so that while the user stands on the platform, the displays shows information/advertisements that are relevant to user data stored in the scale's internal (or externally coupled as in the case of a handheld device) memory circuit. The interactivity of the user, e.g., tapping the scale with the user's foot as an input, can be tracked and relayed for further correlation in the memory circuit and to provide other related information in response. As non-limiting examples, this (other) information can be responsive to the user's weight or indications of heart-related parameters (such as cholesterol and/or arterial stiffness) via 360-degree interactivity from the user as measured on the scale, to displayed user data while the user is standing on the scale, and to the memory circuit and/or an Internet server whereby correlation and tracking to user-related information can be tracked and scored. Information from such an Internet server (as operated by a third party) can also be accessed and displayed as part of a medical/fitness—related suggestion. Examples include displaying information to the effect of: exercise moderately each day for a week and return to scale periodically for a report on your progress; and as it appears that you have symptoms consistent with cholesterol and/or arterial stiffness, ask your doctor if you should be taking medication known as [medication name]. Such information, in some embodiments, is displayed responsive to the user stepping off the scale and displayed in larger font and/or using a larger portion of the user display so that user may view the text while standing.

The user provided feedback allows for the selection of menu options, test selection, browsing information or articles presented on the display, or the input of test relevant user data such as age, medical conditions, etc. In various embodiments, the FUI indicates to scale circuitry the location of a user's feet relative to a plurality of electrodes located across a top surface of the multifunction scale.

In further, more specific, embodiments of the present disclosure, a multifunction scale is communicatively coupled with a user's portable electronic devices, an internet router, or other home electronic devices. The scale then communicates and exchanges data with these devices for display and control by a user (e.g. using physiological parameters to improve a fitness or health condition). In various embodiments, the FUI displays data using a color coding to abbreviate more complex information, such as scale-obtained data. As an example, a green color indicates a good value, a yellow color indicates an okay value, and a red color indicates a bad value. Such values include physiologic parameters, weight, weight increase or decrease, recovery parameters, among other information. The scale communicates the information with other electronic devices, including the color coding, such that the more complex information is displayed on the other electronic device using the same color coding. The electronic communications between the multifunction scale and the various devices may take the form of either wireless or wired communications. Further, a multifunction large display scale can be used to communicate with other scale users either using the same scale unit or another scale in the home or other wireless or personal electronic devices (e.g. leaving someone a message or note or confirming a meeting or appointment; and/or incorporating the digital communication and haptic feedback system from a smart watch to make selections related to scale functionality).

Aspects of the present disclosure are directed toward a multifunction scale that obtains a plurality of impedance-measurement signals while a set of at least three electrodes are concurrently contacting a user. Additionally, various aspects of the present disclosure include determining a plurality of pulse characteristic signals based on the plurality of impedance-measurement signals. One of the pulse characteristic signals is extracted from one of the impedance-measurement signals and is used as a timing reference to extract and process another of the pulse characteristic signals. The signals obtained by the scale are indicative of a condition of the user, such as percentage: muscle mass percentage, body water percentage, among others. The condition of the user is displayed on a large-area display beneath the user's feet, along with other information that may be preprogrammed or requested by the user for display such as time of day, traffic conditions, stock portfolio, weather, as well as a plurality of other pieces of information that may be collected.

In a further embodiment of the present disclosure, a multifunction scale includes circuitry such as a camera, microphone or image processing circuitry that interacts with an external environmental sensor. Such an environmental sensor may, for example, be connected to a personal electronic device to alert a user of motions and sounds in a house, or to communicate wirelessly with another individual either nearby or at a distant location. In some implementations, the scale communicates with and relies on an external environmental sensor that is wirelessly connected to the user's home or living environment. For example, in one embodiment the external environmental sensors facilitate power saving by alerting the scale that a user is moving toward the location of the scale, thereby prompting the scale to transition (turn on or power up) from idle or reduced-display mode to active or large-display mode, identify the user, and begin interacting with the user. Further, the external environmental sensor can also trigger the scale to turn off or to transition from active mode or large-display mode to a reduced-display or idle mode, in response to sensing that the user is leaving the area where the scale is located.

In one power-saving embodiment of the present disclosure, the scale display is operated in a first (large-area) display mode, where the entirety of the surface of the scale platform consists of the display when the user is not standing on the scale; and a second (smaller or reduced-area) display mode, when the user is standing on the scale (e.g., the portions of the display visible to the user). In some embodiments, only a small portion of the display between the user's feet will continue to display information to the user in the second display mode, and in other embodiments the second display mode turns-off the display area under the user's feet to save battery power.

In another power saving embodiment of the disclosure, the scale may operate in an active mode, determined by the presence of the user by a camera or microphone integrated onto the scale. When image processing circuitry associated with the camera senses motion (or the microphone circuitry detects a noise), the scale enters an active mode and presents information that corresponds to the physiological parameters of the user or other information as may be programmed by the user. In the alternative, idle mode, where the scale has been inactive for a programmed period of time, or the image processing circuitry and microphone circuitry determine the lack of user presence in the room, the scale may turn-off the display to save power, present an image indicative of the area around the base unit, or present and image or animation selected by the user (as may be desired by the user).

The above discussion/summary is not intended to describe each embodiment or every implementation of the present disclosure. The figures and detailed description that follow also exemplify various embodiments.

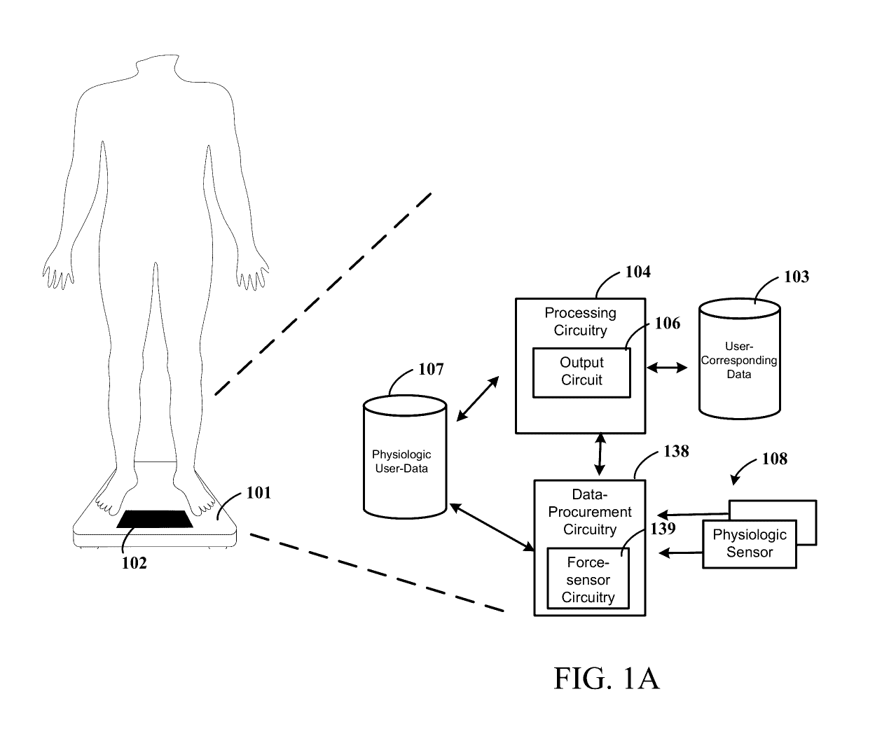

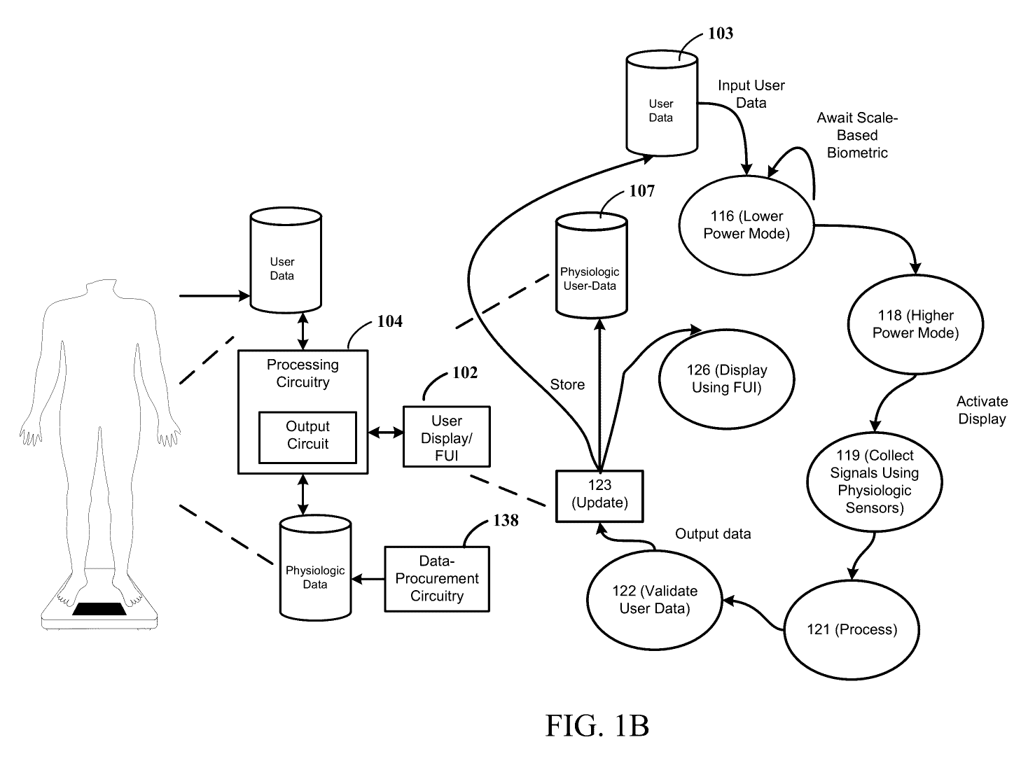

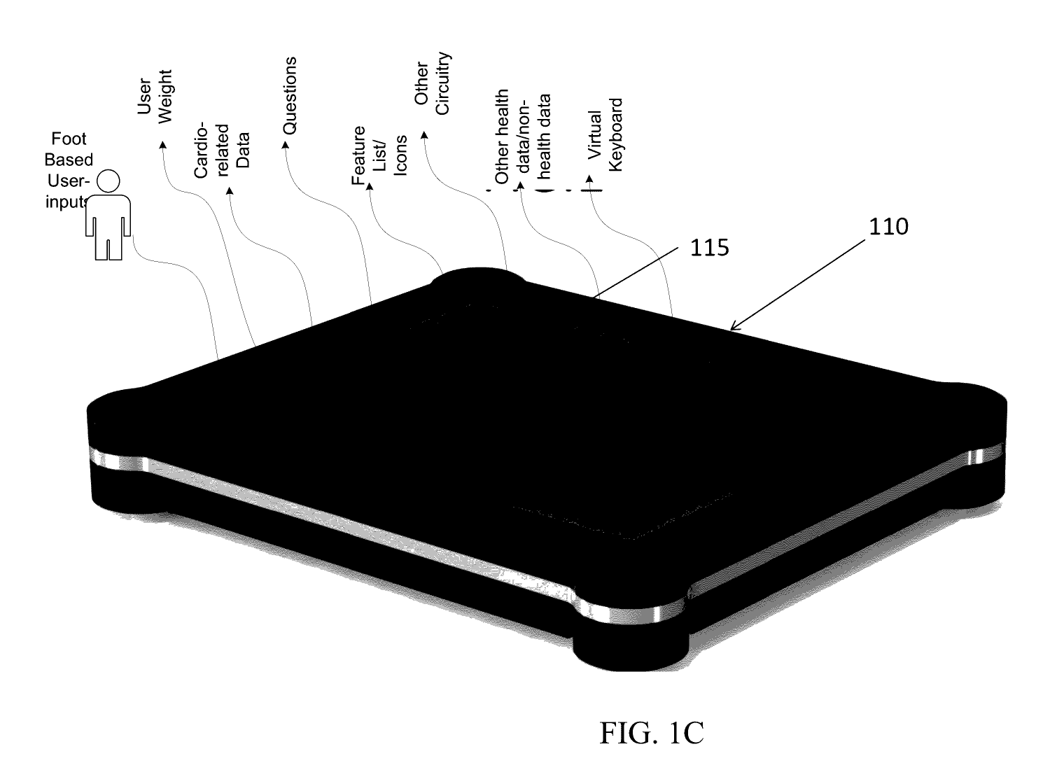

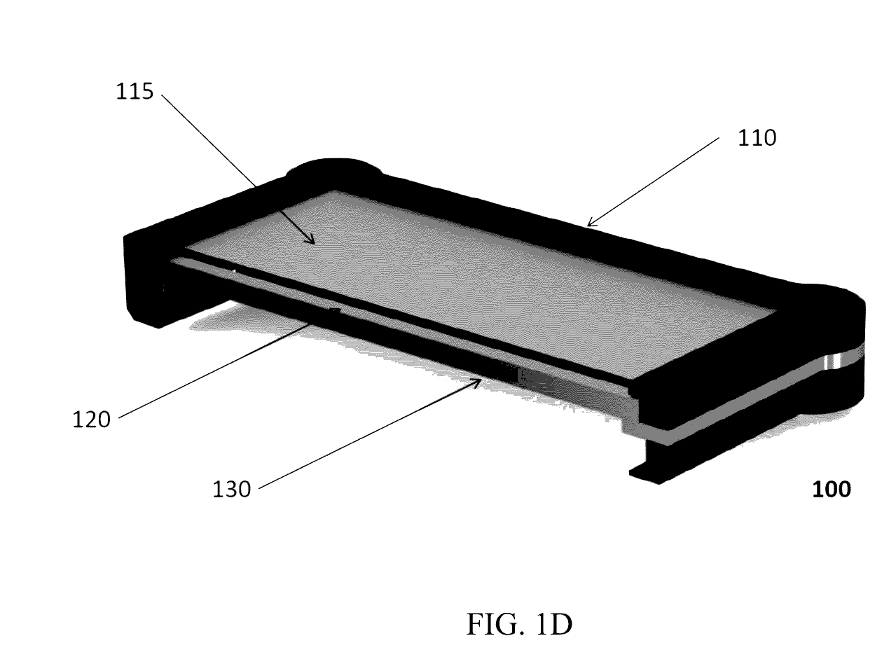

Turning now to the figures, FIG. 1A shows an example of a scale with a user display consistent with various aspects of the present disclosure. The apparatus includes a platform 101 and a user display 102. The user, as illustrated by FIG. 1A is standing on the platform 101 of the apparatus. The user display 102 is arranged with the platform 101. The user display 102 displays a FUI with data to the user. The FUI allows the user to interact with the scale. For instance, to interact with the scale, the user moves their foot, contacts the user display 102 with their toe, and/or shifts their weight to interact with the scale, among other interaction. The interaction causes the FUI to change and/or causes the scale to perform an action. As the user is standing on the scale, for various physiologic measurements, the FUI allows the user to interact with the scale without getting off the scale and/or changing their posture significantly.

For example, the user display 102 is configured to display the FUI including data through at least a portion of the platform 101 whereon the user stands. The FUI receives inputs from the user's foot to allow the user to interact with the scale apparatus. The interaction can include the user moving their foot, the user contacting a specific portion of the user display with their foot, the user shifting weight, and a combination thereof. For example, in some embodiments, the user display 102 includes a touch screen. In response to the user touching the screen with their foot, the FUI undergoes a change in appearance. Alternatively and/or in addition, the user display can be integrated with motion sense circuitry and/or accelerometers. In some embodiments, the scale is configured with a haptic, capacitive or flexible pressure-sensing upper surface, the (upper surface/tapping) touching from or by the user is sensed in the region of the surface and processed according to conventional X-Y grid Signal processing in the logic circuitry/CPU that is within the scale. By using one or more of the accelerometers located within the scale at its corners, such user data entry is sensed by each such accelerometer so long as the user's toe, heel or foot pressure associated with each tap provides sufficient force. In such embodiments, the interaction includes the user moving their foot, such as swiping their foot across the user display. The user may or may not touch the screen and the scale senses the movement as input.

In specific embodiments, the FUI displays a variety of text or images for the user to interact with the scale. For example, a variety of icons, a virtual keyboard, a listing of items, etc., can be displayed. In a specific embodiment, the FUI receives a user input in response to the user moving their foot to select an icon or item listed that is displayed using the FUI on the user display 102. The FUI displays, for example, a variety of icons that each represent a function (e.g., a test, a parameter, an action) that the scale can perform. In response to the user touching one of the icons with their foot, such as with their toe, the FUI revises the display to verify the selection. For example, the FUI displays text asking “Did you want to perform test A?” and include two icons listing “yes” or “no”. In response to the user selecting yes, the scale performs the test or other function. In response to the user selecting no, the scale redisplays the variety of icons for the user to re-select.

In other related embodiments, the FUI receives a user input in response to the user moving their foot on a virtual keyboard is displayed using the FUI on the user display 102. The user may type a command or an answer to a question to the scale, as discussed further herein.

As illustrated by the dashed-lines of FIG. 1A, the apparatus further includes processing circuitry 104, data-procurement circuitry 138, and physiologic sensors 108. That is, the dashed-lines illustrate a closer view of components of the apparatus. The physiologic sensors 108, in various embodiments, include a plurality of electrodes and force sensor circuitry 139 integrated with the platform 101. The electrodes and corresponding force sensor circuitry 139 are configured to engage the user with electrical signals and to collect signals indicative of the user's identity and cardio-physiological measurements while the user is standing on the platform 101. For example, the signals are indicative of physiological parameters of the user and/or are indicative of or include physiologic data, such as data indicative of a BCG or ECG and/or actual body weight or heart rate data, among other data. Although the embodiment of FIG. 1a illustrates the force sensor circuitry 139 as separate from the physiological sensors 108, one of skill in the art may appreciate that the force sensor circuitry 139 are physiological sensors. The user display 102 is arranged with the platform 101 and physiological sensors 108 to output user-specific information for the user while the user is standing on the platform 101. The processing circuitry 104 includes CPU and a memory circuit with user-corresponding data 103 stored in the memory circuit. The processing circuitry 104 is arranged under the platform 101 upon which the user stands, and is electrically integrated with the force sensor circuitry 139 and the plurality of electrodes (e.g., the physiologic sensors 108). The data indicative of the identity of the user includes, in various embodiments, user-corresponding data, biometric data obtained using the electrodes and/or force sensor circuitry, voice recognition data, images of the user, input from a user's device, and/or a combination thereof, and as discussed in further detail herein. The user-corresponding data includes information about the user that may or may not be obtained using the physiologic sensors 108, such as demographic information or historical information. Example user-corresponding data includes height, gender, age, ethnicity, exercise habits, eating habits, cholesterol levels, previous health conditions or treatments, family medical history, and/or a historical record of variations in one or more of the listed data. The user-corresponding data is obtained directly from the user (e.g., the user inputs to the scale) and/or from another circuit (e.g., a smart device, such a cellular telephone, smart watch and/or fitness device, cloud system, etc.). For example, the user-corresponding data is obtained and/or stored in a patient profile (e.g., patient profile database 123). The user-corresponding data 103 can be input and/or received prior to the user standing on the scale and/or in response to.

In various embodiments, the processing circuitry 104 is electrically integrated with the force sensor circuitry 139 and the plurality of electrodes and configured to process data obtained by the data-procurement circuitry 138 while the user is standing on the platform 101. The processing circuitry 104, for example, generates cardio-related physiologic data corresponding to the collected signals and that is manifested as user data. Further, the processing circuitry 104 generates data indicative of the identity of the user, such as a user ID and/or other user identification metadata. The user ID can be, for example, in response to confirming identification of the user using the collected signals indicative of the user's identity. The signals collected are indicative of physiologic parameters of the user and/or are indicative of or include physiologic data, such as data indicative of a BCG or ECG and/or actual body weight or heart rate data, among other data. As discussed further below, the signals can be force signals. The user display 102 is arranged with the platform 101 and the electrodes to output user-specific information for the user while the user is standing on the platform 101. The processing circuitry 104 includes CPU and a memory circuit with user-corresponding data 103 stored in the memory circuit. The processing circuitry 104 is arranged under the platform 101 upon which the user stands, and is electrically integrated with the force sensor circuitry 139 and the plurality of electrodes (e.g., the physiologic sensors 188). The data indicative of the identity of the user includes, in various embodiments, user-corresponding data, biometric data obtained using the electrodes and/or force sensor circuitry (or external circuitry), voice recognition data, images of the user, input from a user's device, and/or a combination thereof and as discussed in further detail herein. For example, the scale can capture voice sounds from the user speaking, and the user data indicative of the identity includes the voice sounds captured.

The user data, in some embodiments, includes the raw signals, bodyweight, body mass index, heart rate, body-fat percentage, cardiovascular age, among other data. Alternatively and/or in addition, the user data collected by the scale, in accordance with various embodiments, includes force signals, PWV, weight, heartrate, BCG, balance, tremors, respiration, data indicative of one or more of the proceeding data, and/or a combination thereof. In some embodiments, the user data includes the raw force signals and additional physiologic parameter data is determined using external circuitry. Alternatively, the physiologic data can include physiologic parameters such as the PWV, BCG, IPG, ECG that are determined using the force signals from the electrodes and the external circuitry (or the processing circuitry 104 of the scale) can determine additional physiologic parameters (such as determining the PWV using the BCG) and/or assess the user for a condition or treatment using the physiologic parameter. An algorithm to determine the physiologic data from raw signals can be located on the scale, on another device (e.g., external circuitry, cellphone), and on a Cloud system. For example, the Cloud system can learn to optimize the determination and program the scale to subsequently perform the determination locally. The Cloud system can perform the optimization and programming for each user of the scale.

In some embodiments, the scale collects physiologic data from other devices, such as medical devices, user devices, wearable devices, and/or remote-physiological devices. The data can include glucose measurements, blood pressure, ECG or other cardio-related data, body temperature, among other physiologic data. Further, the scale can act as a hub to collect data from a variety of sources. The sources includes the above-noted user devices. The scale can incorporate a web server (URL) that allows secure, remote access to the collected data. For example, the secure access can be used to provide further analysis and/or services to the user.

As used herein, a user device includes processing circuitry and output circuitry to collect various data (e.g., signals) and communicate the data to the scale and/or other circuitry. Example user devices include cellphones, tablets, standalone servers or CPUs, among other devices. A wearable device is a user device (and/or a remote user-physiologic device) that is worn by a user, such as on a user's wrist, head, or chest. Example wearable devices include smartwatches and fitness bands, smart glasses, chest heart monitors, etc. A remote user-physiologic device is a user device (and/or a wearable device) that further includes sensor circuitry or other circuit to collect physiologic data from the user, and, can optionally be in secured communication with the scale or other circuitry. Example remote user-physiological devices include smartwatches or fitness bands that collect heart rate and/or ECG and/or body temperature, medical devices, implanted medical devices, smartbeds, among other devices. Example physiologic data collected by remote user-physiologic devices includes glucose measurements, blood pressure, ECG or other cardio-related data, body temperature, among other data. As used herein, the terms “user device”, “wearable device”, and “remote user-physiologic device” can be interchangeably used, as one of skill may appreciate that in specific examples, a particular device may be considered one or more of a user device, a wearable device, a remote user-physiologic device. As a specific example, a particular remote user-physiologic device is a smartwatch and can be referred to as a wearable device or a user device. In other aspects, the remote user physiologic device may not be a wearable device, such as a medical device that is periodically or temporarily used.

In various embodiments, the processing circuitry 104, with the user display 102, displays at least a portion of the user data to the user. For example, user data that is not-regulated is displayed to the user, such as user weight. Alternatively and/or in addition, the user data is stored. For example, the user data is stored on the memory circuit of the processing circuitry 104 (e.g., such as the physiological user-data database 107 illustrated by FIG. 1a). The processing circuitry 104, in various embodiments, correlates the collected user data (e.g., physiologic user-data) with user-corresponding data, such as storing identification metadata that identifies the user with the respective data.

In a number of embodiments, the processing circuitry 104 and/or the scale includes an output circuit 106. The output circuit 106 receives the user data and, in response, output data from the processing circuitry to a user interface. The user interface is or includes a graphical user interface (GUI), FUI, and/or voice input/output circuitry. The user interface can be integrated with the platform 101 (e.g., internal to the scale) and/or can be integrated with external circuitry that is not located under the platform 101. In some embodiments, the user interface is a plurality of user interfaces, in which at least one user interface is integrated with the platform 101 and at least one user interface is not integrated with the platform 101. Example user interfaces include input/output devices, such as display screens, touch screens, microphones, etc.

A FUI is a user interface that allows for the user to interact with the scale via inputs using their foot and/or via graphic icons or visual indicators near the user's foot while standing on the platform. In specific aspects, the FUI receives inputs from the user's foot (e.g., via the platform) to allow the user to interact with the scale. The user interaction includes the user moving their foot relative to the FUI, the user contacting a specific portion of the user display, etc. The FUI can be arranged with the platform 101 and the data-procurement circuitry. The FUI outputs user-specific information to the user while the user is standing on the platform and allows the user to interact with the scale. For instance, to interact with the scale, the user moves their foot, contacts the user display 102 with their toe, and/or shifts their weight to interact with the scale, among other interaction. The interaction causes the FUI to change and/or causes the scale to perform an action. As the user is standing on the scale, the FUI allows the user to interact with the scale without getting off the scale and/or changing their posture significantly.

A GUI is a user interface that allows the user to interact with the scale through graphical icons and visual indicators. As an example, the external circuitry includes a GUI, processing circuitry, and output circuitry to communicate with the processing circuitry of the scale. The communication can include a wireless or wired communication. Example external circuitry can include a wired or wireless tablet, a cellphone (e.g., with an application), a smartwatch or fitness band, smart glasses, a laptop computer, among other devices. In other examples, the scale includes a GUI and voice input/output circuitry (as further described below) integrated in the platform 101. The user interact with the scale via graphical icons and visual indicators provided via the GUI and voice commands from the user to the scale.

Voice input/output circuitry (also sometimes referred to as speech input/output) can include a speaker, a microphone, processing circuitry, and other optional circuitry. The speaker outputs computer-generated speech (e.g., synthetic speech, instructions, and messages) and/or other sounds (e.g., alerts, noise, recordings, etc.) The computer-generated speech can be predetermined, such as recorded messages, and/or can be based on a text-to-speech synthesis that generates speech from computer data. The microphone captures audio, such a voice commands from the user and produces a computer-readable signal from the audio. For example, the voice input/output circuitry can include an analog-to-digital converter (ADC) that translates the analog waves captured by the microphone (from voice sounds) to digital data. The digital data can be filtered using filter circuitry to remove unwanted noise and/or normalize the captured audio. The processing circuitry (which can include or be a component of the processing circuitry 104) translates the digital data to computer commands using various speech recognition techniques (e.g., pattern matching, pattern and feature matching, language modeling and statistical analysis, and artificial neural networks, among other techniques).

In specific embodiments, the output circuitry 106 provides the user data to the user display 102 for display by a FUI 108 and for viewing by the user through the platform. In various embodiments, the output circuit 106 displays on the user display 102 the user's weight and the data indicative of the user's identity and/or the generated cardio-related physiologic data corresponding the collected signals. In some embodiments, the output circuit 106 sends the user data, including the data indicative of the user's identity and the generated cardio-related physiologic data, from the scale for reception at external circuitry that is not integrated within the scale. The communication, in various embodiments, includes a wireless communication and/or utilizes a cloud system.

In a number of embodiments, the FUI is used to provide (e.g., display) alerts correlated to user data (e.g., scale-obtained data of the user). Such alerts includes notifications of social groupings available for the user that is tailored to the user data, additional information regarding social groupings and/or health information, and/or an indication that additional information is available for the user on an standalone CPU in communication with the platform apparatus, such as a smartphone, tablet and/or computing device. The additional information includes various advertisements, such as products and services available and correlated with the scale-obtained data, generic health information, updates on the social groupings, available diagnosis information from a physician, requests for participation in studies by a physician and/or other research, among other data.

Social groupings, as used herein, includes grouping of a set of scale users based on scale-obtained data. In some embodiments, the social groups are intra scale. For example, the scale is configured to collect user data for two or more users and correlate the respective data with a user profile of each respective users. The social grouping of an intra scale includes grouping the users of the scale and providing various reports, updates, alerts, and/or forums for the users of the group to interact. The forum, in some embodiments, includes a private (or public) page of a social network webpage that the users of the group access and communicate. A private page, for instance, is only accessible by the users of the group and/or persons authorized by users of the group. In other embodiments, the social groupings are inter scale. For example, an external circuitry, such as a server CPU, may receive user data (with user identifying data removed) from a plurality of scales and identifies various users with correlated user data. The users with correlated user data, such as demographic data and/or scale-obtained data, are grouped by the external circuitry without user input. The external circuitry outputs an indication of an available social group to the scales of the users with the correlated user data and each scale displays, using the FUI, an alert of an available social group. The user accesses the social grouping using the FUI and/or a standalone CPU that is in communication with the scale. For example, in response to an alert, the user selects an interest in the social grouping using the FUI. The scale outputs the indication and a link to a webpage or application associated with the social group (or information on how to access the social grouping) the standalone CPU, such as a user smartphone or tablet. The webpage includes, in some embodiments, a page of a social network, an application or portal for the user to log-in to, a forum, etc. In various embodiments, data is tracked for users of the social group and reports are provided, such as rankings of the users in the group, progress of the users, new observations, and/or information learned. Alternatively and/or in addition, the users of the group are provided a forum to discuss various health issues, successes, failures, exercise, eating, etc.

As a specific example, a scale is used by a family training for a marathon. Each member of the family uses the scale to track various physiological parameters, include cardiogram related characteristics, recovery parameters, weight, body-mass-index, and exercise results. The family is grouped into an intra scale social grouping and provided with alerts when reports of progress and/or rankings are available for the family. In another specific example, multiple scales are used by different users located at different locations that have indicators for atrial fibrillation, are female, are over-weight, and are over the age of sixty-years old. The users are grouped into an inter scale social grouping and provided with an alert of an available social grouping. In response to at least a subset of the users selecting an interest in the social grouping, the subset of users are provided with a link to a webpage, portal, application, and/or forum. The subset of users access the link and are connected one another. In various embodiments, user data (with user identifying data removed) is displayed to the social group so that users can view other users' success and/or failures.

In various embodiments, the user display 102 and the FUI are used to verify the user's identity. For instance, the processing circuitry 104 validates the user data as concerning a user associated with a specific user profile using the data indicative of the user's identity. The validation, in some embodiments, includes comparing user data to a user profile. In various embodiments, the data indicative of the user's identity is a user ID and/or is associated with the user ID (e.g., is mapped to and/or otherwise correlated to). In response to the identification, the processing circuitry 104 outputs the identification to the output circuit 106 for display on the user display 102. The FUI displays data to confirm identification of the user and, in response to a user input, outputs the confirmation to the processing circuitry 104 to authorize identification of the user. For example, the FUI displays the identification of the user and asks the user to verify the identification. In response to the verification by the user by a foot-based user interaction with the FUI, the output circuit 106 outputs the verification to the processing circuitry 104. In a specific example, when the user stands on the platform of the scale, and the scale detects touching of the toe, the scale can reject the toe touch (or tap) as a foot signal (e.g., similar to wrist rejection for capacitive tablets with stylus).

In various embodiments, the identification is based on a scale-obtained biometric. The scale-obtained biometric is learned by the scale, in some embodiments, during an initialization mode of the scale. For example, in various embodiments, a user is correlated with a user profile stored on the scale and/or otherwise accessible by the scale. The user profile is set up during the initialization mode and/or includes an indication of a scale-based biometric. For example, a user without a user profile set-up steps onto the scale. The scale is unable to identify the user using collected signals as the user does not have an indication of a scale-obtained biometric stored and/or accessible by the scale. The scale operates in a default mode by displaying the user's body mass and/or weight using the FUI and does not output user data to any external circuitry. The scale, in various embodiments, displays a prompt (e.g., an icon) on the FUI indicating the user can establish a user profile. In response to the user selecting the prompt, the scale, using the FUI, enters an initialization mode. During the initialization mode, the scale asks the users various questions, such as identification of external circuitry to send data to, identification information of the first user, and/or demographics of the user. The user provides inputs using the FUI to establish scale-based biometrics to enable one or more communication modes that are associated with the user profile. The scale further collects user data to identify the scale-based biometrics and stores an indication of the scale-based biometric in the user profile such that during subsequent measurements, the scale recognizes the user and authorizes a particular communication mode. Alternatively, the user provides inputs using another device that is external to the scale and is in communication with the scale (e.g., a cellphone).

In various embodiments, the scale is configured to collect data for two or more users and correlates the user data with respective user profiles. As previously discussed, the scale recognizes each of the two or more users based on a scale-based biometric. In various embodiments, the user profiles are associated with a hierarchy of different levels of biometrics that enable different data to be communicated and/or to different sources. For example, in response to verifying a first biometric, the scale outputs the user's weight to the user's smartphone or other standalone CPU. The user, using the FUI and/or another GUI that is in communication with the scale, configures the scale with a second biometric to output user data to external circuitry and/or that is more user-sensitive, as discussed further herein. In response to verifying the second biometric, the scale outputs the user data (such as higher-sensitivity user data) from the scale to the smartphone or standalone CPU, from the scale to the smartphone/standalone CPU for sending to a third party, and/or from the scale to the third party.

The user profile stores historical user data, including generated cardio-related physiologic data. Further, the user profile includes various stored user preferences. For example, the FUI, in response to verification of the user's identity, is revised based on user preferences of the user associated with the user profile. The validation, as previously discussed, can be performed using the collected signals indicative of the user's identity and/or in response to verification by a user interaction with the FUI. The user preferences include text or image size, data to display using the FUI, commonly access test or features, specific parameters to track, among other settings.

In a number of embodiments, the user interaction changes the view of the FUI. The change can be, for example, by changing user preferences stored in the user profile and/or based on user interaction. For example, the user may be unable to read the data displayed using the FUI. The user, using their foot, revises the size of the data. For example, the FUI displays an icon on the side of the user display 102 that the user can select to increase or decrease text or image size. The icon may include a sliding bar that the user contacts with their foot and moves. Alternatively, the user can tap their foot on the FUI to change the text or image size.

The font size and/or configuration, in various embodiments, of text and/or images displayed using the FUI is automatically configured based on user demographic information and/or the data to be displayed. In some embodiments, the scale includes a display configuration filter (e.g., circuitry and/or computer readable medium) configured to discern the data to display to the user. The display configuration filter discerns which portions of the user data, clinical indications, and/or other information to display to the user using the FUI of the scale based on various user demographic information (e.g., age, gender, height, diagnosis) and the amount of data. For example, the data may include an amount of data that if all the data is displayed on the FUI, the data is difficult for a person to read and/or uses multiple display screens.

The display configuration filter discerns portions of the data to display using the FUI based on the data and the demographic information, and discerns other portions of the data to display on another user device. The other user device is selected by the scale (e.g., the filter) based on various communications settings. The communication settings include settings such as user settings (e.g., the user identifying user devices to output data to), scale-based biometrics (e.g., user configures scale, or default settings, to output data to user devices in response to identifying scale-based biometrics), and/or proximity of the user device (e.g., the scale outputs data to the closest user device among a plurality of user devices and/or in response to the user device being within a threshold distance from the scale), among other settings. For example, the scale determines which portions of the information to output to the other particular user device based on user settings/communication authorization (e.g., what user devices are authorized by the user to receive particular user data from the scale), and proximity of the user device to the scale. The determination of which portions to output is based on what type of data is being displayed, how much data is available, and the various user demographic information (e.g., an eighteen year old is able to see better than a fifty year old).

For example, in some specific embodiments, the scale operates in different modes of data security and communication. The different modes of data security and communication are enabled in response to biometrics identified by the user and using the FUI. In some embodiments, the scale is used by multiple users and/or the scale operates in different modes of data security and communication in response to identifying the user and based on biometrics. The different modes of data security and communication include, for example: a first mode (e.g., default mode) in which the user's body mass and/or weight is displayed regardless of any biometric which would associate with the specific user standing on the scale and no data is communicated to external circuitry; a second mode in which complicated/more-sensitive data (or data reviewed infrequently) is only exported from the scale under specific manual commands provided to the scale under specific protocols and in response to a biometric; and third mode or modes in which the user-specific data that is collected from the scale is processed and accessed based on the type of data and in response to a biometric. Such data categories include categories of different levels of importance and/or sensitivities such as the above-discussed high and low level data and other data that might be very specific to a symptom and/or degrees of likelihood for diagnoses. Optionally, the CPU in the scale is also configured to provide encryption of various levels of the user's sensitive data.

In some embodiments, the different modes of data security and communication are enabled in response to recognizing the user standing on the scale using a biometric and operating in a particular mode of data security and communication based on user preferences and/or services activated. For example, the different modes of operation include the default mode (as discussed above) in which certain data (e.g., categories of interest, categories of user-sensitive user data, or historical user data) is not communicated from the scale to external circuitry, a first communication mode in which data is communicated to external circuitry as identified in a user profile, a second or more communication modes in which data is communicated to a different external circuitry for further processing. The different communication modes are enabled based on biometrics identified from the user and user settings in a user profile corresponding with each user.

In a specific embodiment, a first user of the scale may not be identified and/or have a user profile set up. In response to the first user standing on the scale, the scale operates in a default mode. During the default mode, the scale displays the user's body mass and/or weight on the user display and does not output user data. The scale, in various embodiments, displays a prompt (e.g., an icon) on the FUI indicating the first user can establish a user profile, such as during an initialization mode as previously discussed.

A second user of the scale has a user profile set up that indicates the user would like data communicated to a computing device of the user. When the second user stands on the scale, the scale recognizes the second user based on a biometric and operates in a first communication mode. During the first communication mode, the scale outputs at least a portion of the user data to an identified external circuitry. For example, the first communication mode allows the user to upload data from the scale to a user identified external circuitry (e.g., the computing device of the user). The information may include user data and/or user information that has low-user sensitivity, such as user weight and/or bmi. In the first communication mode, the scale performs the processing of the raw sensor data and/or the external circuitry can. For example, the scale sends the raw sensor data and/or additional health information to a user device of the user. The computing device may not provide access to the raw sensor data to the user and/or can send the raw sensor data to another external circuitry for further processing in response to a user input. For example, the computing device can ask the user if the user would like generic health information and/or regulated health information as a service. In response to receiving an indication the user would like the generic health information and/or regulated health information, the computing device outputs the raw sensor data and/or non-regulated health information to another external circuitry for processing, providing to a physician for review, and controlling access, as discussed above.

In one or more additional communication modes, the scale outputs raw sensor data to an external circuitry for further processing. For example, during a second communication mode and a third communication, the scale sends the raw sensor data and/or other data to external circuitry for processing. Using the above-provided example, a third user of the scale has a user profile set up that indicates the third user would like scale-obtained data to be communicated to an external circuitry for further processing, such as to determine generic health information. When the third user stands on the scale, the scale recognizes the third user based on one or more biometrics and operates in a second communication mode. During the second communication mode, the scale outputs raw sensor data to the external circuitry. The external circuitry identifies one or more risks, and, optionally, derives generic health information. In some embodiments, the external circuitry outputs the generic health information to the scale. The scale, in some embodiments, displays a synopsis of the generic health information and/or outputs a full version of the generic health information to another user device for display (such as, using the filter described above) and/or an indication that generic health information can be accessed.

A fourth user of the scale has a user profile set up that indicates the fourth user has enabled a service to access regulated health information. When the fourth user stands on the scale, the scale recognizes the user based on one or more biometrics and operates in a fourth communication mode. In the fourth communication mode, the scale outputs raw sensor data to the external circuitry, and the external circuitry processes the raw sensor data and controls access to the data. For example, the external circuitry may not allow access to the regulated health information until a physician reviews the information. In some embodiments, the external circuitry outputs data to the scale, in response to physician review. For example, the output data can include the regulated health information and/or an indication that regulated health information is ready for review. The external circuitry may be accessed by the user, using the scale and/or another user device. In some embodiments, using the FUI of the scale, the scale displays the regulated health information to the user. The scale, in some embodiments, displays a synopsis of the regulated health information (e.g., clinical indication) and outputs the full version of regulated health information to another user device for display (such as, using the filter described above) and/or an indication that the regulated health information can be accessed to the scale to display. In various embodiments, if the scale is unable to identify a particular (high security) biometric that enables the fourth communication mode, the scale may operate in a different communication mode and may still recognize the user. For example, the scale may operate in a default communication mode in which the user data collected by the scale is stored in a user profile corresponding to the fourth user and on the scale. In some related embodiments, the user data is output to the external circuitry at a different time.

Although the present embodiments illustrates a number of security and communication modes, embodiments in accordance with the present disclosure can include additional or fewer modes. Furthermore, embodiments are not limited to different modes based on different users. For example, a single user may enable different communication modes in response to particular biometrics of the user identified and/or based on user settings in a user profile. Although the different communications are referred to as “modes”, one of skill in the art may appreciate that the communications in the different modes may not (or may) include different media and channels. The different communication modes can include different devices communicated to and/or different data that is communicated based on sensitivity of the data and/or security of the devices.

In various embodiments, the scale defines a user data table that defines types of user data and sensitivity values of each type of user data. In specific embodiments, the FUI displays the user data table. In other specific embodiments a user interface of a smartphone, tablet, and/or other computing device displays the user data table. For example, a wired or wireless tablet is used, in some embodiments, to display the user data table. The sensitivity values of each type of user data, in some embodiments, define in which communication mode(s) the data type is communicated and/or which biometric is used to enable communication of the data type. In some embodiments, a default or pre-set user data table is displayed and the user revises the user data table using the FUI. The revisions are in response to user inputs using the user's foot and/or contacting or moving relative to the FUI. Although the embodiments are not so limited, the above (and below) described control and display is provided using a wireless or wired tablet or other computing device as a user interface. The output to the wireless or wired tablet, as well as additional external circuitry, is enabled using biometrics. For example, the user is encouraged, in particular embodiments, to configure the scale with various biometrics. The biometric include scale-based biometrics and biometrics from the tablet or other user computing device. The biometric, in some embodiments, used to enable output of data to the tablet and/or other external circuitry includes a higher integrity biometric (e.g., higher likelihood of identifying the user accurately) than a biometric used to identify the user and stored data on the scale. For example, the scale-based biometrics can include a high security biometric and a low security biometric. An example high security biometric can include an ECG-to-BCG timing relationship in addition to (or on its own) one or more of a foot shape, toe tapped password and/or a toe print. A low security biometric can include a user weight, a foot size, a body-mass-index. In response to identifying the low security biometric, the scale operates in a low verified communication mode.

An example user data table is illustrated below:

| | | Body | | | |
|---|---|---|---|---|---|
| | | Mass | | | |
| | | Index, | User- | Physician- | Scale-stored |
| | Weight, | user | Specific | Provided | suggestions |
| User-data | local | specific | Advertise- | Diagnosis/ | (symptoms & |
| Type | weather | news | ments | Reports | diagnosis) |
| Sensitivity | 1 | 3 | 5 | 10 | 9 |
| (10 = | | | | | |
| highest, 1 = | | | | | |
| lowest) | | | | | |

The above-displayed table is for illustrative purposes and embodiments in accordance with the present disclosure can include additional user-data types than illustrated, such as cardiogram characteristics, clinical indications, physiological parameters, user goals, demographic information, etc. In various embodiments, the user data table includes additional rows than illustrated. The rows, in specific embodiments, include different data input sources and/or sub-data types (as discussed below). Data input sources include source of the data, such as physician provided, input from the Internet, user provided, from the external circuitry. The different data from the data input sources, in some embodiments, is used alone or in combination.

In accordance with various embodiments, the scale uses a cardiogram (on its own or in addition to weight, BCG, ECG, and/or various combinations) of the user and/or other scale-obtained biometrics to differentiate between two or more users. The scale-obtained data includes health data that is user-sensitive, such that unintentional disclosure of scale-obtained data is not desired. Differentiating between the two or more users and automatically communicating (e.g., without further user input) user data responsive to scale-obtained biometrics, in various embodiments, provides a user-friendly and simple way to communicate data from a scale while avoiding and/or mitigating unintentional (and/or without user consent) communication. For example, the scale, such as during an initialization mode for each of the two or more users and as previously discussed, collects user data to identify the scale-based biometrics and stores an indication of the scale-based biometrics in a user profile corresponding with the respective user. During subsequent measurements, the scale recognizes the particular user by comparing collected signals to the indication of the scale-based biometrics in the user profile. The scale, for example, compares the collected signals to each user profile of the two or more users and identifies a match between the collected signals and the indication of the scale-based biometrics. A match, in various embodiments, is within a range of values of the indication stored. Further, in response to verifying the scale-based biometric(s), a particular communication mode is authorized.

In accordance with a number of embodiments, the scale identifies one or more of the multiple users of the scale that have priority user data. The user data with a priority, as used herein, includes an importance of the user and/or the user data. In various embodiments, the importance of the user is based on parameter values identified and/or user goals, such as the user is an athlete and/or is using the scale to assist in training for an event (e.g., marathon) or is using the scale for other user goals (e.g., a weight loss program). Further, the importance of the user data is based on parameters values and/or user input data indicating a diagnosis of a condition or disease and/or a risk of the user having the condition or disease based on the scale-obtained data. For example, the scale-obtained data of a first user indicates that the user is overweight, recently had an increase in weight, and has a risk of having atrial fibrillation. The first user is identified as a user corresponding with priority user data. A second user of the scale has scale-obtained data indicating a decrease in recovery parameters (e.g., time to return to baseline parameters) and the user inputs an indication that they are training for a marathon. The second user is also identified as a user corresponding with priority user data. The scale displays indications to user with the priority user data, in some embodiments, on how to use to the scale to communicate the user data to external circuitry for further processing, correlation, and/or other features, such as social network connections. Further, the scale, in response to the priority, displays various feedback to the user, such as user-targeted advertisements and/or suggestions. In some embodiments, only users with priority user data have data output to the external circuitry to determine risks, although embodiments in accordance with the present disclosure are not so limited.

In some embodiments, one or more users of the scale have multiple different scale-obtained biometrics used to authorize different communication modes. The different scale-obtained biometrics are used to authorize communication of different levels of user sensitive data, such as the different user-data types and sensitivity values as illustrated in the above-table. For example, in some specific embodiments, the different scale-obtained biometrics include a high security biometric, a medium security biometric, and a low security biometric. Using the above illustrated table as an example, the three different biometrics are used to authorize communication of the user-data types of the different sensitivity values. For instance, the high security biometric authorizes communication of user-data types with sensitivity values of 8-10, the medium security biometric authorizes communication of user-data types with sensitivity values of 4-7, and the low security biometric authorizes communication of user-data types with sensitivity values of 1-3. The user, in some embodiments, can adjust the setting of the various biometrics and authorization of user-data types.

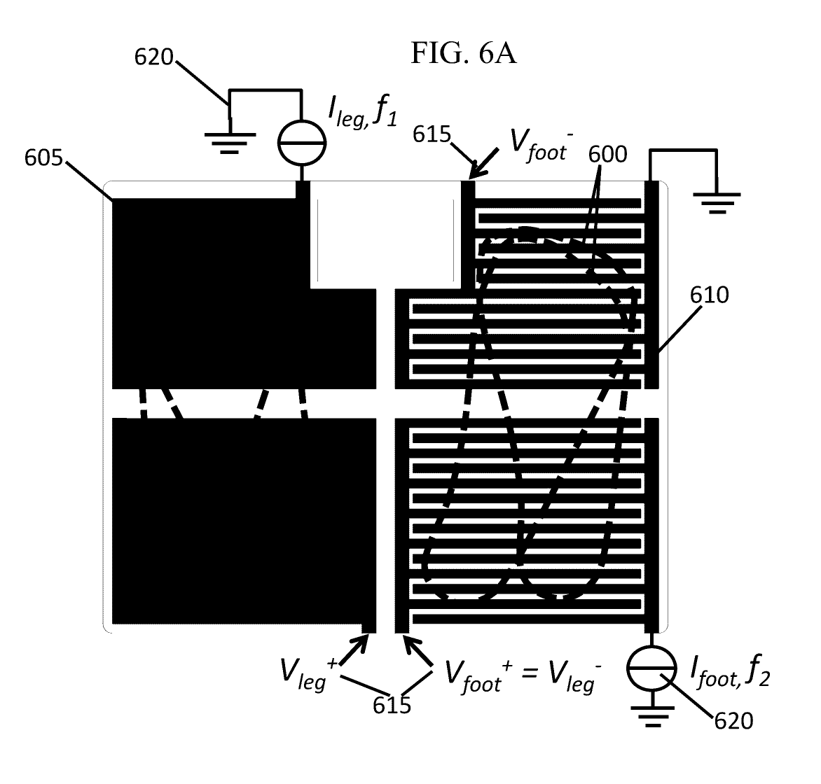

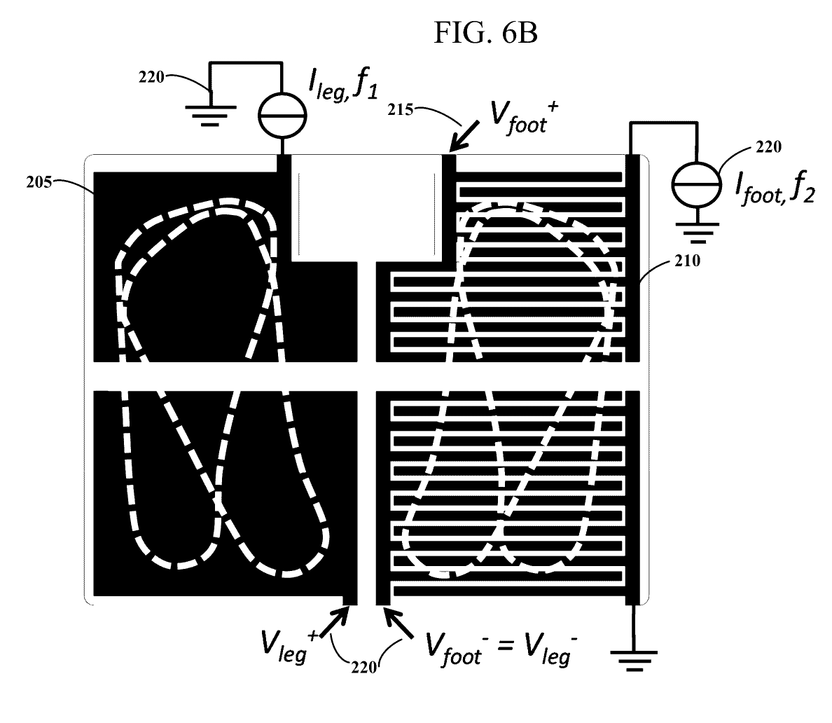

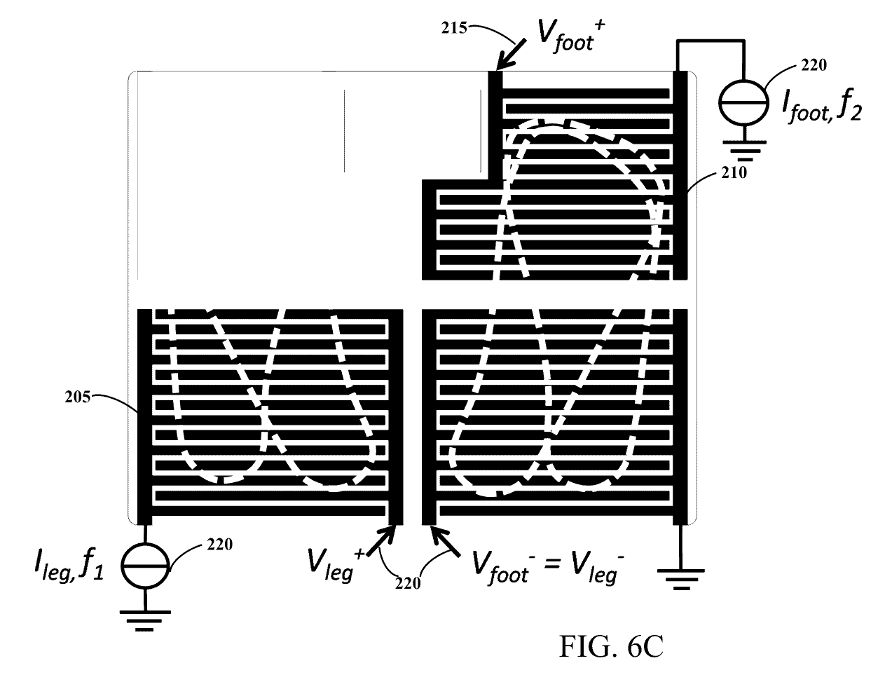

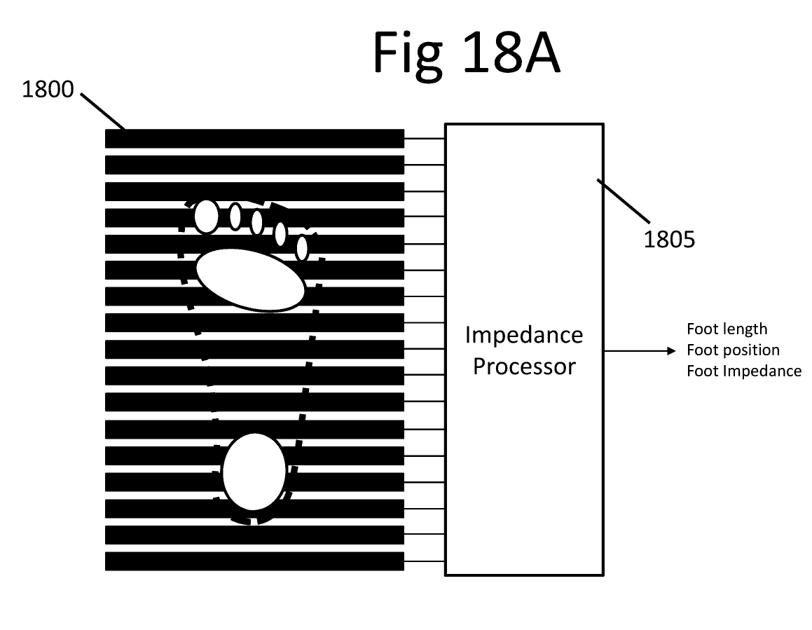

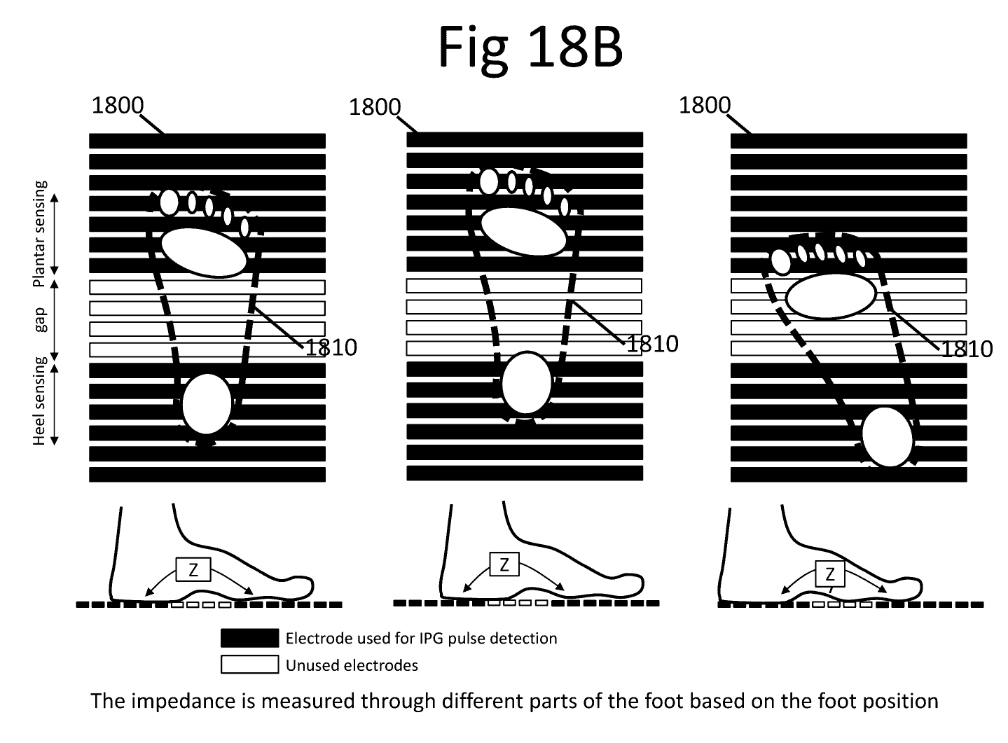

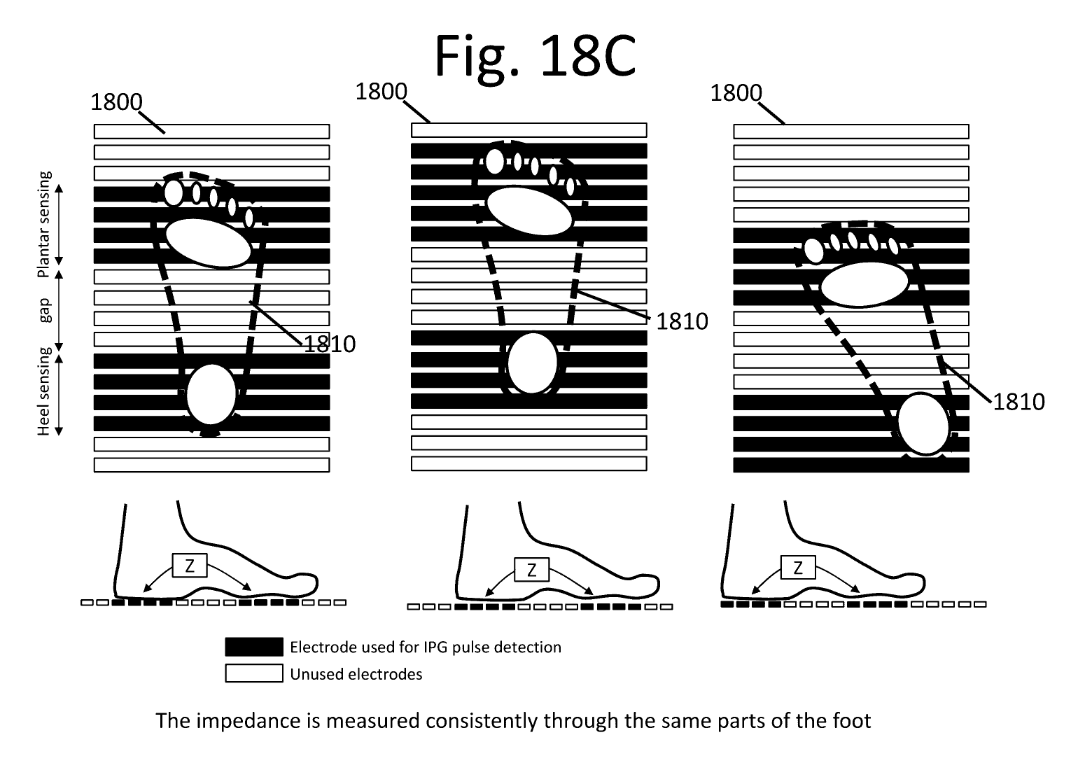

In a specific example, low security biometrics includes estimated weight (e.g., a weight range), and a toe tap on the FUI Example medium security biometrics includes one or more the low security biometric in addition to length and/or width of the user's foot, and/or a time of day or location of the scale. For example, as illustrated by FIGS. 6A and 18A-18C, the scale includes impedance electrodes that are interleaved and engage the feet of the user. The interleaved electrodes assist in providing measurement results that are indicative of the foot length, foot width, and type of arch. Further, a specific user, in some embodiments, may use the scale at a particular time of the day and/or authorize communication of data at the particular time of the day, which is used to verify identity of the user and authorize the communication. The location of scale, in some embodiments, is based on Global Positioning System (GPS) coordinates and/or a Wi-Fi code. For example, if the scale is moved to a new house, the Wi-Fi code used to communicate data externally from the scale changes. Example high security biometrics include one or more low security biometrics and/or medium security biometrics in addition to cardiogram characteristics and, optionally, a time of day and/or heart rate. Example cardiogram characteristics include a QRS complex, and QRS complex and P/T wave, BCG wave characteristics, ECG-to-BCG timing, and combinations thereof.

In various embodiments, the user adjusts the table displayed above to revise the sensitivity values of each data type. Further, although the above-illustrated table includes a single sensitivity value for each data type, in various embodiments, one or more of the data types are separated into sub-data types and each sub-data type has a sensitivity value. As an example, the user-specific advertisement is separated into: prescription advertisement, external device advertisements, exercise advertisements, and diet plan advertisement. Alternatively and/or in addition, the sub-data types for user-specific advertisement include generic advertisements based on a demographic of the user and advertisements in response to scale collected data (e.g., advertisement for a device in response to physiologic parameters), as discussed further herein.

For example, weight data includes the user's weight and historical weight as collected by the scale. In some embodiments, weight data includes historical trends of the user's weight and correlates to dietary information and/or exercise information, among other user data. Body mass index data, includes the user's body mass index as determined using the user's weight collected by the scale and height. In some embodiments, similar to weight, body mass index data includes history trends of the user's body mass index and correlates to various other user data.

User-specific advertisement data includes various prescriptions, exercise plans, dietary plans, and/or other user devices and/or sensors for purchase, among other advertisements. The user-specific advertisements, in various embodiments, are correlated to input user data and/or scale-obtained data. For example, the advertisements include generic advertisements that are relevant to the user based on a demographic of the user. Further, the advertisements include advertisements that are responsive to scale collected data (e.g., physiological parameter includes a symptom or problem and advertisement is correlated to the symptom or problem). A number of specific examples include advertisements for beta blockers to slow heart rate, advertisements for a user wearable device (e.g., Fitbit®) to monitor heart rate, and advertisements for a marathon exercise program (such as in response to an indication the user is training for a marathon), etc.

Physician provided diagnosis/report data includes data provided by a physician and, in various embodiments, is in responsive to the physician reviewing the scale-obtained data. For example, the physician provided diagnosis/report data includes diagnosis of a disorder/condition by a physician, prescription medication prescribed by a physician, and/or reports of progress by a physician, among other data. In various embodiments, the physician provided diagnosis/reports are provided to the scale from external circuitry, which includes and/or accesses a medical profile of the user.

Suggestion data includes data that provides suggestions or advice for symptoms, diagnosis, and/or user goals. For example, the suggestions include advice for training that is user specific (e.g., exercise program based on user age, weight, and cardiogram data or exercise program for training for an event or reducing time to complete an event, such as a marathon), suggestions for reducing symptoms including dietary, exercise, and sleep advice, and/or suggestions to see a physician, among other suggestions. Further, the suggestions or advice include reminders regarding prescriptions. For example, based on physician provided diagnosis/report data and/or user inputs, the scale identifies the user is taking a prescription medication. The identification includes the amount and timing of when the user takes the medication, in some embodiments. The scale reminds the user and/or asks for verification of consumption of the prescription medication using the FUI.

As further specific examples, recent discoveries may align and associate different attributes of scale-based user data collected by the scale to different tools, advertisements, and physician provided diagnosis. For example, it has recently been discovered that atrial fibrillation is more directly correlated with obesity. The scale collects various user data and monitors weight and various components/symptoms of atrial fibrillation. In a specific embodiment, the scale recommends/suggests to the user to: closely monitor weight, recommends a diet, goals for losing weight, and correlates weight gain and losses for movement in cardiogram data relative to arrhythmia. The movement in cardiogram data relative to arrhythmia, in specific embodiments, is related to atrial fibrillation. For example, atrial fibrillation is associated with indiscernible p-waves and beat to beat fluctuations. Thereby, the scale correlates weight gain/loss with changes in amplitude (e.g., discernibility) of a p-wave of a cardiogram (preceding a QRS complex) and changes in beat to beat fluctuations.

In various related embodiments, the scale is used to perform a question and answer session. For example, the scale can display questions using the user display 102 and FUI. The user can answer the questions by the FUI displaying pre-set answers as icons or in a list and/or using a virtual keyboard displayed using the FUI. The questions are used to identify symptoms and/or reasons why the user is visiting the physician. The answers input by the user are used to update the user profile. The update, for instance, includes populating the data in the specific user profile. In various embodiments, the scale and/or other external circuitry uses the input data to determine clinical indication data and/or to further refine stored clinical indication data, as discussed further herein. As a specific embodiment, the scale engages the user with electrical signals and generates cardio-related physiologic data therefrom. The cardio-related physiologic data indicates that the user may have a heart condition. In response, the FUI displays a plurality of questions to the user. The plurality of questions include various symptoms associated with the heart condition. For each question, the FUI displays a set of icons or a list that contain answers to the questions. One of the icons or items in the list includes “answer not displayed”. If the user selects “answer not displayed” with their foot, a virtual keyboard is displayed for the user to type their answer using their foot. In response to the user providing an answer to the questions, the answers are stored within the user profile. In some embodiments, the user profile can be viewed by a physician for further medical analysis, as discussed further herein.

FIG. 1B shows an example process for controlling display of data using a FUI consistent with various aspects of the present disclosure. The apparatus illustrated by FIG. 1b can include the apparatus, including the platform 101 and user display 102, as previously illustrated and discussed with regard to FIG. 1a. As illustrated, the apparatus includes a platform, a user display 102, data-procurement circuitry 138, and processing circuitry 104. The data-procurement circuitry 138 includes force sensor circuitry and a plurality of electrodes (e.g., the physiologic sensors) which are integrated with the data-procurement circuitry 138. The processing circuitry 104 includes a CPU and a memory circuit with user-corresponding data stored in the memory circuit. As previously discussed, the user display 102 displays a FUI within at least a portion of the platform.

The FUI, as previously described, allows for the user to interact with the scale and displays data, such as an alert of available data, to the user. For example, as illustrated by FIG. 1B, the scale at block 116 is in a reduced power-consumption mode of operation and is waiting for a user to stand on the platform. In various embodiments, in response to the user standing on and/or approaching the scale, the scale transitions from the reduced power-consumption mode of operation to at least one higher power-consumption mode of operation at block 118. As illustrated, the higher power-consumption mode includes activating the FUI. In some embodiments, the user display is already activated during the reduced power-consumption mode of operation, but may be displaying a “screen saver.” The activation of the FUI, as used herein, includes displaying the FUI including data through at least a portion of the platform whereon the user stands and/or is about to stand. The display, upon the transition, in some embodiments, is used to verify identity of the user and/or an associated user profile, and/or to identity various measurements, or features to perform. For example, the scale may be associated with a limited number of users (e.g., three people living in a home) and the display can include icons with each of the number of users.

For example, optionally the display (which can be before or after collected signals) includes a list and/or icons containing the various measurements, features, or test to perform. The FUI, in some embodiments, displays a plurality of icons of a list that includes the plurality of measurements, test, and/or features. In response to user input to the FUI selecting one of the icons or item, the scale performs the selected measurement, test, or features. For example, in response to the user placing their foot on one of the plurality of measurements, tests, or features in the list, the scale performs the selection. In some related embodiments, the displayed measurements, tests, and/or features can be tailed to the specific user. For example, the scale identifies the user (using signals collected from the user and/or in response to user input) and identifies recently selected measurements, test, and/or features and/or user preferences that include measurements, test, and/or features.

In response to the user standing on the scale, at block 119, the scale engages the user with electrical signals, using the data-procurement circuitry, and collects signals indicative of the user's identity and/or cardio-physiological measurements (e.g., force signals) while the user is standing on the platform. The processing circuitry 104, at block 121, processes the signals to generate cardio-related physiologic data manifested as user data. In various embodiments, prior to collecting the signals, the FUI is used to display an instruction to the user to hold still during the engagement of the user with the electrical signals and the collection of signals. The instruction can indicate to stay still until an alert is provided and the scale can provide the alert after collecting the signals.

At block 122, the apparatus, using the processing circuitry 104, optionally confirms identification of the user when the user is standing on the platform. The identification, in various embodiments, is based on the signals indicative of the user's identity, such as a biometric collected using the scale, and/or user data. For example, the processing circuitry 104 compares the scale-obtained biometric to stored user-corresponding data to confirm identification of the user. Further, in some embodiments, the FUI is used to additionally verify the identity using a display. For example, in response to the verification using the collected signals, user input is received using the FUI and from the user's foot. The FUI, in some embodiments, displays the identity determined using the collected signals and the user input verifies the identity is correct. Responsive the user input, the scale validates the user data, including data indicative of the user's identity and the generated cardio-related physiologic data, as concerning a specific user associated with a user profile. That is, the FUI displays verification data to the user and in response to a user input to the scale can validate the user data. Further, the validated user data, at block 123, is used to update the user profile. For example the user data is stored within the user profile.

At block 126, data is displayed using the FUI. For example, the user's weight is displayed to the user. Additionally, the generated cardio-related physiologic data and/or other health information can be displayed. The scale, in various embodiments, discerns what data and/or amount of data to display. For example, the scale discerns what data to display based on user demographic information, what type of data is being display, and/or user-specific use of the scale. User-specific use of the scale includes data such as frequency of use, time of use (e.g., time of day) features used and/or enabled, priority of user data, among other information. In specific aspects, the time of day can be useful for obtaining PWV and heart rate. For example, the scale records user data and can prompt the user at set times. In various embodiments, a subset of the data, such as a synopsis of the data is displayed using the FUI and an indication that further information is available on another electronic device. The full data is sent to the other electronic device, such as a smartphone or other standalone CPU, and displayed using the other electronic device's user display. In some embodiments, the user-specific use of the scale includes previous data that the user accessed and how the user viewed the data.

For instance, the scale includes a FUI that has limited space. Based on the amount of data, the height of the user, and/or past user responses, the scale discerns whether to display the data on the FUI and/or output the data to another electronic device. For example, the scale displays a synopsis, a subset of the total data, and/or an indication of a data on the user display of the scale which includes an icon for the user to select to view more information. In response to the user selecting the icon, the scale outputs the data to the other electronic device, such as the user's smartphone and/or displays the advertisement on the user display. Alternatively, in response to the user selecting the icon, the scale displays another one or more icons for the user to select which device to display the data on. Based on the user inputs to the FUI overtime, the scale automatically displays data based on how the user responds in the past and/or over time. For example, if the user continuously displays the data on their smartphone, the scale outputs the data to the user's smartphone. By contrast, if the user does not indicate an interest in particular data, such as advertisements, the scale does not display the advertisements. The scale displays or outputs the data in response to verifying a scale-based biometric, in various embodiments.

Priority of user data, as used herein, includes an importance of the user and/or the user data. In various embodiments, the importance of the user is based on parameter values identified and/or user goals, such as the user is an athlete and/or is using the scale to assist in training for an event (e.g., marathon) or is using the scale for other user goals (e.g., a weight loss program). Further, the importance of the user data is based on parameters values and/or user input data indicating a diagnosis of a condition or disease and/or a risk of the user having the condition or disease based on the scale-obtained data. For example, the scale-obtained data of a first user indicates that the user is overweight, recently had an increase in weight, and has a risk of having atrial fibrillation. The first user is identified as a user corresponding with priority user data. A second user of the scale has scale-obtained data indicating a decrease in recovery parameters (e.g., time to return to baseline parameters) and the user inputs an indication that they are training for a marathon. The second user is also identified as a user corresponding with priority user data. The scale displays indications to the user with the priority user data, in some embodiments, on how to use to the scale to communicate the user data to external circuitry for further processing, correlation, and/or other features, such as social network connections. Further, the scale, in response to the priority, displays various feedback to the user, such as user-targeted advertisements and/or suggestions, directly on the FUI and/or another electronic device. In some embodiments, only users with priority user data have data output to the external circuitry to determine risks, although embodiments in accordance with the present disclosure are not so limited

In various embodiments, the scale displays identification of other circuitry that the scale apparatus is configured to communicate with. The other circuitry may be identified by the output circuit and the FUI displays the identification. Responsive to an input from the user's foot selecting one or more of the identified other circuitry (e.g., electronic device), user data is output to the selected other circuitry using the output circuit. The output may be, for example, only in response to verifying identification of the user, such as using a biometric obtained using the scale. In this manner, the user can output data to another device and can control the output to prevent accidental disclosure of personal data.

As the FUI includes a limited amount of space to display data, in various embodiments, the FUI displays portions and/or subsets of data using a color coding to abbreviate more complex information, such as cardiac-information. As an example, a green color indicates a good value, a yellow color indicates an okay value, and a red color indicates a bad value. Such values include physiologic parameters, weight, weight increase or decrease, recovery parameters, among other information. In a specific example, the FUI displays a simple display of “weight” with the color coding corresponding to the user's weight change. If the user has a goal to lose weight and has lost weight, the scale displays “weight” with a green coding. If the user has not lost weight or gained weight, the scale displays “weight” with a yellow coding. If the user gained weight, the scale displays “weight” with a red coding. A user without a weight loss goal (or with a muscle gain goal) may have different color codings. For example, if the user lost weight or has not lost weight, “weight” with a green coding is displayed. If the user gained an amount of weight that is below a threshold, “weight” with a yellow coding is displayed. If the user gained an amount of weight that is above the threshold, “weight” with a red coding is displayed.

The various thresholds, settings, color coding or goals can be set by the user and/or determined by the scale based on user input data. Further, multiple different data types with color codings are displayed simultaneously and/or sequentially, such as weight, body-mass-index, heart rate, heart age, PWV, PTT, among other parameters. The scale communicates the information with other electronic devices, including the color coding and additional information about the values and color coding. In a number of embodiments, the additional information is displayed on the other device using the same color coding so that the data corresponds between the scale and other devices. In accordance with a number of embodiments, the scale performs a question and answer session. For example, the FUI can display a plurality of questions using the user display. Using user interaction by the user's foot, the FUI receives user inputs (e.g., answers) to each of the questions and, using the output circuit, stores the user inputs within a user profile associated with the user. For example, the FUI provides a number of questions in a question and answer session to identify symptoms, health or fitness goals, categories of interest, demographic information, and/or other data from the user. As previously described, the scale can (alternatively and/or in addition to a FUI or GUI) have a voice input/output circuitry that can obtain user's answers to questions via voice comments and inputs user information in response (e.g., a speaker component to capture voice sounds from the user and processing circuitry to recognize the voice commands and/or speech).

In various embodiments, the scale and/or other external circuitry further processes the user data. For example, the scale and/or external circuitry or the scale determines additional health data such as at least one physiologic parameter using the user data. Example physiologic parameters includes PWV, BCG, respiration, arterial stiffness, cardiac output, pre-ejection period, stroke volume, and a combination thereof. The external circuitry then outputs the physiologic parameter.

In various embodiments, the scale and/or other external circuitry determines additional physiological parameters, clinical indications, and/or additional health information. Clinical indication data is data indicative of physical state of the user, such as a disease, disorder, and/or risk for a disease or disorder and can be used for assessment of a condition or treatment of the user. The clinical indication data, in various embodiments, includes information that is regulated by a government agency, such as the Food and Drug Administration (FDA), and/or otherwise requires a prescription from a physician for the user to obtain. Example clinical indication data includes physiological parameters, risk factors, and/or other indicators that the user has a condition or could use a treatment. For example, the user can be correlated with the condition or treatment by comparing the cardio-related data to reference information. The reference information can include a range of values of the cardio-related data for other users having the corresponding condition or treatment indicators. The other user are of a demographic background of the user, such that the reference information includes statistical data of a sample census. For example, the clinical indication data can be derived, that is indicative of a physiologic status of the user, and for assessment of a condition or treatment of the user and using the cardio-related data and/or historical cardio-related data within a user profile corresponding with the user. The condition or treatment can correspond to the physiologic status.

The additional health information, includes derived measurements and/or generic health information that may be “non-regulated” by agencies, such as the FDA. In various embodiments, the additional health information can be indicative of the clinical indication data and/or can correlate to categories of interest provided by the user. For example, the additional health information can include non-prescription health information such as generic health information including disease or disorder symptoms, risk, or advice, generic health information related to categories of interest provided by the user, and/or generic health information that correlated to the cardio-related data. In some embodiments, the additional health information is based on historical data. For example, the additional health information (e.g., a table) provided can include a correlation to the categories of interest and the user data over time. The categories of interest, in number of embodiments, include demographics of interest, symptoms of interest, disorders of interest, diseases of interest, drugs of interest, treatments of interest, etc.

As previously discussed, in accordance with a number of embodiments, the scale using the FUI provides a number of questions to the user. In various embodiments, the questions include asking if the user is interested in additional health information and if the user has particular categories of interest. In various embodiments, the categories of interest include a set of demographics, disorders, diseases, and/or symptom, drugs, treatments that the user is interested, and/or other topics. In some embodiments, the scale provides the user input to external circuitry and the external circuitry derives additional health information for the user. The additional health information can include a table that corresponds to the categories of interest and/or corresponds to the physiological parameter and/or clinical indications determined without providing any specific values and/or indication related to the physiological parameter. The user is provided the additional health information by the external circuitry outputting the information to the scale and/or another user-device, and the scale and/or other user-device displays the information. In various embodiments, the information is printed by the user. In various related-aspects, the scale using the processing circuitry 104 generates the additional health information instead of the external circuitry.

For example, in some instances, a user may not realize they are having a symptom and may not input the symptom unless directly ask. As a specific example, the user may be having shortness of breath when exercising or difficulty sleeping. The user may not identity that the shortness of breath or difficulty sleeping is a symptom for a condition or may forget that they are experiencing such a symptom. In response to identifying the user may have condition that is associated with the symptom of shortness of breath or difficulty sleeping, the scale asks the user if the user is experiencing such a symptom (without directly identifying this as a symptom) and can store the response for use by a physician or to verify the clinical indication data.

The additional health information is generated, in various embodiments, by comparing and/or correlating the categories of interest to raw data obtained by the data-procurement circuitry or the user data to historical user data captured over a period of time. In various embodiments, the correlation/comparison include comparing statistical data of a sample census pertinent to the categories of interest and the at least one physiological parameter. The statistical data of a sample census includes data of other users that are correlated to the categories of interest. In such instances, the additional health information includes a comparison of data measured while the user is standing on the platform to sample census data. In other related embodiments, the correlation/comparison includes comparing statistical data of a sample census pertinent to the categories of interest and values of the least one physiological parameter of the sample census. In such instances, the additional health information includes average physiological parameter values of the sample census that is set by the user, via the categories of interest, and may not include actual values corresponding to the user.

For example, if the categories of interest are demographic categories, the additional health information can include various physiological parameter values of average users in the demographic categories and/or values of average users with a clinical indication that correlates to a physiological parameter of the user. Alternatively and/or in addition, the additional health information can include general medical insights related to the categories of interest. For example, “Did you know if you are over the age of 55 and have gained 15 pounds, you are at risk for a particular disease/disorder?” The scale can ask the user if the user would like to include this factor or disease in their categories of interest to dynamically update the categories of interest of the user. The physician of the user can be notified of the user's interest and/or can be provided a copy of the additional health information. For example, the physician may go over the additional health information with the user during an appointment to provide further clarity.

Various categories of interest, in accordance with the present disclosure, include demographics of the user, disorders, diseases, symptoms, prescription or non-prescription drugs, treatments, past medical history, family medical history, genetics, life style (e.g., exercise habits, eating habits, work environment), among other categories and combinations thereof. In a number of embodiments, various physiological factors can be an indicator for a disease and/or disorder. For example, an increase in weight, along with other factors, can indicate an increased risk of atrial fibrillation. Further, atrial fibrillation is more common in men. However, symptoms of various disorders or disease can be different depending on categories of interest (e.g., atrial fibrillation symptoms can be different between men and women). For example, in women, systolic blood pressure is associated with atrial fibrillation. In other instances, sleep apnea may be assessed via an ECG and can be correlated to weight of the user. Furthermore, various cardiac conditions can be assessed using an ECG. For example, atrial fibrillation can be characterized and/or identified in response to a user having indistinguishable or fibrillating p-waves, and indistinguishable baseline/inconsistent beat fluctuations. Atrial flutter, by contrast, can be characterized by having indistinguishable p-wave, variable heart rate, having QRS complexes, and a generally regular rhythm. Ventricular tachycardia (VT) can be characterized by a rate of greater than 120 beats per minute, and short or broad QRS complexes (depending on the type of VT). Atrio-Ventricular (AV) block can be characterized by PR intervals that are greater than normal (e.g., a normal range for an adult is generally 0.12 to 0.20 seconds), normal-waves, QRS complexes can be normal or prolong shaped, and the pulse can be regular (but slow at 20-40 beats per minute). For more specific and general information regarding atrial fibrillation and sleep apnea, reference is made herein to https://www.clevelandclinicmeded.com/medicalpubs/diseasemanagement/cardiology/atrial-fibrillation/ and http://circ.ahajournals.org/content/118/10/1080.full, which are fully incorporated herein for their specific and general teachings. Further, other data and demographics that are known and/or are developed can be added and used to derive additional health information.

For example, the categories of interest for a particular user can include a change in weight, age 45-55, and female. The scale obtains raw data using the data-procurement circuitry 138 and the categories of interest from the user using the FUI and/or a GUI of another user device in communication with the scale. The scale outputs the raw data and categories of interest to the external circuitry and the external circuitry correlates the categories of interest to the raw data and derives additional health information therefrom. Further, the external circuitry, over time, historically collects and correlates the categories of interest of the user and data from the data-procurement circuitry using the specific patient profile of the user. The external circuitry, in various embodiments, sends the data to a physician and/or additional health information to the user (to print and/or otherwise view).

In various embodiments, the scale is used by multiple different users. One or more of the different users can have other peripheral devices (e.g., user devices) with GUI's and/or the specific user may have different preferences of where to display data between the FUI and the GUI. For example, a first user may not have other devices and/or prefers to view data while standing on the scale. The first user may be older than a second user. The second user has another user device and often views data on the GUI of the user device. A third user may be taller and older than both the first and second user, and may have user device. When the first user stands on the scale, the scale recognizes the first user and displays data to the first user via the FUI and at font and/or size that is larger than when the second user stands on the scale. When the second user stands on the scale, the scale recognizes the second user and some data (e.g., default data such as weight) and an indication of that other data can be viewed on the user device is displayed on the FUI and the second user can view the other data via the GUI of the user device. Similarly, when the third user stands on the scale, the scale recognizes the third user and display some default data and/or indication of available additional data via the FUI of the scale (and at a size and/or font that is larger than what is displayed for the first users). The third user can then view the additional data via the GUI.

The scale can be used in different settings and can have different display defaults depending on the different settings. The different settings can include a consumer setting, a professional setting, and/or a combination. A consumer setting can include use of the scale in a location of a consumer, such that the multiple users known one another. A professional setting can include use of the scale in the location of a professional and/or a business, such as a medical office, an exercise facility, a nursing home, etc. In a professional setting, the different users may not know one another and/or know each other less closely than in a consumer setting. A combination can include use of the scale in a location of the consumer with data being output to a professional and/or use of the scale in a location of a professional or business with data output to the user. Data provided to the user and/or the professional can default to be displayed on the FUI of the scale, the GUI of the user device, and/or a GUI of other external circuitry depending on the use of the scale. Depending on the setting, the scale defaults to different default displays. In a consumer setting and/or combination setting, data can default to display on the FUI of the scale. The defaulted display of data can be revised by the user providing inputs to display the data on the GUI of the user device or a GUI of another external circuitry (e.g., a standalone CPU) and/or automatically by the scale based on past scale-based actions of the user. As a specific example, a first user provided a user input to the scale to display data on the GUI of the user device multiple times (e.g., more than a threshold number of times, such as five times). In response, the scale adjusts the defaulted display and output data to the GUI of the user device. In a professional setting, the scale is not owned by the user. The user may be uninterested in synchronizing their user device with the professional's scale. The display may default to the GUI of the user device to display an option to synchronize. Alternatively, the display may default to the FUI of the scale to display an option to synchronize and, responsive to user verification or authority to synchronize, defaults to display on the GUI of the user device. During the combination consumer/professional setting, portions of scale-obtained data for a particular user may default to display on external circuitry, such as a standalone or server CPU that is accessible by the professional. The scale, in various embodiments, is aware of the setting based on inputs to the scale, a default setting, and/or querying users of the scale.

FIG. 1C shows an isometric view of a multifunction scale 100 with a large-area display (beneath platform 115), consistent with various aspects of the present disclosure. In this particular embodiment, the scale 100 has a primarily rectangular shape with a support structure 110 around the perimeter of the scale that transfers the weight of a user on the platform 115 through load cells in each corner of the support structure 110. In other implementations, the scale has rounded corners, is elliptical or shaped to be circular or circularly elongated. In embodiments in which the shape of the scale does not indicate the intended orientation of the user's feet (e.g., circular or circularly elongated), the scale recognizes (as described herein) where the feet are located and orients the information to be displayed accordingly as though the intended orientation of the user's feet is however/wherever the user's feet are oriented on the scale.

It is to be understood that the aesthetic design of the scale 100 may take on a plurality of shapes and sizes (based on the needs of the users, e.g., weight requirements, their aesthetic preferences, etc.). A feature of the multifunction scale 110 is the large-area display that makes up the majority of the top surface of the scale. The display may present the user with a myriad of information, such as the results of physiological and biometric test results conducted by the scale, entertainment information (while the scale is conducting tests or a weight measurement), and aesthetic screen savers.

As illustrated by FIG. 1C, the user interact with the scale using a FUI displayed on the user display 115. For example, the user can provide various inputs using the FUI using their foot. In some embodiments, the user selects an icon or item from a list to cause the scale to perform a particular features, measurement, and/or test. The FUI, in response to various measurements, displays a user weight, other cardio-related data, and/or other health data and/or non-health data. Further, the FUI displays a variety of questions and the user can answer using foot-based user inputs. Such questions can be used to identity symptoms, goals, and/or other information from the user. The user, in some embodiments, provides inputs via their foot contacting a virtual keyboard display using the FUI. Furthermore, the FUI can display other circuitry that the scale can communicate with. In response to user input selecting one of the other circuitry (in response to verification of the user's identity using a biometric obtained using the scale), data is output from the scale to the other circuitry and/or from the other circuitry to the scale.

FIG. 1D shows an isometric, cross-sectional view of a multifunction scale 100 with a support structure 110 that integrates a large-area display 120, a platform 115, and circuitry 130 (including at least user-targeted circuitry, and a communication driver), consistent with various aspects of the present disclosure. The platform 115 above the display 120 transmits the weight of a user to the support structure 110 and away from the display, while also transmitting touch-capacitive signals indicative of a user's position and/or movement on the platform 115, through the display, to the scale circuitry 130. The support structure 110 is attached to a bezel which supports the display 120. The support structure 110 further houses load cells equally spaced along the perimeter of the scale 100. Each load cell outputs an electrical signal indicative of a mass transmitted from the user through the load cell to the scale circuitry (which interprets the electrical signals and presents the weight of the user on the display). A plurality of translucent electrode leads are embedded into the platform 115 to provide electrical signals to the scale circuitry 130, and the electrical signals are interpreted by the scale circuitry 130 as being indicative of a condition of a user, with the condition being presented on the display 120 for the user.

In an embodiment in accordance with FIG. 1D, a scale 100 includes a support structure 110 having a platform 115 with sensor circuitry therein (e.g., electrodes). The platform 115 engages a user with the sensor circuitry while the user stands on the platform 115 and use the sensor circuitry to collect physiological data from the user. While the user stands on the scale 100, the display 120 displays data through and throughout the entire platform. The scale 100 also includes circuitry 130 including processing circuitry, data-procurement circuitry, and an output circuit. The processing circuitry (e.g., user-targeted circuitry) receives the physiological data from the data-procurement circuitry and determines physiological parameters of the user, including a user-weight metric, while the user stands on and engages the force sensor circuitry of the platform 115. The output circuit provides information from the processing circuitry to the display 120 for viewing by the user through the platform 115. In some embodiments the circuitry 130 may also include a memory circuit to store user-specific data including stored physiological parameters of the user in response to or developed by the processing circuitry and to store physiological parameters of the user determined by the processing circuitry (in other embodiments, the memory circuit may be external to the scale 100, and may be accessed by the circuitry 130 over a communication network).

Load bearing characteristics of the multifunction scale 100 may provide both functionality and longevity. The platform 115, in conjunction with the support structure 110 (and the bezel), minimizes the load transfer to the display 120 while still maintaining sufficient conductivity through the platform 115 (e.g., a glass platform or other clear material) to the display 120 to allow for touch-screen functionality. If the platform 115 is too compliant, under the user's weight, excessive force exerted on the display 120 may cause damage. If the platform 115 is not conductively coupled to the display 120 (e.g., due to a gap there-between), touch-screen functionality of the scale 100 may be challenging. Accordingly, FIG. 1D discloses one embodiment that addresses such issues via a platform 115 that transfer weight to the support structure 110 with minimal compliance, by which the display 120 remains conductively coupled to the platform 115 while preventing excessive force from being exerted on the display 120 (that would otherwise cause damage).

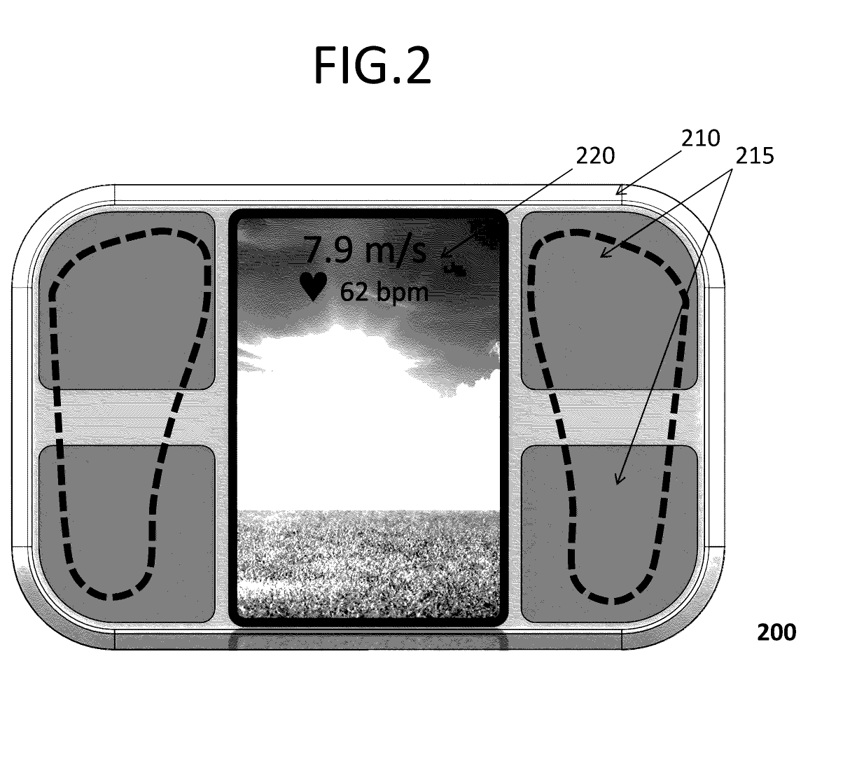

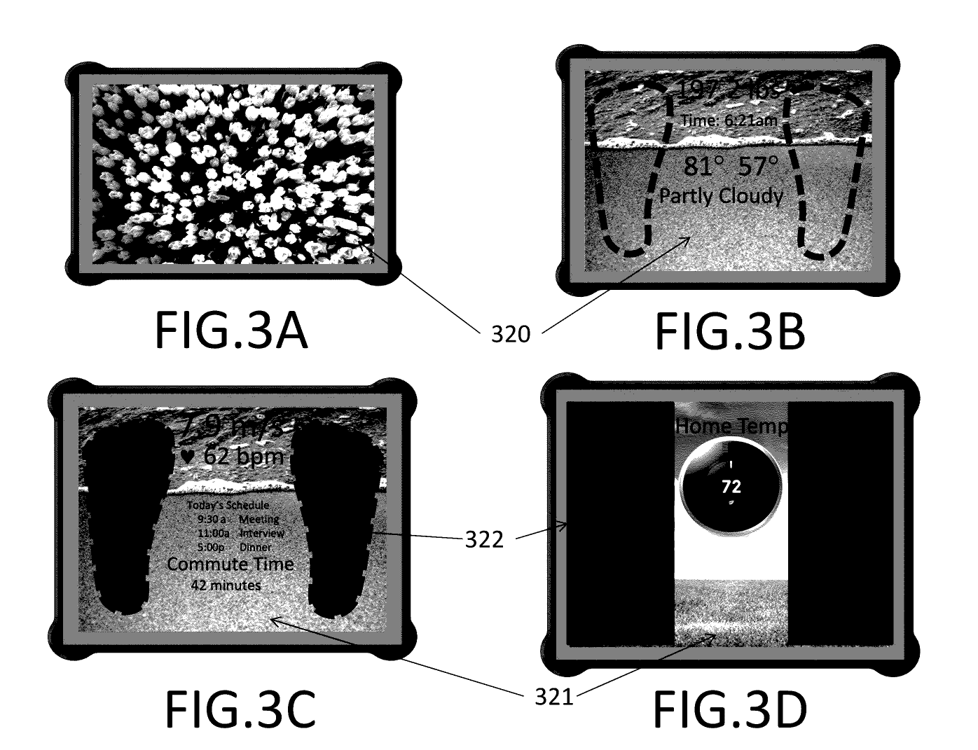

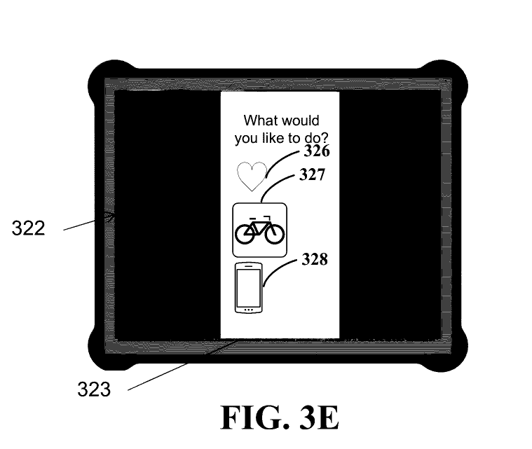

In certain specific embodiments of the present disclosure, as shown in FIG. 2, multifunction scale 200 includes a support structure 210 which integrates a large-area display 220 and a platform 210 (where the user will stand when the scale 200 is in use). In such an embodiment, the display 220 is essentially the full length of the scale 20, but not full width. This display size is closer in dimensions to a tablet computing device (such as an iPad). The force sensor circuitry in the platform 210 includes electrodes for physiological and biometric sensing. As discussed in more detail below in reference to FIGS. 3A-D, the display 220 is capable of presenting a myriad of information to the user.

FIG. 3A-E shows top views of a number of multifunction scale displays, consistent with various aspects of the present disclosure. FIG. 3A presents an exemplary image that may be selected by a user as a “screen saver,” and displayed by the scale, in a large-area display mode 320, when not in use. In further embodiments, the scale, when not in use may enter “sleep mode” and present or display pleasant still or video images, including a slide-show of images selected by the user, such as family-photos or other pleasant preferred images or animations. In more specific embodiments of the present disclosure, a camera is communicatively coupled to the multifunction scale and operates with facial recognition software for identifying the user and greeting the user (“Hello Michael”). The apparatus may also identify the user based on multiple biometrics/measurement characteristics (e.g., weight, body composition, body mass index (BMW), body fat percentage, PWV, etc., alone or correlated with additional measurements), and greet the user accordingly. Based on the identified user, the scale may operate in accordance with user-specific aspects as may relate to physiology or preferences such as for a “screen saver.” For instance, biometric and physiological tests can be conducted, with the test results saved to the identified user's file (and/or the results sent to a user's doctor for further review and analysis), as well as a number of other functionalities, such as playing the user's favorite musical artist and loading the display to present the user with pertinent information. Further, the scale may offer multiple modes that the user may choose between to ensure greater accuracy of physiological testing results, such as “athlete mode” for users that are very active.

As shown in FIG. 3B, a relaxing ambience may be provided to the room where the scale is located, such as by displaying a video of waves lapping over sand, in a large-area display mode of the display 320 (when the user is not standing on the scale). In some embodiments, the scale plays an audio track associated with the video. In FIGS. 3C-D, a reduced-area display mode 321 is utilized when the user is standing on the scale. In such an embodiment, the display area where the user is standing, 322 is turned-off as the user's feet prevent the user from seeing this portion of the screen 322, and the disabling of the display area 322 reduces battery consumption of the display 320.

In FIGS. 3B-D, while the scale conducts tests on the user (e.g., weight measurements, body fat, biometric and physiological tests (e.g., ballistocardiogram (BCG) or pulse wave velocity (PWV), etc.) or whenever the user desires, the user is able to access other information from the scale such as the user's current weight, pulse rate, and time of day, among other user-configurable information. In further more specific embodiments (as shown in FIGS. 3B-D), the scale displays general or user-specific information, such as weather conditions, stocks, news, traffic conditions, home climate (e.g., screening air quality, oxygen level, temperature), commute times, user's daily schedule, personal reminders, or other information as may be collected by the scale via a wired or wireless connection to the internet, or to a smart device (e.g., a hand-held mobile or cellular phone, smart watch or other wearable electronic device, or tablet) or to fixed computing device (e.g., as a phone or watch) using the foot-based GUI. Information displayed on the scale is shown in an appropriately scaled font, composition and orientation to be readable from a standing position. As shown in FIG. 3D, in implementations of the disclosure directed to smart-homes, a multifunction scale user controls (via the touch-screen display) a plurality of other devices throughout the home such as a climate control system, security system, operation of the shower, etc. The electronic communications between the multifunction scale and the various devices may take the form of either wireless or wired communications.

The user display can display a FUI, which as shown in FIG. 3E, can include a variety of icons that represent functions that the scale can perform. The functions can include a physiological test, a parameter to output, data to output, authorization for communication, authorization or activation of services, among other actions. As a specific example, the heart-shaped icon 326 can be indicative of a feature for comparing and/or analyzing trends heart rate and/or other cardiac parameters (e.g., BCG, PWV), the bike icon 327 may be indicative of feature or service including a fitness test, and the cellphone icon 328 may be indicative of authorization to pair with or otherwise output data to an identified external circuitry. Although the embodiment of 3E illustrates three icons, embodiments are not so limited and can include greater or less than three icons. In response to the user touching one of the icons with their foot, such as with their toe, the FUI is revised to verify the selection. For example, the FUI displays text asking “Did you want to perform test A?” and include two icons listing “yes” or “no”. In response to the user selecting yes, the scale performs the test or other function. In response to the user selecting no, the scale redisplays the variety of icons for the user to re-select on the FUI.

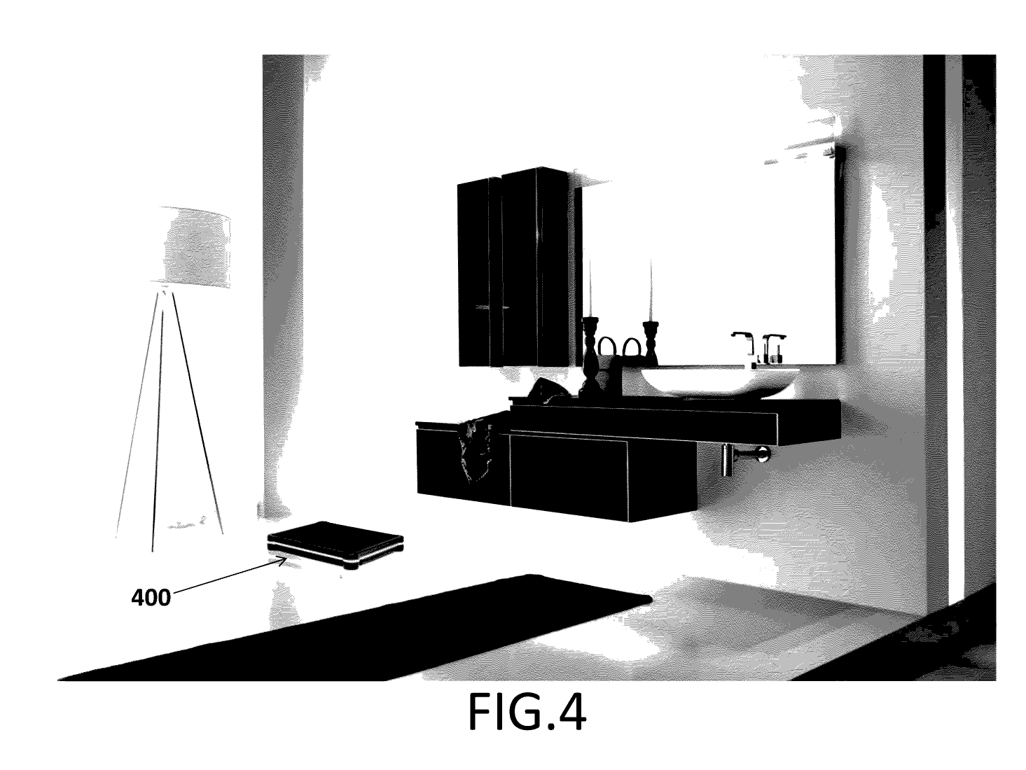

FIG. 4 shows a multifunction scale 400 with large-area display (e.g., for a bathroom), consistent with various aspects of the present disclosure. In the present embodiment, the multifunction scale 400 includes circuitry, such as a camera and image processing circuitry. The camera may be directed at the floor below the scale or the surrounding area. Based on the images processed (by the image processing circuitry) of the area surrounding the scale, the multifunction scale's large-area display depicts an image that mimics the surrounding area when idle. As shown in FIG. 4, the room is primarily furnished in black and white. The image processing circuitry identifies this black and white room theme based on the images captured by the camera and selects a color or combination of colors in a pattern or design that would mimic the décor of the room. As a result, the scale 400 is more likely to blend into the décor of the room and minimize the likelihood that the scale 400 will detract from the ambiance. In embodiments where the camera is directed at the floor, the scale 400 depicts an image indicative of the flooring below the scale 400, which would similarly minimize any detraction of aesthetics the scale would otherwise cast through its visually non-conforming presence. In either embodiment discussed above, when the multifunction scale 400 is idle, from a glance the scale 400 is effectively camouflaged. In other embodiments, the user and/or an interior designer may select a theme for the display based on the desired look for the room where the multifunction scale 400 is placed.

FIG. 5A is a flowchart depicting an example manner in which a user specific physiologic meter or scale may be programmed in accordance with the present disclosure. This flowchart uses a computer processor circuit (or CPU) along with a memory circuit shown herein as user profile memory 546A. The CPU operates in a low-power consumption mode, which may be in off mode or a low-power sleep mode, and at least one other higher power consumption mode of operation. In more specific embodiments, the low-power sleep mode and/or night-operative mode reduces consumption of power while using all or a portion of the display to provide a low-level night light (which can also be activated by motion recognition of the user whereby otherwise the display is off or dark during night-time periods).

As exemplary circuits for transitioning between such a low-power and higher power modes, the central processing unit (CPU) can be integrated with presence and/or motion sense circuits, such as a passive infrared (PIR) circuit and/or pyroelectric PIR circuit. In a typical application, the PIR circuit provides a constant flow of data indicative of amounts of radiation sensed in a field of view directed by the PIR circuit. For instance, the PIR circuit can be installed behind an upper surface which is transparent to infrared light (and/or other visible light) of the platform and installed at an angle so that the motion of the user approaching the platform apparatus is sensed. Radiation from the user, upon reaching a certain detectable level, wakes up the CPU which then transitions from the low-power mode, as depicted in block 540, to a regular mode or active mode of operation. In alternative embodiments, the CPU transitions from the low-power mode of operation in response to another remote/wireless input used as an intrusion to awaken the CPU. In other embodiments, user motion can be detected by an accelerometer integrated in the scale or the motion is sensed with a single integrated microphone or microphone array, to detect the sounds of a user approaching.

Accordingly, from block 540, flow proceeds to block 542 where the user or other presence is sensed as data is received at the scale. At block 544, the circuitry assesses whether the received data qualifies as requiring a wake up. If not, flow turns to block 540. If however, wake up is required, flow proceeds from block 544 to block 546 where the CPU assesses whether a possible previous user has approached the platform apparatus. This assessment is performed by the CPU accessing the user profile memory 546A and comparing data stored therein for one or more such previous users with criteria corresponding to the received data that caused the wake up. Such criteria includes, for example, the time of the day (early morning or late morning), the pace at which the user approached the platform apparatus as sensed by the motion detection circuitry, the height of the user as indicated by the motion sensing circuitry and/or a camera installed and integrated with the CPU, and/or more sophisticated bio-metric data provided by the user and/or automatically by the circuitry in the platform apparatus.

As discussed herein, such sophisticated circuitry can include one or more of the following user-specific attributes: foot length, type of foot arch, weight of user, and/or manner and speed at which the user steps onto the platform apparatus, or sounds made by the user's motion or by user speech (e.g., voice). In some embodiments, facial or body-feature recognition may also be used in connection with the camera and comparisons of images therefrom to images in the user profile memory.

From block 546, flow proceeds to block 548 where the CPU obtains and/or updates user corresponding data in the user profile memory. As a learning program is developed in the user profile memory, each access and use of the platform apparatus is used to expand on the data and profile for each such user. From block 548, flow proceeds to block 550 where a decision is made regarding whether the set of electrodes at the upper surface of the platform is ready for the user, which may be based on the data obtained from the user profile memory. For example, delays may ensue from the user moving his or her feet about the platform, as may occur while certain data is being retrieved by the CPU (whether internally or from an external source such as a program or from the Internet cloud) or when the user has stepped over a certain area configured for providing display information back to the user. If the electrodes are not ready for the user, flow proceeds from block 550 to block 552 to accommodate this delay.

Once the CPU determines that the electrodes are ready for use while the user is standing on the platform, flow proceeds to block 560. Stabilization of the user on the platform may be ascertained by injecting current through the electrodes via the interleaved arrangement thereof. Where such current is returned via other electrodes for a particular foot and/or foot size, and is consistent for a relatively brief period of time (e.g., a few seconds), the CPU can assume that the user is standing still and ready to use the electrodes and related circuitry.

At block 560, a decision is made that both the user and the scale are ready for measuring impedance and certain segments of the user's body, including at least one foot.

The remaining flow of FIG. 5A includes the application and sensing of current through the electrodes for finding the optimal electrodes (562) and for performing impedance measurements (block 564). These measurements are continued until completed at block 566 and useful measurements are recorded and are logged in the user profile for this specific user, at block 568. At block 572, the CPU generates output data to provide feedback as to the completion of the measurements and, as maybe indicated as a request via the user profile for this user, as an overall report on the progress for the user relative to previous measurements made for this user and that are stored in the user profile memory. Such feedback may be shown on the display via the foot-based GUI, through a speaker with co-located apertures in the platform's housing for audible reception by the user, and/or by vibration circuitry which, upon vibration under control of the CPU, the user can sense through one or both feet while standing on the scale. From this output at block 572, flow returns to the low-power mode as indicated at block 574 with the return to the beginning of the flow at block 540.

FIG. 5B shows current paths 500 through the body of a user 505 standing on a scale 510 for the IPG trigger pulse and Foot IPG, consistent with various aspects of the present disclosure. Impedance measurements 515 are measured when the user 505 is standing and coverings over the feet such as socks or shoes, within the practical limitations of capacitive-based impedance sensing, with energy limits considered safe for human use. The measurements 515 can also be made with non-clothing material placed between the user's feet and contact electrodes, such as thin films or sheets of plastic, glass, paper or wax paper, where the electrodes operate within energy limits considered safe for human use. The IPG measurements also can be sensed in the presence of callouses on the user's feet that normally diminish the quality of the signal. As shown in FIG. 5B, the user 505 is standing on a scale 510, where the tissues of the user's body will be modeled as a series of impedance elements, and where the time-varying impedance elements change in response to cardiovascular and non-cardiovascular movements of the user. ECG and IPG measurements are sensed through the feet and can be challenging to take due to small impedance signals with (1) low SNR, and because they are (2) frequently masked or distorted by other electrical activity in the body such as the muscle firings in the legs to maintain balance. The human body is unsteady while standing still, and constant changes in weight distribution occur to maintain balance. As such, cardiovascular signals that are measured with weighing scale-based sensors typically yield signals with poor SNR, such as the Foot IPG and standing BCG. Thus, such scale-based signals require a stable and high quality synchronous timing reference, to segment individual heartbeat-related signals for signal averaging to yield an averaged signal with higher SNR versus respective individual measurements.

The ECG can be used as the reference (or trigger) signal to segment a series of heartbeat-related signals measured by secondary sensors (optical, electrical, magnetic, pressure, microwave, piezo-based, etc.) for averaging a series of heartbeat-related signals together, to improve the SNR of the secondary measurement. The ECG has an intrinsically high SNR when measured with body-worn gel electrodes, or via dry electrodes on handgrip sensors. In contrast, the ECG has a low SNR when measured using foot electrodes while standing on said scale platforms; unless the user is standing perfectly still to eliminate electrical noise from the leg muscles firing due to body motion. As such, ECG measurements at the feet while standing are considered to be an unreliable trigger signal (low SNR). Therefore, it is often difficult to obtain a reliable cardiovascular trigger reference timing when using ECG sensors incorporated in base scale platform devices. Both Inan, et al. (IEEE Transactions on Information Technology in Biomedicine, 14:5, 1188-1196, 2010) and Shin, et al. (Physiological Measurement, 30, 679-693, 2009) have shown that the ECG component of the electrical signal measured between the two feet while standing was rapidly overpowered by the electromyogram (EMG) signal resulting from the leg muscle activity involved in maintaining balance.

The accuracy of cardiovascular information obtained from weighing scale platforms is also influenced by measurement time. The number of beats obtained from heartbeat-related signals for signal averaging is a function of measurement time and heart rate. The Mayo Clinic cites that typical resting heart rates range from 60 to 100 beats per minute. Therefore, short signal acquisition periods may yield a low number of beats to average, which may cause measurement uncertainty, also known as the standard error in the mean (SEM). SEM is the standard deviation of the sample mean estimate of a population mean. Where, SE is the standard error in the samples N, which is related to the standard error or the population S. The following is an example SE for uncorrelated noise:

$$S E = \frac{S}{\sqrt{N}}$$

For example, a five second signal acquisition period may yield a maximum of five to eight beats for ensemble averaging, while a 10 second signal acquisition could yield 10-16 beats. However, the number of beats available for averaging and SNR determination is usually reduced for the following factors; (1) truncation of the first and last ensemble beat in the recording by the algorithm, (2) triggering beats falsely missed by triggering algorithm, (3) cardiorespiratory variability, (4) excessive body motion corrupting the trigger and Foot IPG signal, and (5) loss of foot contact with the measurement electrodes.

Sources of noise can require multiple solutions for SNR improvements for the signal being averaged. Longer measurement times increase the number of beats lost to truncation, false missed triggering, and excessive motion. Longer measurement times also reduce variability from cardiorespiratory effects. If shorter measurement times (e.g., less than 30 seconds) are desired for scale-based sensor platforms, sensing improvements need to tolerate body motion and loss of foot contact with the measurement electrodes.

The human cardiovascular system includes a heart with four chambers, separated by valves that return blood to the heart from the venous system into the right side of the heart, through the pulmonary circulation to oxygenate the blood, which then returns to the left side of the heart, where the oxygenated blood is pressurized by the left ventricles and is pumped into the arterial circulation, where blood is distributed to the organs and tissues to supply oxygen. The cardiovascular or circulatory system is designed to ensure oxygen availability and is often the limiting factor for cell survival. The heart normally pumps five to six liters of blood every minute during rest and maximum cardiac output during exercise increases up to seven-fold, by modulating heart rate and stroke volume. The factors that affect heart rate include autonomic innervation, fitness level, age and hormones. Factors affecting stroke volume include heart size, fitness level, contractility or pre-ejection period, ejection duration, preload or end-diastolic volume, afterload or systemic resistance. The cardiovascular system is constantly adapting to maintain a homeostasis (set point) that minimizes the work done by the heart to maintain cardiac output. As such, blood pressure is continually adjusting to minimize work demands during rest. Cardiovascular disease encompasses a variety of abnormalities in (or that affect) the cardiovascular system that degrade the efficiency of the system, which include but are not limited to chronically elevated blood pressure, elevated cholesterol levels, edema, endothelial dysfunction, arrhythmias, arterial stiffening, atherosclerosis, vascular wall thickening, stenosis, coronary artery disease, heart attack, stroke, renal dysfunction, enlarged heart, heart failure, diabetes, obesity and pulmonary disorders.

Each cardiac cycle results in a pulse of blood being delivered into the arterial tree. The heart completes cycles of atrial systole, delivering blood to the ventricles, followed by ventricular systole delivering blood into the lungs and the systemic arterial circulation, where the diastole cycle begins. In early diastole the ventricles relax and fill with blood, then in mid-diastole the atria and ventricles are relaxed and the ventricles continue to fill with blood. In late diastole, the sinoatrial node (the heart's pacemaker) depolarizes then contracting the atria, the ventricles are filled with more blood and the depolarization then reaches the atrioventricular node and enters the ventricular side beginning the systole phase. The ventricles contract and the blood is pumped from the ventricles to arteries.

The ECG is the measurement of the heart's electrical activity and is described in five phases. The P-wave represents atrial depolarization, the PR interval is the time between the P-wave and the start of the QRS complex. The QRS wave complex represents ventricular depolarization. The QRS complex is the strongest wave in the ECG and is frequently used as a timing reference for the cardiovascular cycle. Atrial repolarization is masked by the QRS complex. The ST interval represents the period of zero potential between ventricular depolarization and repolarization. The cycle concludes with the T-wave representing ventricular repolarization.

The blood ejected into the arteries creates vascular movements due to the blood's momentum. The blood mass ejected by the heart first travels headward in the ascending aorta and travels around the aortic arch then travels down the descending aorta. The diameter of the aorta increases during the systole phase due to the high compliance (low stiffness) of the aortic wall. Blood traveling in the descending aorta bifurcates in the iliac branch which transitions into a stiffer arterial region due to the muscular artery composition of the leg arteries. The blood pulsation continues down the leg and foot. Along the way, the arteries branch into arteries of smaller diameter until reaching the capillary beds where the pulsatile blood flow turns into steady blood flow, delivering oxygen to the tissues. The blood returns to the venous system terminating in the vena cava, where blood returns to the right atrium of the heart for the subsequent cardiac cycle.

Surprisingly, high quality simultaneous recordings of the Leg IPG and Foot IPG are attainable in a practical manner (e.g., a user operating the device correctly simply by standing on the impedance body scale foot electrodes), and can be used to obtain reliable trigger fiducial timings from the Leg IPG signal. This acquisition can be far less sensitive to motion-induced noise from the Leg EMG that often compromises Leg ECG measurements. Furthermore, it has been discovered that interleaving the two Kelvin electrode pairs for a single foot, result in a design that is insensitive to foot placement within the boundaries of the overall electrode area. As such, the user is not constrained to comply with accurate foot placement on conventional single foot Kelvin arrangements, which are highly prone to introducing motion artifacts into the IPG signal, or result in a loss of contact if the foot is slightly misaligned. Interleaved designs begin when one or more electrode surfaces cross over a single imaginary boundary line separating an excitation and sensing electrode pair. The interleaving is configured to maintain uniform foot surface contact area on the excitation and sensing electrode pair, regardless of the positioning of the foot over the combined area of the electrode pair.

Various aspects of the present disclosure include a weighing scale (e.g., scale 110) of an area sufficient for an adult of average size to stand comfortably still and minimize postural swaying. The nominal scale length (same orientation as foot length) is 12 inches and the width is 12 inches. The width can be increased to be consistent with the feet at shoulder width or slightly broader (e.g., 14 to 18 inches, respectively).

FIG. 6A shows an example of the insensitivity to foot placement 600 on scale electrode pairs 605/610 with multiple excitation paths 620 and sensing current paths 615, consistent with various aspects of the present disclosure. An aspect of the platform is that it has a thickness and strength to support a human adult of at least 200 pounds without fracturing, and another aspect of the device platform is comprised of at least six electrodes, where the first electrode pair 605 is solid and the second electrode pair 610 is interleaved. Another aspect is that the first and second interleaved electrode pairs 605/610 are separated by a distance of at least 40+/−5 millimeters, where the nominal separation of less than 40 millimeters has been shown to degrade the single Foot IPG signal. Another key aspect is the electrode patterns are made from materials with low resistivity such as stainless steel, aluminum, hardened gold, ITO, index matched ITO (IMITO), carbon printed electrodes, conductive tapes, silver-impregnated carbon printed electrodes, conductive adhesives, and similar materials with resistivity lower than 300 ohms/sq. In the certain embodiments, the resistivity is below 150 ohms/sq. Other embodiments include the electrodes being built into a display material (e.g., glass or plastics) and being made from substantially translucent materials that blend in with the display material such as doped/implanted patterns (e.g., by ionic doping or implanting or layering with ions and/or nanostructures) in order to provide the electrodes in the form of conductive lines in or on (under) the display material. The electrodes are connected to the electronic circuitry in the scale by routing the electrodes around the edges of the scale to the surface below, or through at least one hole in the scale (e.g., a via hole).

Suitable electrode arrangements for dual Foot IPG measurements can be realized in other embodiments. In certain embodiments, the interleaved electrodes are patterned on the reverse side of a thin piece (e.g., less than 2 mm) of high-ion-exchange (HIE) glass, which is attached to a scale substrate and used in capacitive sensing mode. In certain embodiments, the interleaved electrodes are patterned onto a thin piece of paper or plastic which can be rolled up or folded for easy storage. In certain embodiments, the interleaved electrodes are integrated onto the surface of a tablet computer for portable IPG measurements. In certain embodiments, the interleaved electrodes are patterned onto a kapton substrate that is used as a flex circuit.

In certain embodiments, the scale area has a length of 10 inches with a width of eight inches for a miniature scale platform. Alternatively, the scale may be larger (up to 36 inches wide) for use in bariatric class scales.

In the present disclosure, the leg and foot impedance measurements can be simultaneously carried out using a multi-frequency approach, in which the leg and foot impedances are excited by currents modulated at two or more different frequencies, and the resulting voltages are selectively measured using a synchronous demodulator. This homodyning approach can be used to separate signals (in this case, the voltage drop due to the imposed current) with very high accuracy and selectivity.

This measurement configuration is based on a four-point configuration in order to minimize the impact of the contact resistance between the electrode and the foot, a practice well-known in the art of impedance measurement. In this configuration the current is injected from a set of two electrodes (the “injection” and “return” electrodes), and the voltage drop resulting from the passage of this current through the resistance is sensed by two separate electrodes (the “sense” electrodes), usually located in the path of the current. Since the sense electrodes are not carrying any current (by virtue of their connection to a high-impedance differential amplifier), the contact impedance does not significantly alter the sensed voltage.

In order to sense two distinct segments of the body (the legs and the foot), two separate current paths are defined by electrode positioning. Therefore two injection electrodes are used, each connected to a current source modulated at a different frequency. The injection electrode for leg impedance is located under the plantar region of the left foot, while the injection electrode for the Foot IPG is located under the heel of the right foot. Both current sources share the same return electrode located under the plantar region of the right foot. This is an illustrative example. Other configurations may be used.

The sensing electrodes can be localized so as to sense the corresponding segments. Leg IPG sensing electrodes are located under the heels of each foot, while the two foot sensing electrodes are located under the heel and plantar areas of the right foot. The inter-digitated nature of the right foot electrodes ensures a four-point contact for proper impedance measurement, irrespectively of the foot position, as already explained.

FIG. 6B shows an example of electrode configurations, consistent with various aspects of the disclosure. As shown by the electrode connections, in some embodiments, ground is coupled to the heel of one foot of the user (e.g., the right foot) and the foot current injection (e.g., excitation paths 220) is coupled to the toes of the respective one foot (e.g., toes of the right foot). The leg current injection is coupled to the toes of the other foot (e.g., toes of the left foot).

FIG. 6C shows an example of electrode configurations, consistent with various aspects of the disclosure. As shown by the electrode connections, in some embodiments, ground is coupled to the heel of one foot of the user (e.g., the right foot) and the foot current injection (e.g., excitation paths 220) is coupled to the toes of the one foot (e.g., toes of the right foot). The leg current injection is coupled to the heels of the other foot of the user (e.g., heels of the left foot).

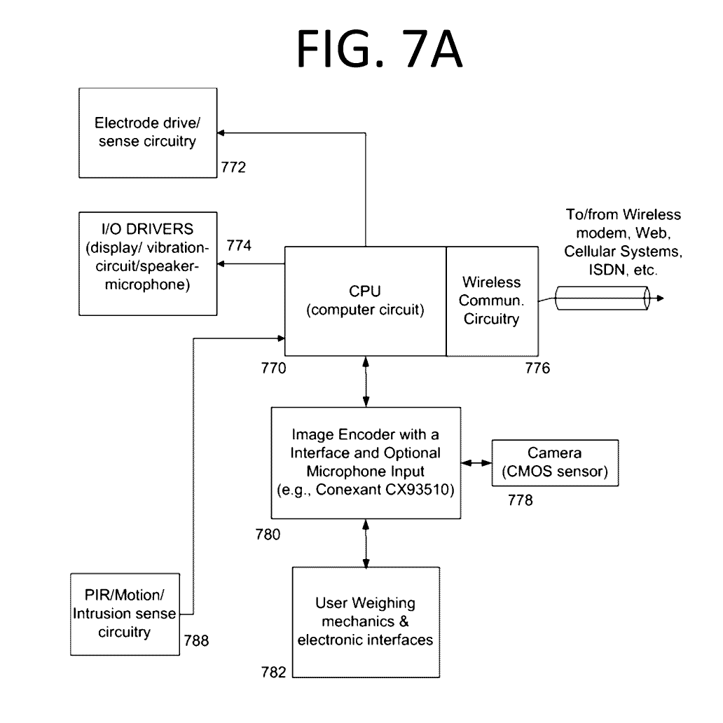

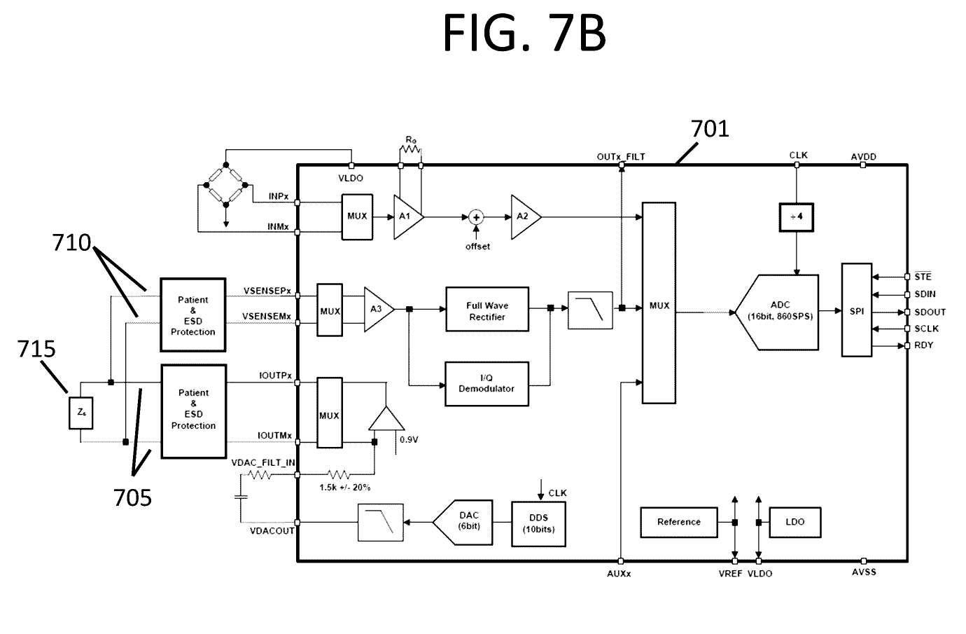

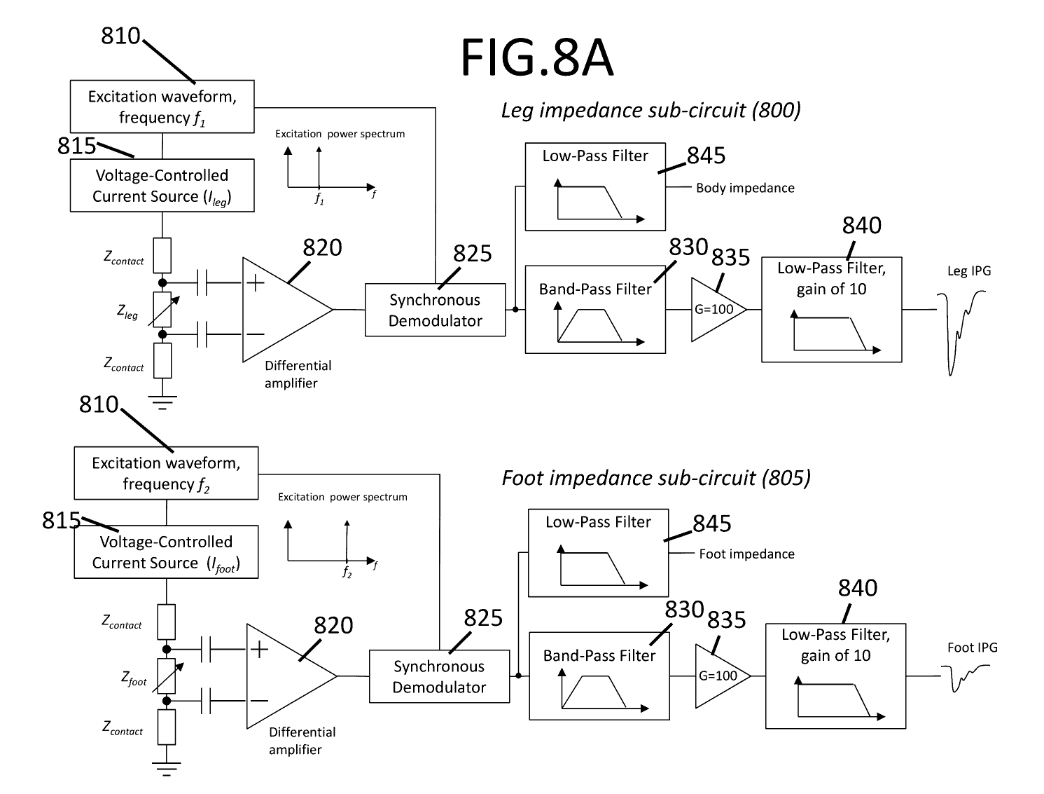

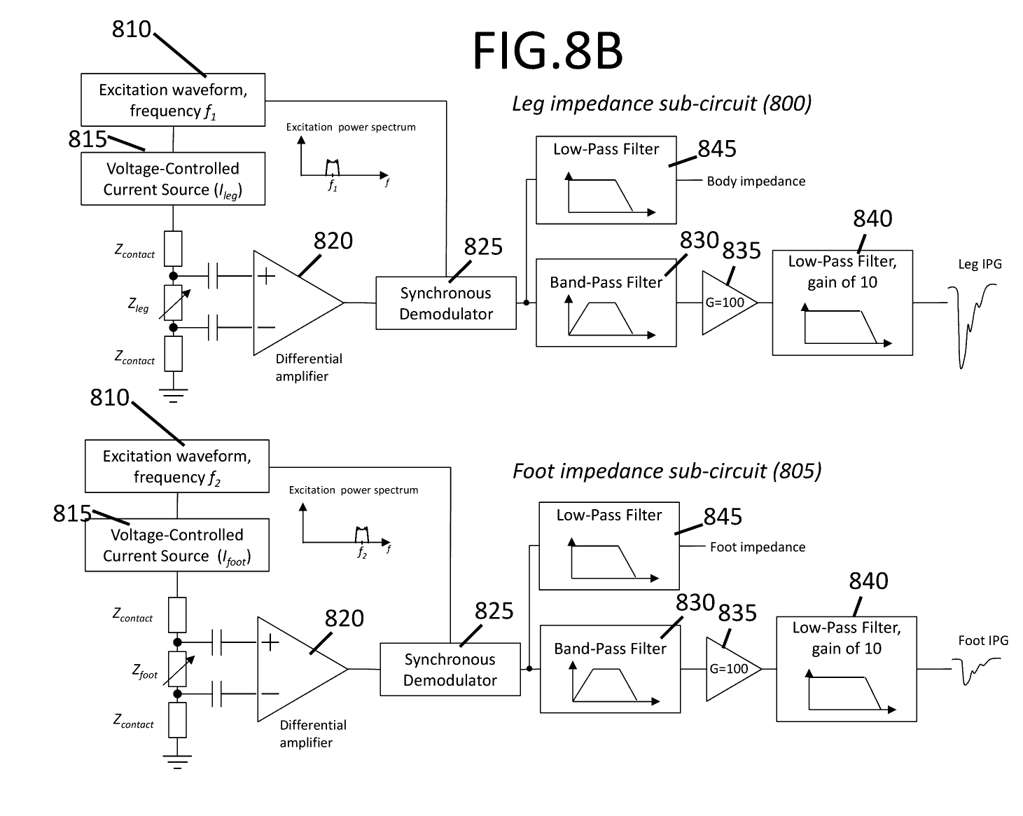

FIG. 7A depicts an example block diagram of circuitry for operating core circuits and modules of the multifunction scale, used in various specific embodiments of the present disclosure. Consistent with yet further embodiments of the present disclosure, FIG. 7A depicts an example block diagram of circuitry for operating core circuits and modules, including, for example, the operation of a CPU with the related and more specific circuit blocks/modules in FIGS. 8A-8B. As shown in the center of FIG. 7A, the main computer circuit 770 is shown with other previously-mentioned circuitry in a generalized manner without showing some of the detailed circuitry such as for amplification and current injection/sensing (772). The computer circuit 770 can be used as a control circuit with an internal memory circuit for causing, processing and/or receiving sensed input signals as at block 772. As discussed, these sensed signals can be responsive to injection current and/or these signals can be sensed at least for initially locating positions of the foot or feet on the platform area, by less complex grid-based sense circuitry surrounding the platform area as is conventional in capacitive touch-screen surfaces which, in certain embodiments, the platform area includes.

As noted, the memory circuit can be used not only for the user profile memory, but also to provide configuration and/or program code and/or other data such as user-specific data from another authorized source such as from a user monitoring his/her logged data and/or profile from a remote desk-top. The remote device or desktop can communicate with and access such data via a wireless communication circuit 776 via a wireless modem, router, ISDN channel, cellular systems, Bluetooth and/or other broadband pathway or private channel. For example, the wireless communication circuit 776 can provide an interface between an app on the user's cellular telephone/tablet (e.g., phablet, IPhone and/or IPad) and the platform apparatus, wherefrom the IPhone can be the output/input interface for the platform (scale) apparatus including, for example, an output display, speaker and/or microphone, and vibration circuitry.

A camera 778 and image encoder circuit 780 (with compression and related features) can be incorporated as an option. As discussed above, the scale, as in block 782, also optionally includes housing which encloses and/or surrounds the platform.

For long-lasting battery life in the scale (batteries not shown), at least the CPU 770, the wireless communication circuit 776, and other current draining circuits are inactive unless and until activated in response to the intrusion/sense circuitry 788. As shown, one specific implementation employs a Conexant chip (e.g., CX93510) to assist in the low-power operation. This type of circuitry is designed for motion sensors configured with a camera for visual verification and image and video monitoring applications (such as by supporting JPEG and MJPEG image compression and processing for both color and black and white images). When combined with an external CMOS sensor, the chip retrieves and stores compressed JPEG and audio data in an on-chip memory circuit (e.g., 256 KB/128 KB frame buffer) to alleviate the necessity of external memory. The chip uses a simple register set via the microprocessor interface and allows for wide flexibility in terms of compatible operation with another microprocessor.

In one specific embodiment, a method of using the platform with the plurality of electrodes concurrently contacting a limb of the user, includes operating such to automatically obtain measurement signals from the plurality of electrodes. As noted above, these measurement signals might initially be through less-complex (e.g., capacitive grid-type) sense circuitry. Before or while obtaining a plurality of measurement signals, the signal-sense circuitry 788 is used to sense wireless-signals indicative of the user approaching the platform and, in response, cause the CPU circuitry 770 to transition from a reduced power-consumption mode of operation and at least one higher power-consumption mode of operation. After the circuitry is operating in the higher power-consumption mode of operation, the CPU accesses the user-corresponding data stored in the memory circuit and thereafter causes a plurality of impedance-measurement signals to be obtained using the plurality of electrodes; therefrom, the CPU generates signals corresponding to cardiovascular timings of the user, and such physiological measurements are communicated via the display.

This method can employ the signal-sense circuit as a passive infrared detector and with the CPU programmed (as a separate module) to evaluate whether radiation from the passive infrared detector is indicative of a human. For example, sensed levels of radiation that would correspond to a live being that has a size which is less than a person of a three-foot height, and/or not being sensed as moving for more than a couple seconds, can be assessed as being a non-human.

Accordingly, should the user be recognized as human, the CPU is activated and begins to attempt the discernment process of which user might be approaching. This is performed by the CPU accessing the user-corresponding data stored in the memory circuit (the user profile memory). The user can be recognized based on parameters such as discussed above (e.g., time of morning, speed of approach, etc.) and/or from physiologic parameters stored in the memory circuit and attributable to the user's previously-measured physiologic parameters. For further information regarding such user-recognition (and/or circuitry-power saving approaches), reference may be made to U.S. patent documents: application Ser. No. 14/338,266, entitled Device And Method Having Automatic User-Responsive and User-Specific Physiological-Meter Platform (PHYW.005CIP1), filed Jul. 22, 2014; Application Ser. No. 14/498,773, entitled Fitness Testing Scale (PHYW.029PA), filed Sep. 26, 2014; and to Provisional Application No. 62/027,724, entitled Multi-Function Scale With Large Area Display (PHYW.015P1), filed Jul. 22, 2014, each being incorporated by reference in their entirety as well as for the aspects specifically noted herein.

The CPU can also select one of a plurality of different types of user-discernible visual/audible/tactile information and for presenting the discerned user with visual/audible/tactile information that was retrieved from the memory as being specific to the user. For example, user-selected visual/audible data can be outputted for the user. Also, responsive to the motion detection indication, the camera can be activated to capture at least one image of the user while the user is approaching the platform (and/or while the user is on the platform to log confirmation of the same user with the measured impedance information). As shown in block 774 of FIG. 7A, where a speaker is also integrated with the CPU, the user can simply command the scale to start the process and activation proceeds.

In another such method, the circuitry of FIG. 7A is used with the plurality of electrodes being interleaved and engaging the user, as a combination weighing scale (via block 782) and a physiologic user-specific impedance-measurement device. By using the impedance-measurement signals and obtaining at least two impedance-measurement signals between one foot of the user and another location of the user, the interleaved electrodes assist the CPU in providing measurement results that indicate one or more of the following user-specific attributes as being indicative or common to the user: foot impedance, foot length, and type of arch, and wherein one or more of the user-specific attributes are accessed, by being read or stored, in the memory circuit and identified as being specific to the user. This information can be retrieved by the user, medical and/or security personnel, according to a data-access authorization protocol as might be established upon initial configuration for the user.

FIG. 7B shows an exemplary block diagram depicting the circuitry for interpreting signals received from electrodes. The input electrodes 705 transmit various electrical signals through the patient's body (depending on the desired biometric and physiological test to be conducted) and output electrodes 710 receive the modified signal as affected by a user's electrical impedance 715. Once received by the output electrodes 710, the modified signal is processed by processor circuitry 701 based on the selected test. Signal processing conducted by the processor circuitry 701 is discussed in more detail below (with regard to FIGS. 8A-B). In certain embodiments of the present disclosure, the circuitry within 701 is provided by Texas Instruments part # AFE4300.

FIGS. 8A-8B show example block diagrams depicting the circuitry for sensing and measuring the cardiovascular time-varying IPG raw signals and steps to obtain a filtered IPG waveform, consistent with various aspects of the present disclosure. The example block diagrams shown in FIGS. 8A-8B are separated into a leg impedance sub-circuit 800 and a foot impedance sub-circuit 805.

Excitation is provided by way of an excitation waveform circuit 310. The excitation waveform circuit 310 provides a stable amplitude excitation signal by way of various wave shapes of various, frequencies, such as more specifically, a sine wave signal (as is shown in FIG. 3a) or, more specifically, a square wave signal (as shown in FIG. 3b). This excitation waveform (of sine, square, or other wave shape) is fed to a voltage-controlled current source circuit 315 which scales the signal to the desired current amplitude. The generated current is passed through a decoupling capacitor (for safety) to the excitation electrode, and returned to ground through the return electrode (grounded-load configuration). Amplitudes of 1 and 4 mA peak-to-peak are typically used for Leg and Foot IPGs, respectively.

The voltage drop across the segment of interest (legs or foot) is sensed using an instrumentation differential amplifier (e.g., Analog Devices AD8421) 320. The sense electrodes on the scale are AC-coupled to the inputs of the differential amplifier 320 (configured for unity gain), and any residual DC offset is removed with a DC restoration circuit (as exemplified in Burr-Brown App Note Application Bulletin, SBOA003, 1991, or Burr-Brown/Texas Instruments INA118 datasheet). Alternatively, a fully differential input amplification stage can be used which eliminates the need for DC restoration.

The signal is then demodulated with a phase-sensitive synchronous demodulator circuit 325. The demodulation is achieved in this example by multiplying the signal by 1 or −1 synchronously in-phase with the current excitation. Such alternating gain is provided by an operational amplifier (op amp) and an analog switch (SPST), such as an ADG442 from Analog Devices). More specifically, the signal is connected to both positive and negative inputs through 10 kOhm resistors. The output is connected to the negative input with a 10 kOhm resistor as well, and the switch is connected between the ground and the positive input of the op amp. When open, the gain of the stage is unity. When closed (positive input grounded), the stage acts as an inverting amplifier with a gain of −1. Further, fully differential demodulators can alternatively be used which employ pairs of DPST analog switches whose configuration can provide the benefits of balanced signals and cancellation of charge injection artifacts. Alternatively, other demodulators such as analog multipliers or mixers can be used. The in-phase synchronous detection allows the demodulator to be sensitive to only the real, resistive component of the leg or foot impedance, thereby rejecting any imaginary, capacitive components which may arise from parasitic elements associated with the foot to electrode contacts.

Once demodulated, the signal is band-pass filtered (0.4-80 Hz) with a band-pass filter circuit 330 before being amplified with a gain of 100 with a non-inverting amplifier circuit 335 (e.g., using an LT1058 operational amplifier from Linear Technology Inc.). The amplified signal is further amplified by 10 and low-pass filtered (cut-off at 20 Hz) using a low-pass filter circuit 340 such as 2-pole Sallen-Key filter stage with gain. The signal is then ready for digitization and further processing. In certain embodiments, the signal from the demodulator circuit 325 can be passed through an additional low-pass filter circuit 345 to determine body or foot impedance.

In certain embodiments, the generation of the excitation voltage signal, of appropriate frequency and amplitude, is carried out by a microcontroller, such as an MSP430 (Texas Instruments, Inc.) or a PIC18Fxx series (Microchip Technology, Inc.). The voltage waveform can be generated using the on-chip timers and digital input/outputs or pulse width modulation (PWM) peripherals, and scaled down to the appropriate voltage through fixed resistive dividers, active attenuators/amplifiers using on-chip or off-chip operational amplifiers, as well as programmable gain amplifiers or programmable resistors. In certain embodiments, the generation of the excitation frequency signal can be accomplished by an independent quartz crystal oscillator whose output is frequency divided down by a series of toggle flip-flops (such as an ECS-100AC from ECS International, Inc., and a CD4024 from Texas Instruments, Inc.). In certain embodiments, the generation of the wave shape and frequency can be accomplished by a direct digital synthesis (DDS) integrated circuit (such as an AD9838 from Analog Devices, Inc.). In certain embodiments, the generation of the wave shape (either sine or square) and frequency can be accomplished by a voltage-controlled oscillator (VCO) which is controlled by a digital microcontroller, or which is part of a phase-locked loop (PLL) frequency control circuit. Alternatively, the waveforms and frequencies can be directly generated by on- or off-chip digital-to-analog converters (DACs).

In certain embodiments, the shape of the excitation is not square, but sinusoidal. Such configuration would reduce the requirements on bandwidth and slew rate for the current source and instrumentation amplifier. Harmonics, potentially leading to higher electromagnetic interference (EMI), would also be reduced. Such excitation may also reduce electronics noise on the circuit itself. Lastly, the lack of harmonics from sine wave excitation may provide a more flexible selection of frequencies in a multi-frequency impedance system, as excitation waveforms have fewer opportunities to interfere between each other. Due to the concentration of energy in the fundamental frequency, sine wave excitation could also be more power-efficient. In certain embodiments, the shape of the excitation is not square, but trapezoidal. Alternatively, raised cosine pulses (RCPs) could be used as the excitation wave shape, providing an intermediate between sine and square waves. RCPs could provide higher excitation energy content for a given amplitude, but with greatly reduced higher harmonics.

To further reduce potential electromagnetic interference (EMI), other strategies may be used, such as by dithering the square wave signal (i.e., introducing jitter in the edges following a fixed or random pattern) which leads to so-called spread spectrum signals, in which the energy is not localized at one specific frequency (or a set of harmonics), but rather distributed around a frequency (or a set of harmonics). Because of the synchronous demodulation scheme, phase-to-phase variability introduced by spread-spectrum techniques will not affect the impedance measurement. Such a spread-spectrum signal can be generated by, but not limited to, specialized circuits (e.g., Maxim MAX31C80, SiTime SiT9001), or generic microcontrollers (see Application Report SLAA291, Texas Instruments, Inc.). These spread-spectrum techniques can be combined with clock dividers to generate lower frequencies as well.

As may be clear to one skilled in the art, these methods of simultaneous measurement of impedance in the leg and foot can be used for standard Body Impedance Analysis (BIA), aiming at extracting the relative content of total water, free-water, fat mass and other body composition measures. Impedance measurements for BIA are typically done at frequencies ranging from kilohertz up to several megahertz. The multi-frequency synchronous detection measurement methods described above can readily be used for such BIA, provided that low-pass filtering (345, FIGS. 3a and 3b) instead of band-pass filtering (330, FIGS. 3a and 3b) is performed following the demodulation. In certain embodiments, a separate demodulator channel may be driven by the quadrature phase of the excitation signal to allow the imaginary component of the body impedance to be extracted in addition to the real component. A more accurate BIA can be achieved by measuring both the real and imaginary components of the impedance. This multi-frequency technique can be combined with traditional sequential measurements used for BIA, in which the impedance is measured at several frequencies sequentially. These measurements are repeated in several body segments for segmental BIAs, using a switch matrix to drive the current into the desired body segments.

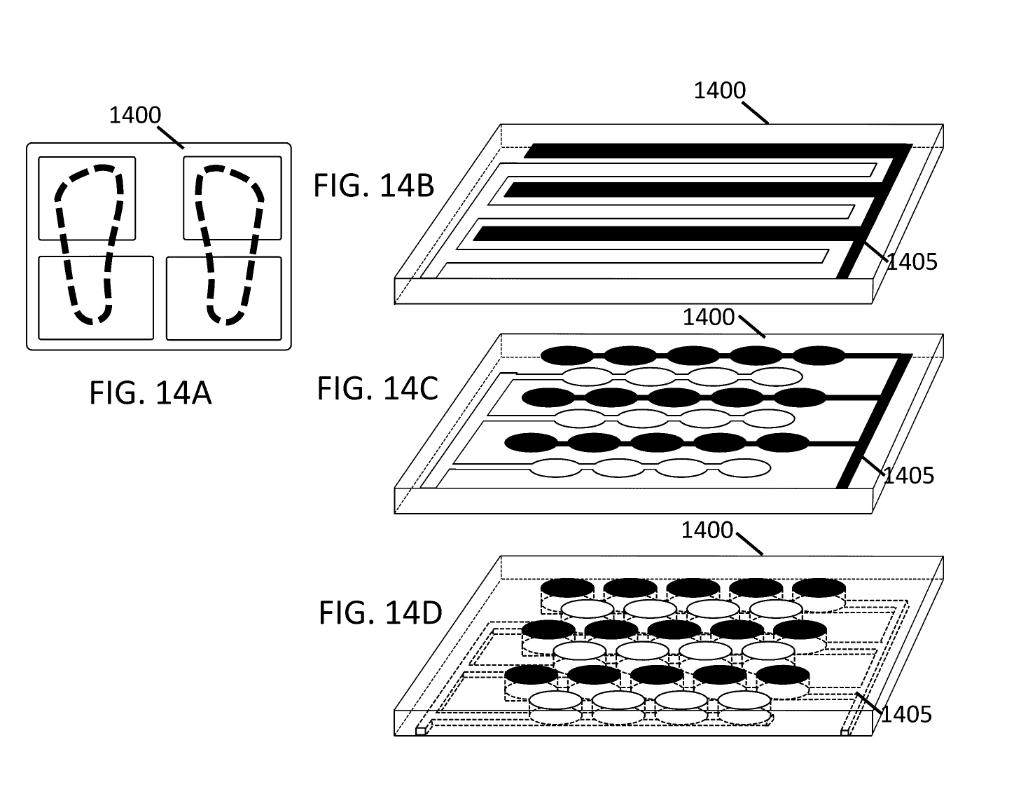

While FIG. 6A shows a circuit and electrode configuration suitable to measure two different segments (legs and one foot), this approach is not readily extendable to more segments due to the shared current return electrode (ground). To overcome this limitation, and provide simultaneous measurements in both feet, the system can be augmented with analog switches to provide time-multiplexing of the impedance measurements in the different segments. This multiplexing can be a one-time sequencing (each segment is measured once), or interleaved at a high-enough frequency that the signal can be simultaneously measured on each segment. The minimum multiplexing rate for proper reconstruction is twice the bandwidth of the measured signal, based on signal processing theory (the Nyquist rate), which equals to about 100 Hz for the impedance signal considered here. The rate must also allow for the signal path to settle in between switching, which usually limits the maximum multiplexing rate. Referring to FIG. 14a, one cycle might start the measurement of the leg impedance and left foot impedances (similarly to previously described, sharing a common return electrode), but then follow with a measurement of the right foot after reconfiguring the switches. For specific information regarding typical switch configurations, reference to U.S. patent application Ser. No. 14/338,266 filed on Oct. 7, 2015, which is fully incorporated for its specific and general teaching of switch configurations.

Since right and left feet are measured sequentially, one should note that a unique current source (at the same frequency) may be used to measure both, providing that the current source is not connected to the two feet simultaneously through the switches, in which case the current would be divided between two paths. One should also note that a fully-sequential measurement, using a single current source (at a single frequency) successively connected to the three different injection electrodes, could be used as well, with the proper switch configuration sequence (no splitting of the current path).

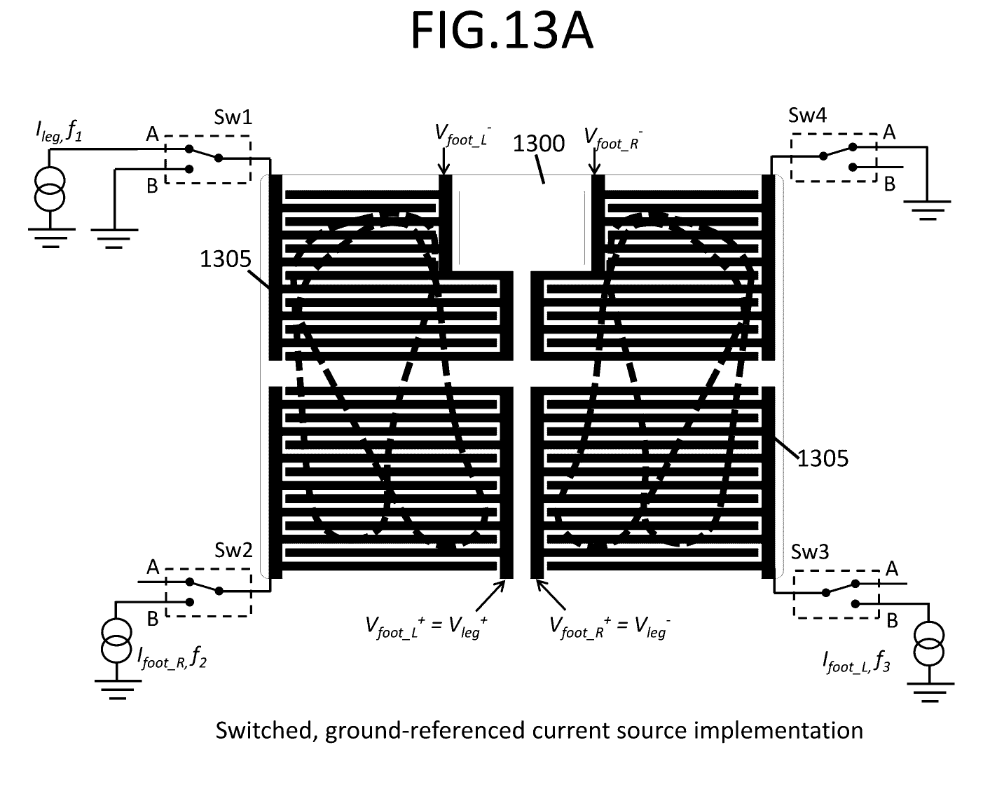

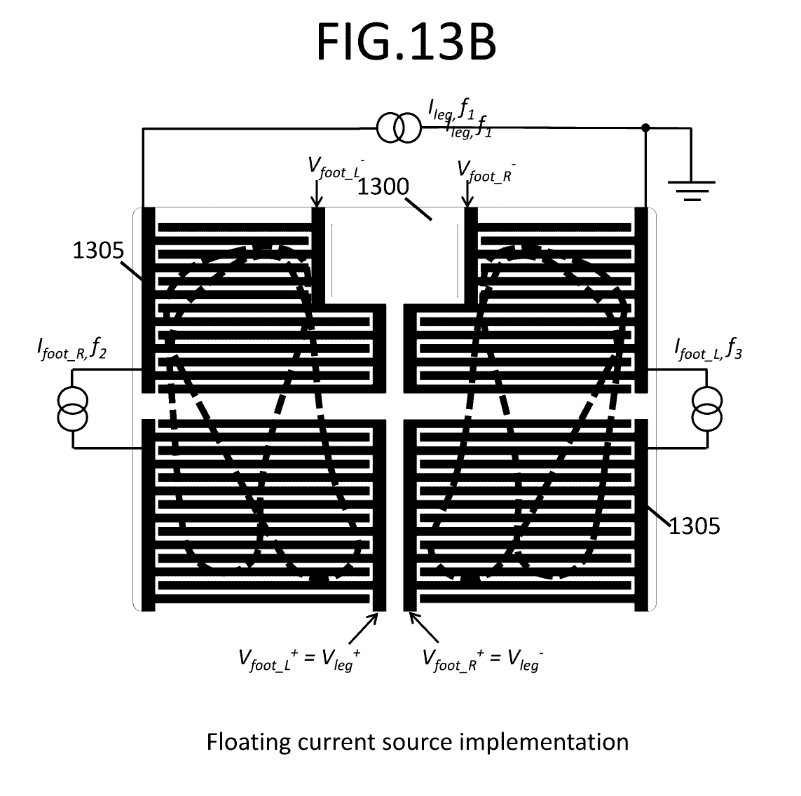

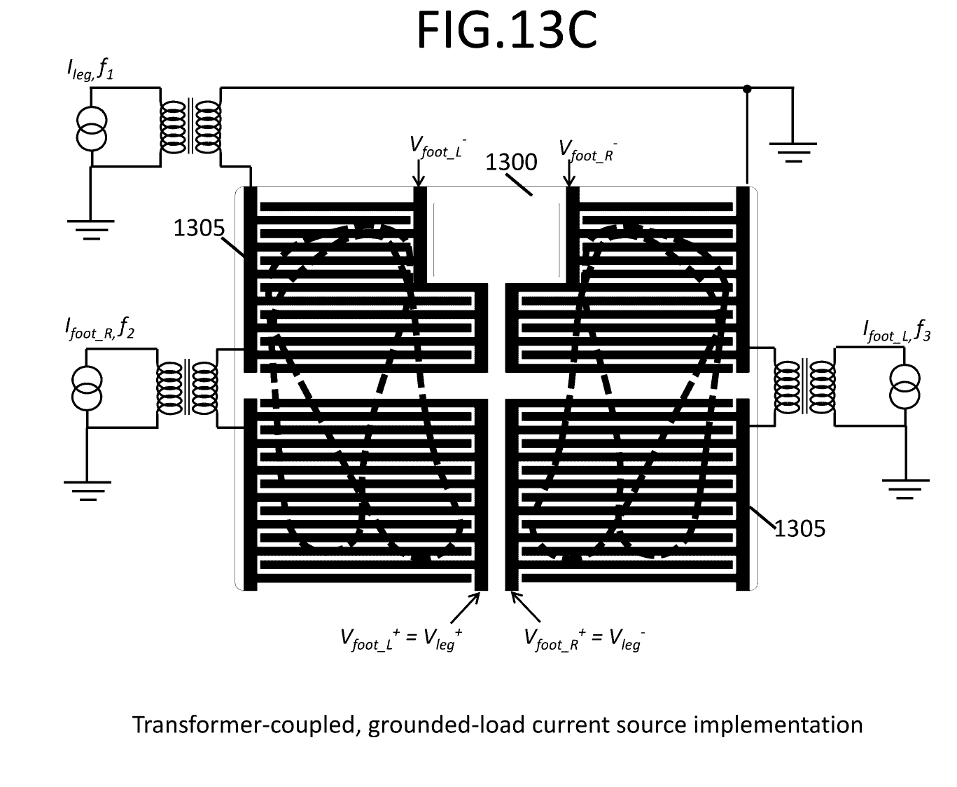

In certain embodiments, the measurement of various body segments, and in particular the legs, right foot and left foot, is achieved simultaneously due to as many floating current sources as segments to be measured, running at separate frequencies so they can individually be demodulated. Such configuration is exemplified in FIG. 13b for three segments (legs, right and left feet). Such configuration has the advantage to provide true simultaneous measurements without the added complexity of time-multiplexing/demultiplexing, and associated switching circuitry. An example of such a floating current source is found in Plickett, et al., Physiological Measurement, 32 (2011). Another approach to floating current sources is the use of transformer-coupled current sources (as depicted in FIG. 14c). Using transformers to inject current into the electrodes enables the use of simpler, grounded-load current sources on the primary, while the electrodes are connected to the secondary. The transformer turns ratio can typically be 1:1, and since frequencies of interest for impedance measurement are typically in the 10-1000 kHz (occasionally 1 kHz for BIA), relatively small pulse transformers can be used. In order to limit the common mode voltage of the body, one of the electrodes in contact with the foot can be grounded.

While certain embodiments presented in the above specification have used current sources for excitation, the excitation can also be performed by a voltage source, where the resulting injection current is monitored by a current sense circuit so that impedance can still be derived by the ratio of the sensed voltage (on the sense electrodes) over the sensed current (injected in the excitation electrodes). It should be noted that broadband spectroscopy methods could also be used for measuring impedances at several frequencies. Combined with time-multiplexing and current switching described above, multi-segment broadband spectroscopy can be achieved.

Various aspects of the present disclosure are directed toward robust timing extraction of the blood pressure pulse in the foot which is achieved by means of a two-step processing. In a first step, the usually high-SNR Leg IPG is used to derive a reference (trigger) timing for each heart pulse. In a second step, a specific timing in the lower-SNR Foot IPG is extracted by detecting its associated feature within a restricted window of time around the timing of the Leg IPG.

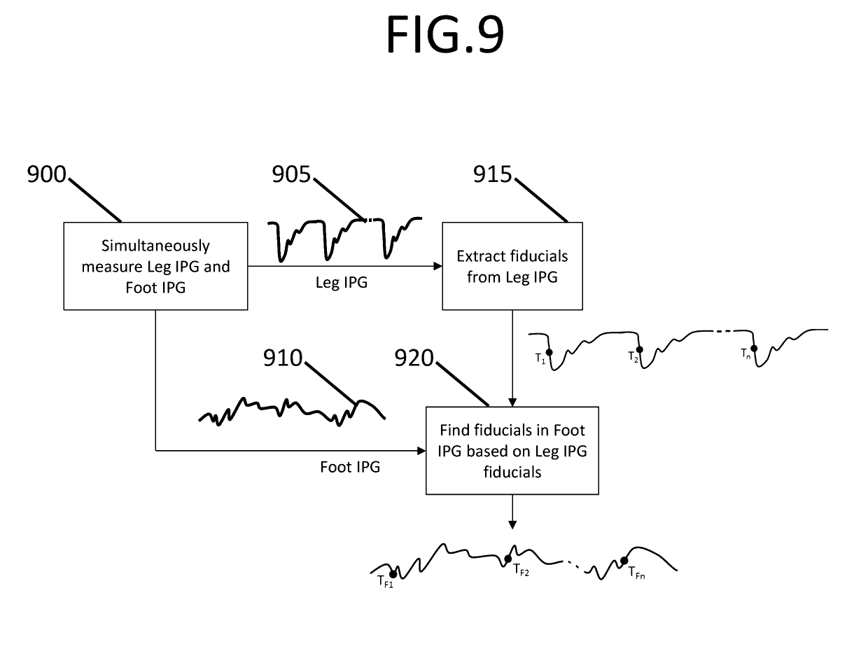

FIG. 9 shows an example block diagram depicting signal processing steps to obtain fiducial references from the individual Leg IPG “beats,” which are subsequently used to obtain fiducials in the Foot IPG, consistent with various aspects of the present disclosure. In the first step, as shown in block 900, the Leg IP and the Foot IPG are simultaneously measured. As shown at 905, the Leg IPG is low-pass filtered at 20 Hz with an 8-pole Butterworth filter, and inverted so that pulses have an upward peak. The location of the pulses is then determined by taking the derivative of this signal, integrating over a 100 ms moving window, zeroing the negative values, removing the large artifacts by zeroing values beyond 15x the median of the signal, zeroing the values below a threshold defined by the mean of the signal, and then searching for local maxima. Local maxima closer than a defined refractory period of 300 ms to the preceding ones are dismissed. The result is a time series of pulse reference timings. At 910, the foot IPG is low-pass filtered at 25 Hz with an 8-pole Butterworth filter and inverted (so that pulses have an upward peak). Segments starting from the timings extracted (block 915) from the Leg IPG (reference timings) and extending to 80% of the previous pulse interval, but no longer than one second, are defined in the Foot IPG. This defines the time windows where the Foot IPG is expected to occur, avoiding misdetection outside of these windows. In each segment, the derivative of the signal is computed, and the point of maximum positive derivative (maximum acceleration) is extracted. The foot of the IPG signal is then computed using an intersecting tangent method, where the fiducial (block 920) is defined by the intersection between a first tangent to the IPG at the point of maximum positive derivative and a second tangent to the minimum of the IPG on the left of the maximum positive derivative within the segment as shown at block 920.

The time series resulting from this two-step extraction is then used in conjunction with another signal to facilitate additional processing. In the present disclosure, these timings are used as reference timings to improve the SNR of BCG signals to subsequently extract intervals between a timing of the BCG (typically the I-wave) and the Foot IPG for the purpose of computing the PWV, as previously disclosed in U.S. 2013/0310700 (Wiard). In certain embodiments, the timings of the Leg IPG are used as reference timings to improve the SNR of BCG signals, and the foot IPG timings are used to extract intervals between timing fiducials of the improved BCG (typically the I-wave) and the Foot IPG for the purpose of computing the PTT and the (PWV).

In certain embodiments, the processing steps include an individual pulse SNR computation after individual timings are extracted, either in Leg IPG or Foot IPG. Following the computation of the SNRs, pulses with a SNR below a threshold value are eliminated from the time series, to prevent propagating noise. The individual SNRs may be computed in a variety of methods known to one skilled in the art. For instance, an estimated pulse can be computed by ensemble averaging segments of signal around the pulse reference timing. The noise associated with each pulse is defined as the difference between the pulse and the estimated pulse. The SNR is the ratio of the root-mean-square (RMS) value of the estimated pulse over the RMS value of the noise for that pulse.

In certain embodiments, the time interval between the Leg IPG pulses, and the Foot IPG pulses, also detected by the above-mentioned methods, is extracted. The Leg IPG measuring a pulse occurring earlier in the legs compared to the pulse from the Foot IPG, the interval between these two is related to the propagation speed in the lower body, i.e., the peripheral vasculature. This provides complementary information to the interval extracted between the BCG and the Foot IPG for instance, and is used to decouple central versus peripheral vascular properties. It is also complementary to information derived from timings between the BCG and the Leg ICG.

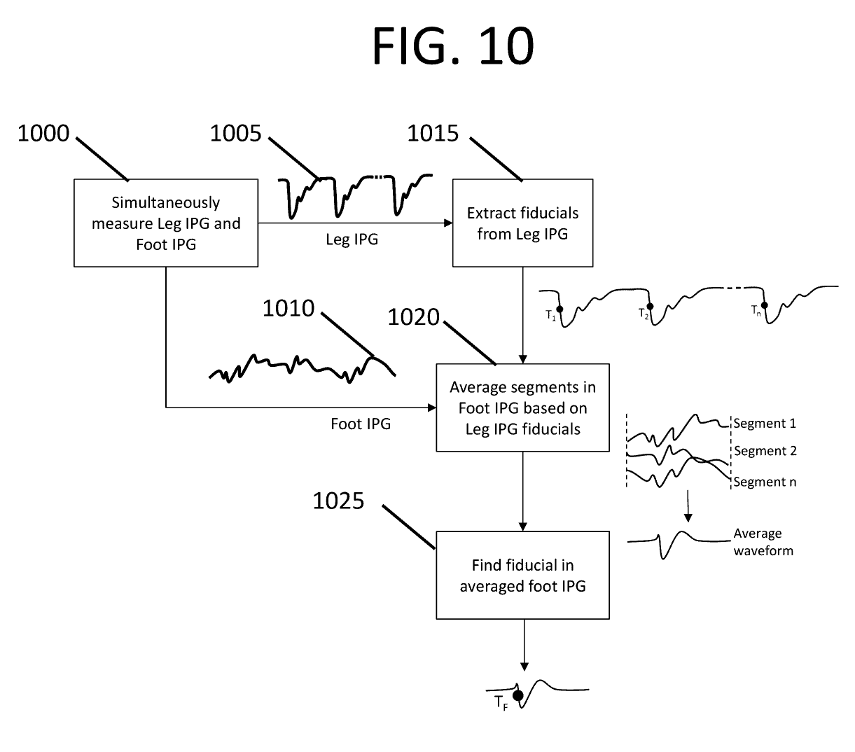

In FIG. 10, the Leg IP and the Foot IPG are simultaneously measured (1000), the Leg IPG is low-pass filtered (block 1005), the foot IPG is low-pass filtered (1010), and segments starting from the timings are extracted (block 1015) from the Leg IPG (reference timings). The segments of the Foot IPG extracted based on the Leg IPG timings are ensemble-averaged (block 1020) to produce a higher SNR Foot IPG pulse. From this ensemble-averaged signal, the start of the pulse is extracted using the same intersecting tangent approach as described earlier. This approach enables the extraction of accurate timings in the Foot IPG even if the impedance signal is dominated by noise. These timings are used together with timings extracted from the BCG for the purpose of computing the PTT and (PWV). Timings derived from ensemble-averaged waveforms and individual waveforms can also be both extracted, for the purpose of comparison, averaging and error-detection.

Specific timings extracted from the IPG pulses (from either leg or foot) are related (but not limited) to the peak of the pulse, the minimum preceding the peak, or the maximum second derivative (maximum rate of acceleration) preceding the point of maximum derivative. An IPG pulse and the extraction of a fiducial (1025) in the IPG can be performed by other signal processing methods, including (but not limited to) template matching, cross-correlation, wavelet-decomposition, or short window Fourier transform.

In certain embodiments, a dual-Foot IPG is measured, allowing the detection of blood pressure pulses in both feet. Such information can be used for diagnostic of peripheral arterial diseases (PAD) by comparing the relative PATs in both feet to look for asymmetries. It can also increase the robustness of the measurement by allowing one foot to have poor contact with electrodes (or no contact at all). SNR measurements can be used to assess the quality of the signal in each foot, and to select the best one for downstream analysis. Timings extracted from each foot can be compared and set to flag potentially inaccurate PWV measurements due to arterial peripheral disease, in the event these timings are different by more than a threshold. Alternatively, timings from both feet are pooled to increase the overall SNR if their difference is below the threshold.

In certain embodiments, the disclosure is used to measure a PWV, where the IPG is augmented by the addition of BCG sensing into the weighing scale to determine characteristic fiducials between the BCG and Leg IPG trigger, or the BCG and Foot IPG. The BCG sensors are comprised typically of the same strain gage set used to determine the bodyweight of the user. The load cells are typically wired into a bridge configuration to create a sensitive resistance change with small displacements due to the ejection of the blood into the aorta, where the circulatory or cardiovascular force produce movements within the body on the nominal order of 1-3 Newtons. BCG forces can be greater than or less than the nominal range in cases such as high or low cardiac output.

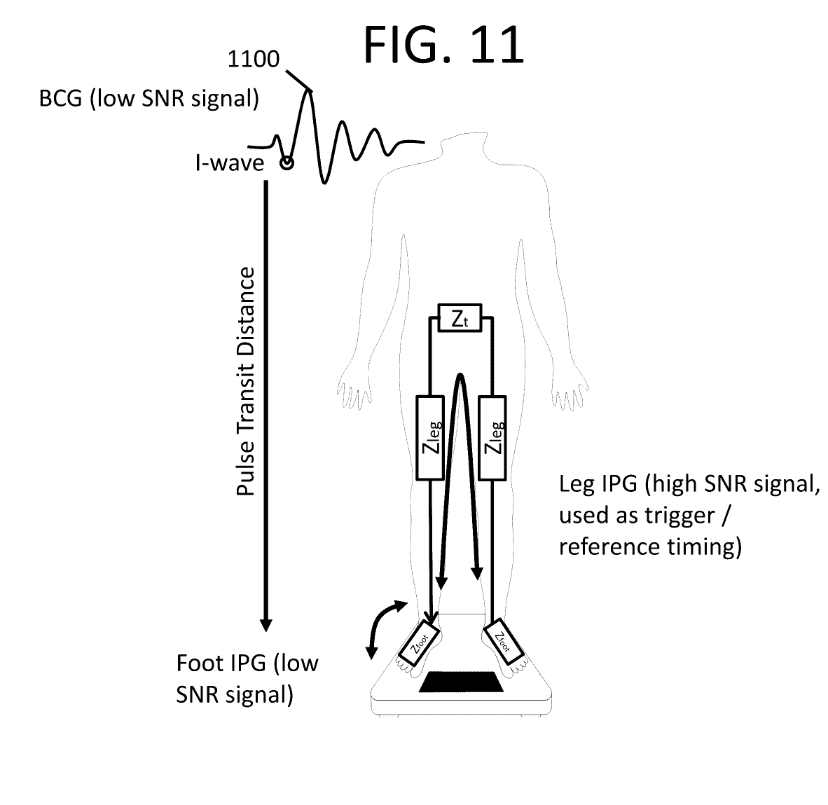

FIG. 11 shows an example configuration to obtain the PTT, using the first IPG as the triggering pulse for the Foot IPG and BCG, consistent with various aspects of the present disclosure. The I-wave of the BCG 1100, as illustrated, normally depicts the headward force due to cardiac ejection of blood into the ascending aorta which can be used as a timing fiducial indicative of the pressure pulse initiation concerning the user's proximal aorta relative to the user's heart. The J-wave is also indicative of timings in the systole phase and also incorporates information related to the strength of cardiac ejection and the ejection duration. The K-Wave also provides systolic and vascular information of the user's aorta. The characteristic timings of these and other BCG waves can be used as fiducials that can be related to fiducials of the IPG signals of the present disclosure.

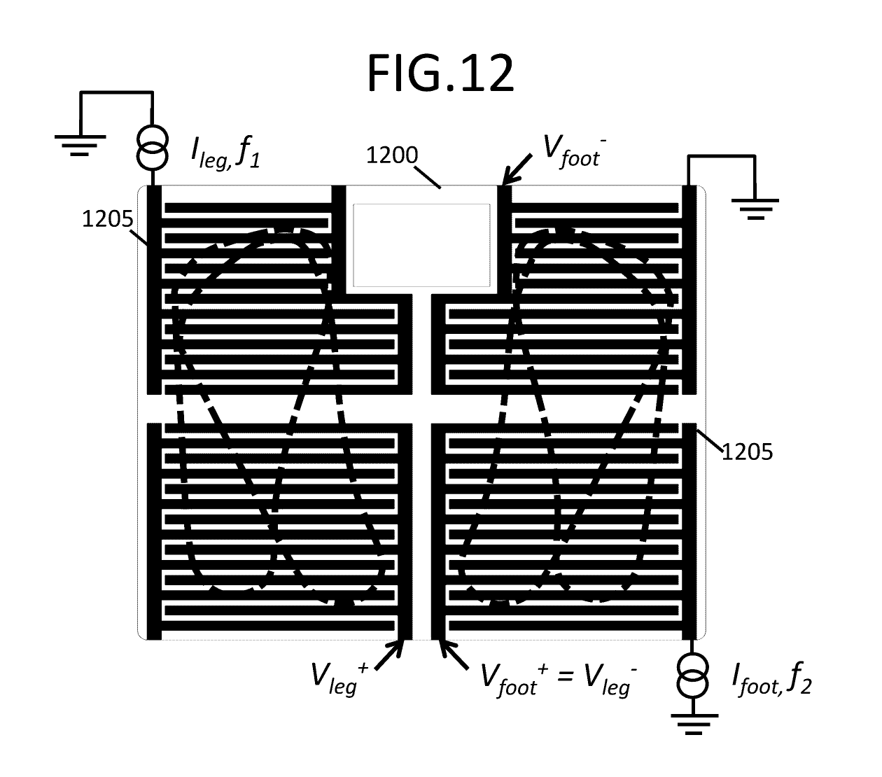

FIG. 12 shows an example of a scale 1200 with interleaved foot electrodes 1205 to inject and sense current from one foot to another foot, and within one foot, consistent with the present disclosure.

FIG. 13A-C3 shows various examples of a scale 1300 with interleaved foot electrodes 1305 to inject and sense current from one foot to another foot, and measure Foot IPG signals in both feet, consistent with various aspects of the present disclosure.

FIGS. 14A-D show an example breakdown of a scale 1400 with interleaved foot electrodes 1405 to inject and sense current from one foot to another foot, and within one foot, consistent with various aspects of the present disclosure.

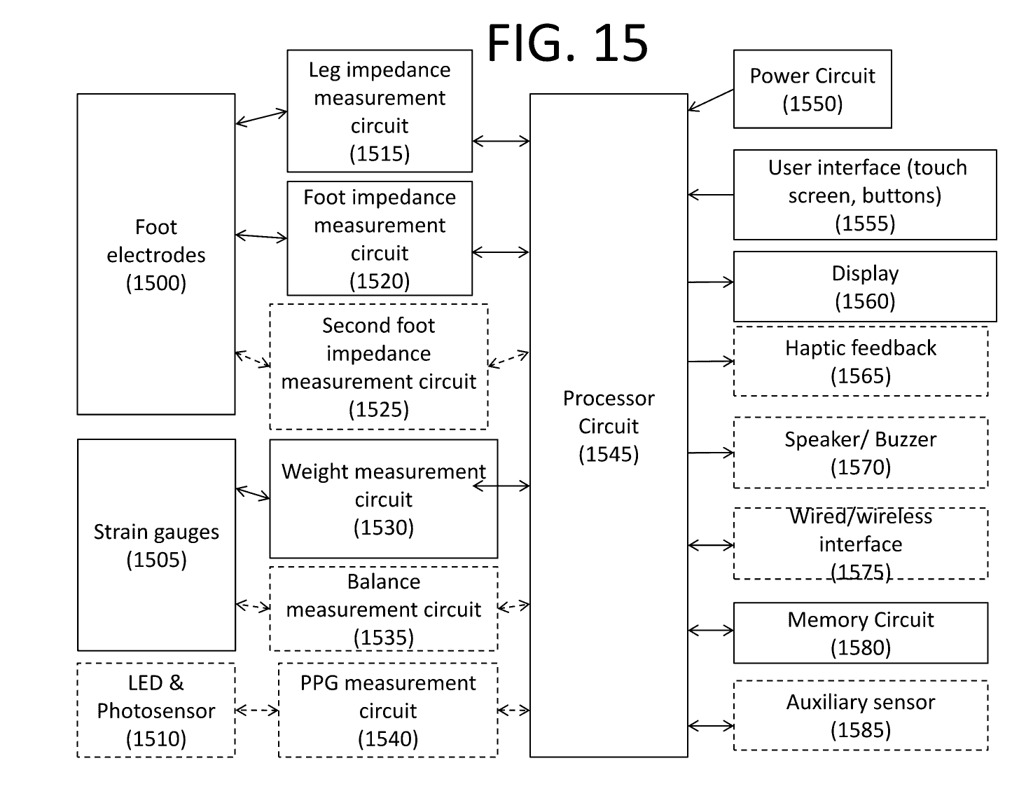

FIG. 15 shows an example block diagram of circuit-based building blocks, consistent with the present disclosure. The various circuit-based building blocks shown in FIG. 15 can be implemented in connection with the various aspects discussed herein. In the example shown, the block diagram includes foot electrodes 1500 that can collect the IPG signals. The block diagram includes strain gauges 1505, and an LED/photosensor 1510. The foot electrodes 1500 are configured with a leg impedance measurement circuit 1515, a foot impedance measurement circuit 1520, and an optional second foot impedance measurement circuit 1525. The leg impedance measurement circuit 1515, the foot impedance measurement circuit 1520, and the optional second foot impedance measurement circuit 1525 report the measurements collected to a processor circuit 1545.

The processor circuit 1545 also collects data from a weight measurement circuit 1530 and an optional balance measurement circuit 1535 that are configured with the strain gauges 1505. Further, an optional photoplethysmogram (PPG) measurement circuit 1540, which collects data from the LED/photosensor 1510, can also provide data to the processor circuit 1545.

The processor circuit 1545 is powered via a power circuit 1550. Further, the processor circuit 1545 also collects user input data from a user interface 1555 that can include a touch screen and/or buttons. The data collected/measured by the processor circuit 1545 is shown to the user via a display 1560. Additionally, the data collected/measured by the processor circuit 1545 can be stored in a memory circuit 1580. Further, the processor circuit 1545 can optionally control a haptic feedback circuit 1565, a speaker or buzzer 1570, a wired/wireless interface 1575, and an auxiliary sensor 1585.

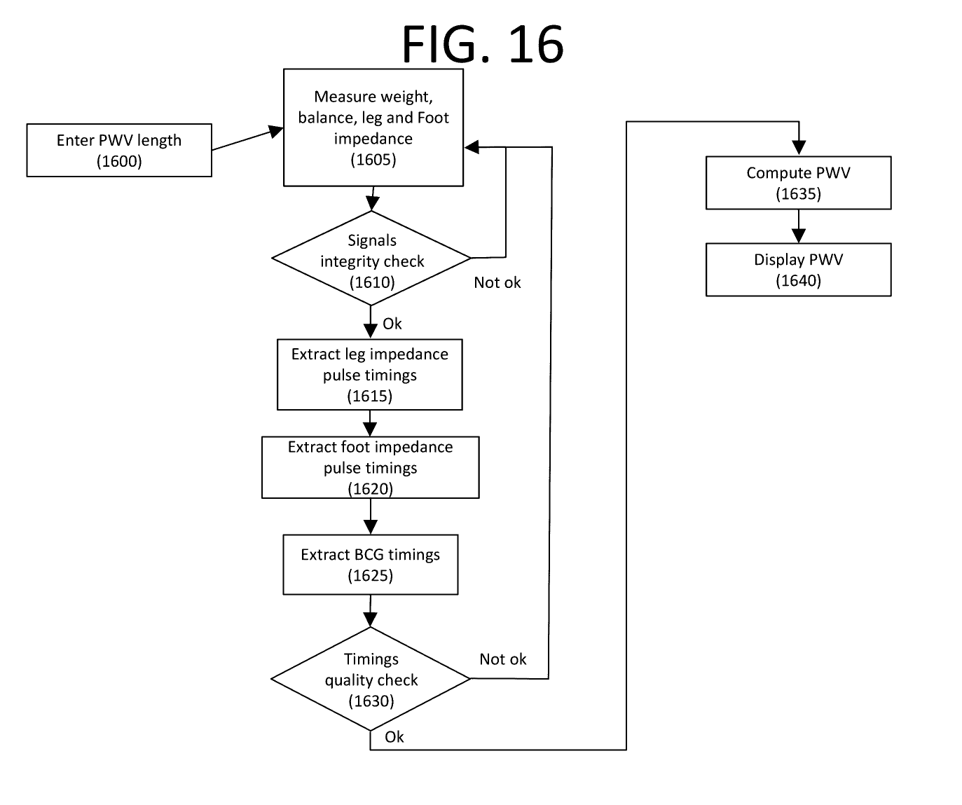

FIG. 16 shows an example flow diagram, consistent with various aspects of the present disclosure. At block 1600, a PWV length is entered. At block 1605, a user's weight, balance, leg, and foot impedance are measured. At block 1610, the integrity of signals is checked (e.g., SNR). If the signal integrity check is not met, the user's weight, balance, leg, and foot impedance are measured again (block 1605), if the signals integrity check is met, the leg impedance pulse timings are extracted (as is shown at block 1615). At block 1620, foot impedance and pulse timings are then extracted, and as is shown at block 1625, BCG timings are extracted. At block 1630, a timings quality check is performed. If the timings quality check is not validated, the user's weight, balance, leg and foot impedance are again measured (block 1605). If the timings quality check is validated, the PWV is calculated (at block 1635). At block 1640, the PWV is then displayed to the user.

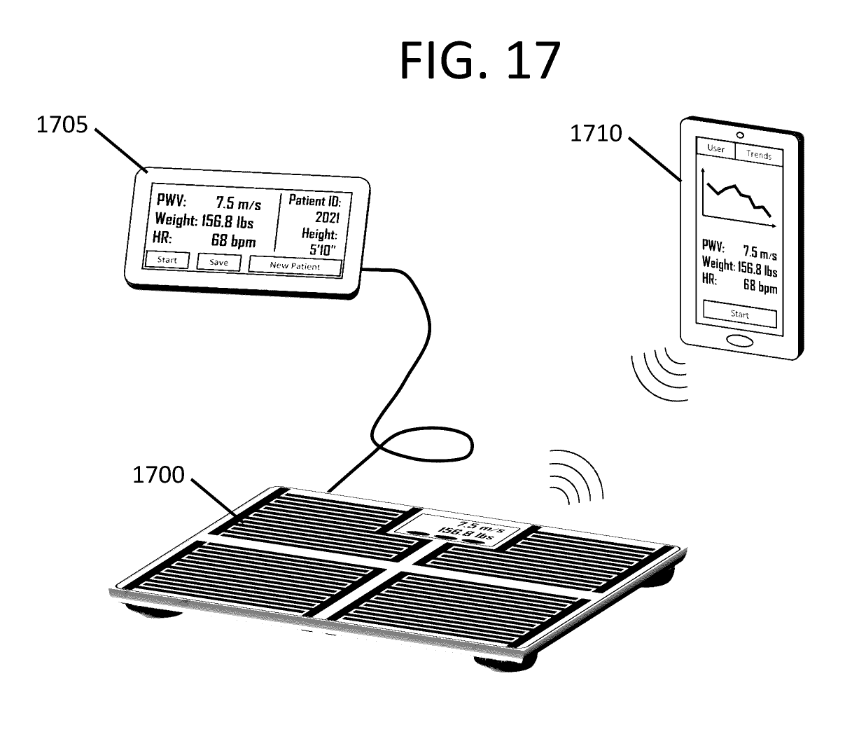

FIG. 17 shows an example scale 1700 communicatively coupled to a wireless device, consistent with various aspects of the present disclosure. As described herein, a display 1705 displays the various aspects measured by the scale 1700. The scale can also wirelessly broadcast the measurements to a wireless device 1710.

FIGS. 18A-C show example impedance as measured through different parts of the foot based on the foot position, consistent with various aspects of the present disclosure. For instance, example impedance measurement configurations may be implemented using a dynamic electrode configuration for measurement of foot impedance and related timings. Dynamic electrode configuration may be implemented using independently-configurable electrodes to optimize the impedance measurement. As shown in FIG. 18A, interleaved electrodes 1800 are connected to an impedance processor circuit 1805 to determine foot length, foot position, and/or foot impedance. As is shown in FIG. 18B, an impedance measurement is determined regardless of foot position 1810 based on measurement of the placement of the foot across the electrodes 1800. This is based in part in the electrodes 1800 that are engaged (blackened) and in contact with the foot (based on the foot position 1810), which is shown in FIG. 18C. More specifically regarding FIG. 18A, configuration can include connection/de-connection of the individual electrodes 1800 to the impedance processor circuit 1805, their configuration as current-carrying electrodes (injection or return), sense electrodes (positive or negative), or both. The configuration can either be preset based on user information, or updated at each measurement (dynamic reconfiguration) to optimize a given parameter (impedance SNR, measurement location). The system may for instance algorithmically determine which electrodes under the foot to use in order to obtain the highest SNR in the pulse impedance signal. Such optimization algorithm may include iteratively switching configurations and measuring the resulting impedance, then selecting the best-suited configuration. Alternatively, the system may first, through a sequential impedance measurement between each individual electrode 1800 and another electrode in contact with the body (such as an electrode in electrode pair 205 on the other foot), determine which electrodes are in contact with the foot. By determining the two most apart electrodes, the foot size is determined. Heel location can also be determined in this manner, as can other characteristics such as foot arch type. These parameters can then be used to determine programmatically (in an automated manner by CPU/logic circuitry) which electrodes should be selected for current injection and return (as well as sensing if a Kelvin connection issued) in order to obtain the best foot IPG.

In various embodiments involving the dynamically reconfigurable electrode array 1800/1805, an electrode array set is selected to measure the same portion/\segment of the foot, irrespective of the foot location on the array. FIG. 18B illustrates the case of several foot positions on a static array (a fixed set of electrodes are used for measurement at the heel and plantar/toe areas, with a fixed gap of an inactive electrode or insulating material between them). Depending on the position of the foot, the active electrodes are contacting the foot at different locations, thereby sensing a different volume/segment of the foot. If the IPG is used by itself (e.g., for heart measurement), such discrepancies may be non-consequential. However, if timings derived from the IPG are referred to other timings (e.g., R-wave from the ECG, or specific timing in the BCG), such as for the calculation of a PTT or PWV, the small shifts in IPG timings due to the sensing of slightly different volumes in the foot (e.g., if the foot is not always placed at the same position on the electrodes) can introduce an error in the calculation of the interval. With respect to FIG. 18B, the timing of the peak of the IPG from the foot placement on the right (sensing the toe/plantar region) is later than from the foot placement on the left, which senses more of the heel volume (the pulse reaches first the heel, then the plantar region). Factors influencing the magnitude of these discrepancies include foot shape (flat or not) and foot length.

Various embodiments address challenges relating to foot placement. FIG. 18C shows an example embodiment involving dynamic reconfiguration of the electrodes to reduce such foot placement-induced variations. As an example, by sensing the location of the heel first (as described above), it is possible to activate only a subset of electrodes under the heel, and another subset of electrodes separated by a fixed distance (1800). The other electrodes (e.g., unused electrodes) are left disconnected. The sensed volume will therefore always be the same, producing consistent timings. The electrode configuration leading to the most consistent results may also be informed by the foot impedance, foot length, the type of arch (all of which can be measured by the electrode array as shown above), but also by the user ID (foot information can be stored for each user, then looked up based on automatic user recognition or manual selection (e.g., in a look-up-table stored for each user in a memory circuit accessible by the CPU circuit in the scale)).

In certain embodiments, the apparatus measures impedance using a plurality of electrodes contacting one foot and with at least one other electrode (typically many) at a location distal from the foot. The plurality of electrodes (contacting the one foot) is arranged on the platform and in a pattern configured to inject current signals and sense signals in response thereto, for the same segment of the foot so that the timing of the pulse-based measurements does not vary simply because the user placed the one foot at a slightly different position on the platform or scale. Thus, in FIG. 18A, the foot-to-electrode locations for the heel are different locations than that shown in FIGS. 18B and 18C. As this different foot placement can occur from day to day for the user, the timing and related impedance measurements are for the same (internal) segment of the foot. By having the processor circuit inject current and sense responsive signals to first locate the foot on the electrodes (e.g., sensing where positions of the foot's heel plantar regions and/or toes), the pattern of foot-to-electrode locations permits the foot to move laterally, horizontally and both laterally and horizontally via the different electrode locations, while collecting impedance measurements relative to the same segment of the foot.

The BCG/IPG system can be used to determine the PTT of the user, by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. In certain embodiments, the BCG/IPG system is used to determine the PWV of the user, by identification of the characteristic length representing the length of the user's arteries, and by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. The system of the present disclosure and alternate embodiments may be suitable for determining the arterial stiffness (or arterial compliance) and/or cardiovascular risk of the user regardless of the position of the user's feet within the bounds of the interleaved electrodes. In certain embodiments, the weighing scale system incorporates the use of strain gage load cells and six or eight electrodes to measure a plurality of signals including: bodyweight, BCG, body mass index, fat percentage, muscle mass percentage, and body water percentage, heart rate, heart rate variability, PTT, and PWV measured simultaneously or synchronously when the user stands on the scale to provide a comprehensive analysis of the health and wellness of the user.

In other certain embodiments, the PTT and PWV are computed using timings from the Leg IPG or Foot IPG for arrival times, and using timings from a sensor located on the upper body (as opposed to the scale measuring the BCG) to detect the start of the pulse. Such sensor may include an impedance sensor for impedance cardiography, a hand-to-hand impedance sensor, a photoplethysmogram on the chest, neck, head, arms or hands, or an accelerometer on the chest (seismocardiograph) or head.

Communication of the biometric information is another aspect of the present disclosure. The biometric results from the user are stored in the memory on the scale and displayed to the user via a display on the scale, audible communication from the scale, and/or the data is communicated to a peripheral device such as a computer, smart phone, tablet computing device. The communication occurs to the peripheral device with a wired connection, or can be sent to the peripheral device through wireless communication protocols such as Bluetooth or WiFi. Computations such as signal analyses described therein may be carried out locally on the scale, in a smartphone or computer, or in a remote processor (cloud computing).

Other aspects of the present disclosure are directed toward apparatuses or methods that include the use of at least two electrodes that contact feet of a user. Further, circuitry is provided to determine a pulse arrival time at the foot based on the recording of two or more impedance signals from the set of electrodes. Additionally, a second set of circuitry is provided to extract a first pulse arrival time from a first impedance signal and use the first pulse arrival time as a timing reference to extract and process a second pulse arrival time in a second impedance signal.

Reference may also be made to the following published patent documents, U.S. Patent Publication 2010/0094147 and U.S. Patent Publication 2013/0310700, which are, together with the references cited therein, herein fully incorporated by reference for the purposes of sensors and sensing technology. The aspects discussed therein may be implemented in connection with one or more of embodiments and implementations of the present disclosure (as well as with those shown in the figures). In view of the description herein, those skilled in the art will recognize that many changes may be made thereto without departing from the spirit and scope of the present disclosure.

Various embodiments are implemented in accordance with, and fully incorporating by reference for their general teachings, the above-identified PCT Applications and U.S. Provisional Applications (including PCT Ser. No. PCT/US2016/062484 and PCT Ser. No. PCT/US2016/062505), which teachings are also incorporated by reference specifically concerning physiological scales and related measurements and communications such as exemplified by disclosure in connection with FIGS. 1a, 1b, 1c, 1d, 1e, 1f, and 2b-2e in PCT Ser. No. PCT/US2016/062484 and FIGS. 1a, 1b, 1k, 1m, and 1n in PCT. Ser. No. PCT/US2016/062505, and related disclosure in the above-identified U.S. Provisional Applications. For example, above-identified U.S. Provisional Application (Ser. No. 62/258,238), which teachings are also incorporated by reference specifically concerning obtaining derivation data, assessing a condition or treatment of the user, and drug titration features and aspects as exemplified by disclosure in connection with FIGS. 1a-1b of the underlying provisional; U.S. Provisional Application (Ser. No. 62/265,841), which teachings are also incorporated by reference specifically to controlling access to scale-obtained data that is regulated features and aspects as described in connection with FIGS. 1a-1d in the underlying provisional; and U.S. Provisional Application (Ser. No. 62/266,523), which teachings are also incorporated by reference specifically concerning to a scale having a FUI that allows the user to interact with the scale via inputs using the user's foot features and aspects as exemplified by disclosure in connection with FIGS. 1A-1B of the underlying provisional. For instance, embodiments herein and/or in the PCT and/or provisional applications may be combined in varying degrees (including wholly). Reference may also be made to the experimental teachings and underlying references provided in the PCT and/or provisional applications. Embodiments discussed in the provisional applicants are not intended, in any way, to be limiting to the overall technical disclosure, or to any part of the claimed invention unless specifically noted.

As illustrated herein, various circuit-based building blocks, aspects, features and/or modules may be implemented in various combinations to carry out one or more of the operations and activities described herein shown in the block-diagram-type figures. In such contexts, these building blocks and/or modules represent circuits that carry out one or more of these or related operations/activities. For example, in certain of the embodiments discussed above (such as the pulse circuitry modularized as shown in FIGS. 8A-B), one or more blocks/modules are discrete logic circuits or programmable logic circuits configured and arranged for implementing these operations/activities, as in the circuit blocks/modules shown. In certain embodiments, the programmable circuit is one or more computer circuits programmed to execute a set (or sets) of instructions (and/or configuration data). The instructions (and/or configuration data) can be in the form of firmware or software stored in and accessible form, a memory (circuit). As an example, first and second modules/blocks include a combination of a CPU hardware-based circuit and a set of instructions in the form of firmware, where the first module/block includes a first CPU hardware circuit with one set of instructions and the second module/block includes a second CPU hardware circuit with another set of instructions. Based upon the above discussion and illustrations, those skilled in the art will readily recognize that various modifications and changes may be made to the present disclosure without strictly following the exemplary embodiments and applications illustrated and described herein. For example, the input terminals as shown and discussed may be replaced with terminals of different arrangements, and different types and numbers of input configurations (e.g., involving different types of input circuits and related connectivity). Further, the various features and operations/actions, in accordance with various embodiments, can be combined with various different features and operations/actions and in various combinations. For example, the feature of discerning data to display on the FUI and on a GUI of external circuitry can be used in combination with configuring the text/image size based on demographic information and data to be displayed and/or color coding the data. Such modifications do not depart from the true spirit and scope of the present disclosure, including that set forth in the following claims.

What is claimed is:

1. A weighing scale comprising:
a platform configured and arranged for a user to stand on;
data-procurement circuitry, including force sensor circuitry and a plurality of electrodes integrated with the platform, and configured and arranged to engage the user with electrical signals and collect signals indicative of the user's identity and cardio-physiological measurements while the user is standing on the platform;
processing circuitry, including a CPU and a memory circuit with user-corresponding data stored in the memory circuit, configured and arranged with the data-procurement circuitry to process data obtained by the data-procurement circuitry while the user is standing on the platform and therefrom generate cardio-related physiologic data; and
a foot-controlled user interface (FUI), including circuitry, configured and arranged to provide data to the user while the user is standing on the platform and receive a foot-based user input from the user for the user to interact with the weighing scale, wherein the foot-based user input includes a movement of at least one foot of the user relative to the platform of the scale.

2. The weighing scale of claim 1, wherein the foot-based user input includes at least one movement selected from the group consisting of: the user moving their foot, the user contacting a specific portion of the platform with their foot, the user shifting their weight, and a combination thereof.

3. The weighing scale of claim 1, wherein the FUI includes a touch screen and is configured and arranged to receive the foot-based user input from the user responsive to at least one foot of the user contacting the touch screen, and wherein the foot-based user input causes the FUI to undergo a change in appearance.

4. The weighing scale of claim 1, wherein the FUI includes a user display with an icon displayed thereon and the FUI is configured to receive the foot-based user input responsive to the user moving their at least one foot to select the icon.

5. The weighing scale of claim 1, wherein the FUI includes a user display with a virtual keyboard displayed therein and is configured to receive the foot-based user input responsive to the user moving their at least one foot relative to the virtual keyboard.

6. The weighing scale of claim 1, wherein the FUI is configured to display data to confirm identification of the user and, in response to a user input, outputs the confirmation to the processing circuitry to authorize identification of the user.

7. The weighing scale of claim 1, wherein the data is provided as displayed data, and the FUI is configured to revise a size of the displayed data in response to user interaction and/or to display a size or amount of data based on user demographic information, type of data, and past user responses or inputs.

8. The weighing scale of claim 1, wherein the processing circuitry is configured and arranged to revise the FUI to personal settings of the user associated with a user profile, in response to validating identification of the user using the collected signals indicative of the user's identity.

9. The weighing scale of claim 1, wherein the platform includes a haptic, capacitive or flexible pressure-sensing upper surface, wherein processing circuitry is further configured to sense a tap of the user's foot on a upper surface of the platform and process as the foot-based user input according to X-Y grid signal processing.

10. The weighing scale of claim 1, further including one or more of accelerometers located within the platform of the weighing scale, wherein the processing circuitry is configured and arranged to sense a tap, movement, and/or pressure from at least one foot of the user as the foot-based user input.

11. The weighing scale of claim 1, further including a display configuration filter configured to discern a first portion of the user data to display to the user using the FUI of the scale based on user demographic information and an amount of data.

12. A method comprising:
transitioning a weighing scale, in response to a user standing on a platform of the scale, from a reduced power-consumption mode of operation to at least one higher power-consumption mode of operation, wherein the at least one higher power-consumption mode of operation includes activating a foot-controlled user interface (FUI), the scale including,
the platform configured and arranged for the user to stand on,
data-procurement circuitry, including force sensor circuitry and a plurality of electrodes integrated with the platform;
processing circuitry, including a CPU and a memory circuit;
the FUI including circuitry; and
an output circuit;
engaging the user with electrical signals, using the data-procurement circuitry, and collecting signals indicative of the user's identity and cardio-physiological measurements while the user is standing on the platform;
processing data, using the processing circuitry, obtained by the data-procurement circuitry while the user is standing on the platform and therefrom generating cardio-related physiologic data corresponding to the collected signals;
receiving a foot-based input, using the FUI, from the user's foot to allow the user to interact with the scale; and
providing at least the user's weight, using the FUI, to the user.

13. The method of claim 12, further including identifying the user using at least one scale-based biometric via an accelerometer within the platform of the scale.

14. The method of claim 12, further including identifying the user using at least one scale-based biometric by identifying a size of the user's foot based on the engagement of the user with the electrical signals, and the signals collected therefrom.
